SEQUENCE LISTING (25 sequences)

SEQ ID NO: 1 - DNA, 1737 bp, Ketogulonigenium sp.
atgaaatcgaattcgttgcttctggcaagcgttgctgccgttgcattctttgctgtgcccgcatttgccgatgtgacgcccgtcaccgacgagctgctagcaaacccgcccgccggcgaatggatcagctatggccgcaaccaagaaaactaccgccactcgccgctgaaccaaatcacccccgacaacgtcggccagctgcagctggtctgggcgcgcgggatgaaccccggcgtcgtgcaggtgaccccgctgatccacgacggcgtgatgtacctggcgaacccaggcgacatcattcaagcgattgacgccaaaaccggtgacctgatctgggaacaccgccgccaactgcccgagacctcgacgctcagctcgctgggggatcgcaagcgcggcatcgcgctttatggcaccaatgtctacttcgtctcgtgggacaaccacatggtcgcgctggatgctgccagcgggcaagtcgtcttcgacgtcgaccgcggccaaggcgacgagcgggtctcgaactcgtccggccccattgtggccaacggcgtgatcgtggccggttcgacctgccaatactcgcccttcggctgttttgtgtcgggccatgatgcaagcacgggcgaagaactgtggcgcaactacttcatcccgcaagcaggtgaagagggtgacgaaacctggggcaatgattacgaagcccgctggatgaccggcgtctggggccagatcacctatgaccccactactaatttggtattctacggatcgtcggccgtaggcccggcatccgaggttcagcgcggcaccccgggcggcacgctttacggcaccaacacccgctttgccgttcgtcccgacacgggcgaagtcgtctggcgtcaccaaaccctgccccgcgacaactgggaccaagagtgcacgttcgaaatgatggtcgccaatgttgacgtgcagcccgctgccgacatggacggcgtgcaagccatcaaccccaatgccgccactggcgagcgtcgcgttctgaccggcgttccgtgcaaaaccggtaccatgtggcagttcgacgctgaaacgggcgaattcctgtgggcgcgcgacaccaactaccaaaacatgatcagttcgatcgacgaaaccggtctggtcacggtgaatgaagatatcatcctaaaagatctggacaccgactaccgcatttgcccgacattcttgggtggacgcgactggccgtcggcatccttgaaccccgatagcggcatctacttcattcccctgaacaacgcctgtgcggatttggcggcagtcgatcaagagttcacggcaatggacgtctacaacaccagcgcgacttacctgcttgcgccggaaaaagaaaacatgggccgcatcgacgcgatcgacatcagcacgggcaaaaccctgtggtcggtcgaacgtctggcgtcgaactactcgccggtcctctcgacggctggcggcgtgctgttcaacggcggcagcgatcgctacttccgtgccctcagcgaggaaactggcgagaccctgtggcagacccgtctggcgactgtcgccagcggtcaagccatcagctacgaactggacggcgtgcagtatgttgccatcgcaggcggcggtaatacctacggcactaacctgaacagcaatatcggcgcgaccatcgattcgacttcgatcggcaacgccgtctacgtcttcgcccttccgcaataa

SEQ ID NO: 2 - DNA, 495 bp, Ketogulonigenium sp.
atgaacaacaaaacgattctgggcggtgttcttgctctgtcggccgttctggctggcacgacgggcgcatttgctttcagcaacatcgagcggactcccgccgctgacaccgcagctaccgaagaagccgccgcaaccgaaggtggtacgcgtaccatctacgacggcgtcttcaccgccgagcaagccgagcgtggccaaactgactggacagctagctgcgccagctgccatggcccgaccggtcgtggttcgtcgggtggcccgcgcgtgattggccccgttatcaacaacaagtacgcagacaagccgctgcaagagtacttcgactacgttgttgctaacatgccgatgggtgcgccccactcgctgagcaacgaagcctatgtcgacatcaccgccttcatcctcagctcgcacggcgcagagccgggcgatgccgagctgactgaagccgatctcggcaacatcatgatgggtcgcaagcctaactaa

SEQ ID NO: 3 - DNA, 1740 bp, Ketogulonigenium sp.
atgaagacgaagtcttttctgtttgcaggcgttgctgcgcttgcaagctacggcacaattgcgcttgctgatgtgacccccgtcaccgacgagctgctggcaaacccgcccgccggcgaatggatcagctacggccgcaaccaagaaaactatcgccactcgcccctgaaccagatcacgcccgagaacgtcggtcagctgcaactggtctgggcgcgcggcatgaacgccggcaaagtccaagtcactccgctgatccatgatggcgtgatgtacctggcgaaccccggcgacatcatccaagcgatcgacgctaaaaccggcgacctgatctgggaacaccgccgccagctgcccaacgtggcaacgctgaacagcttcggtgagccgatccgcggtatcgcgctgtacggcaccaacgtttacttcgtctcgtgggacaaccacctggttgcgctggacgcagccaccggccaagtcacgttcgacgtcgaccgcggccaaggcgaagacatggtttctaactcgtcgggcccgatcgtggctaacggcgtgatcgtggccggttcgacctgccaatactcgcccttcggctgcttcgtttcgggccatgacgcgactaccggtgaagaactgtggcgcaactacttcatccccaaagcgggtgaagaaggcgatgaaacctggggcaacgactacgaagcccgctggatgaccggcgtctggggccaaatcacgtacgaccccgtcaccaacctggtattctacggatcgtcggccgtcggcccggcttcggaaacccaacgcggcaccaccggcggcaccatgtacggcacgaacacccgtttcgccgtgcgccccgacaccggcgaaatcgtctggcgtcaccaaactctgccccgcgacaactgggaccaagagtgcacgttcgaaatgatggtcgccaatgtcgacgtccagccttcggctgacatggacggcctgaagtcgatcaaccccaacgccgccactggcgagcgtcgcgtgctgaccggcgttccgtgcaaaaccggtaccatgtggcagttcgacgctgaaacgggcgaattcctgtgggcgcgcgacaccaactaccaaaacatgatcagttcgatcgacgaaaccggtctggtcacggtgaatgaagatatcatcctaaaagatctggacaccgactaccgcatttgcccgacattcttgggtggacgcgactggccgtcggcatccttgaaccccgatagcggcatctacttcattcccctgaacaacgcctgtgcggatttggcggcagtcgatcaagagttcacggcaatggacgtctacaacaccagcgcgacttacctgcttgcgccggaaaaagaaaacatgggccgcatcgacgcgatcgacatcagcacgggcaaaaccctgtggtcggtcgaacgtctggcgtcgaactactcgccggtcctctcgacggctggcggcgtgctgttcaacggcggcagcgatcgctacttccgtgccctcagcgaggaaactggcgagaccctgtggcagacccgtctggcgactgtcgcttcgggccaagccgtgtcgtacgaactggacggcgtgcagtacatcgccatcgctggtggcggcaccacctacggcgcggtccagaaccgtccgctggccgagcctgttgactcgacctcgatcggtaacgccgtctacgttttcgctctgccccagcaataa

SEQ ID NO: 4 - DNA, 1743 bp, Ketogulonigenium sp.
atgcgacccacaacgctgcttcgcaccagcgcggccgtactattgctcggcccgatccctgcctttgcgcaggtcacccccatcaccgatgaactgctggccaacccgccagcgggcgagtggatcaactacggccggaatcaggaaaactaccgccactcgccgctggaacagattacgaccgacaacgtcggccagctgcagctggtctgggcgcgcggcatggaagcgggcgccgtgcaggtcaccccgatgatccacgacggcgtgatgtatctggccaaccccggcgacgtcatccaggccatcgacgccaaaaccggcgacctgatgtgggaacaccgccgccaactgccgcccgttgcctcgctgaacggccaaggcgaccgtaaacgcggcgtcgccctctatggcaccaacctctatttcacctcgtgggacaaccaccttgtcgcactggacatggccaccggccaagtcgtctttgatgtcgagcgcggctcgggcgatgacggcctgaccagcaacaccagcggcccgattgtcgcaaacggcgtcatcgtcgccggctcgacctgccaatactcgccctacggctgctttgtctcgggtcacgacccggccagcggcgaagaactgtggcgcaactacttcatcccgcaagcgggcgaagaaggcgacgagacctggggcaacgacttcgaatcgcgctggatgaccggcgtctggggccagctgacctatgaccccgtcaccaatctggtgcactacggctcgaccggcgtcggccccgcatccgaaacccagcgcggcaccccgggcggcacgctttacggcaccaatacccgctttgccgtgcgccccgacacgggcgaaatcgtctggcgccaccaaaccctgccccgcgacaactgggaccaggaatgcacattcgagatgatggtcgctaacgtcgacgtgcagcccgctgccgacatggacggcgttcaggccatcaaccccaacgccaccactggcgagcgtcgcgtgctgacgggcatcccctgcaaaaccggcaccatgtggcagttcgacgccgaaaccggcgaattcctgtgggcacgcgacaccaactaccagaacctgatcgcctcgatcgacgaaaccggcctggtcacggtgaacgaagacagcgtgctgacgcaactggacaccgactacgacatctgcccgaccttcctcggcggacgcgactggccgtcggcagccctgaaccccgatagcggcatctacttcataccgctgaacaacgcctgcgtcgacatcatggccgtcgatcaggaattctcggcgcttgatgtgtacaataccagcgcatcctacaagcttgcaccgggctttgaaaacatgggccgcatcgacgcgatcgacatcagcacgggcaaaaccctgtggtcggccgaacgtctggcgtcgaactactcgcccgtcctctcgacggctggcggcgtgctgttcaacggcggcaccgaccggtacttgcgcgcgctcagccaagaaaccggcgagacgctctggcagacccgtctggcgagtgtcgctaccggccaagccatcagctacgaaatcgacggcacccaatacgtcgcgatcgcggggggcggcagcacctacggcaccaaccaaaaccgtgccctcagcgaggcgatcgactcgaccacgatcggcaacgccgtttacgtttttgcgctgccgcagcagtaa

SEQ ID NO: 5 - protein, 578 aa, Ketogulonigenium sp.
MKSNSLLLASVAAVAFFAVPAFADVTPVTDELLANPPAGEWISYGRNQENYRHSPLNQITPDNVGQLQLVWARGMNPGVVQVTPLIHDGVMYLANPGDIIQAIDAKTGDLIWEHRRQLPETSTLSSLGDRKRGIALYGTNVYFVSWDNHMVALDAASGQVVFDVDRGQGDERVSNSSGPIVANGVIVAGSTCQYSPFGCFVSGHDASTGEELWRNYFIPQAGEEGDETWGNDYEARWMTGVWGQITYDPTTNLVFYGSSAVGPASEVQRGTPGGTLYGTNTRFAVRPDTGEVVWRHQTLPRDNWDQECTFEMMVANVDVQPAADMDGVQAINPNAATGERRVLTGVPCKTGTMWQFDAETGEFLWARDTNYQNMISSIDETGLVTVNEDIILKDLDTDYRICPTFLGGRDWPSASLNPDSGIYFIPLNNACADLAAVDQEFTAMDVYNTSATYLLAPEKENMGRIDAIDISTGKTLWSVERLASNYSPVLSTAGGVLFNGGSDRYFRALSEETGETLWQTRLATVASGQAISYELDGVQYVAIAGGGNTYGTNLNSNIGATIDSTSIGNAVYVFALPQ

SEQ ID NO: 6 - protein, 164 aa, Ketogulonigenium sp.
MNNKTILGGVLALSAVLAGTTGAFAFSNIERTPAADTAATEEAAATEGGTRTIYDGVFTAEQAERGQTDWTASCASCHGPTGRGSSGGPRVIGPVINNKYADKPLQEYFDYVVANMPMGAPHSLSNEAYVDITAFILSSHGAEPGDAELTEADLGNIMMGRKPN

SEQ ID NO: 7 - protein, 579 aa, Ketogulonigenium sp.
MKTKSFLFAGVAALASYGTIALADVTPVTDELLANPPAGEWISYGRNQENYRHSPLNQITPENVGQLQLVWARGMNAGKVQVTPLIHDGVMYLANPGDIIQAIDAKTGDLIWEHRRQLPNVATLNSFGEPIRGIALYGTNVYFVSWDNHLVALDAATGQVTFDVDRGQGEDMVSNSSGPIVANGVIVAGSTCQYSPFGCFVSGHDATTGEELWRNYFIPKAGEEGDETWGNDYEARWMTGVWGQITYDPVTNLVFYGSSAVGPASETQRGTTGGTMYGTNTRFAVRPDTGEIVWRHQTLPRDNWDQECTFEMMVANVDVQPSADMDGLKSINPNAATGERRVLTGVPCKTGTMWQFDAETGEFLWARDTNYQNMISSIDETGLVTVNEDIILKDLDTDYRICPTFLGGRDWPSASLNPDSGIYFIPLNNACADLAAVDQEFTAMDVYNTSATYLLAPEKENMGRIDAIDISTGKTLWSVERLASNYSPVLSTAGGVLFNGGSDRYFRALSEETGETLWQTRLATVASGQAVSYELDGVQYIAIAGGGTTYGAVQNRPLAEPVDSTSIGNAVYVFALPQQ

SEQ ID NO: 8 - protein, 580 aa, Ketogulonigenium sp.
MRPTTLLRTSAAVLLLGPIPAFAQVTPITDELLANPPAGEWINYGRNQENYRHSPLEQITTDNVGQLQLVWARGMEAGAVQVTPMIHDGVMYLANPGDVIQAIDAKTGDLMWEHRRQLPPVASLNGQGDRKRGVALYGTNLYFTSWDNHLVALDMATGQVVFDVERGSGDDGLTSNTSGPIVANGVIVAGSTCQYSPYGCFVSGHDPASGEELWRNYFIPQAGEEGDETWGNDFESRWMTGVWGQLTYDPVTNLVHYGSTGVGPASETQRGTPGGTLYGTNTRFAVRPDTGEIVWRHQTLPRDNWDQECTFEMMVANVDVQPAADMDGVQAINPNATTGERRVLTGIPCKTGTMWQFDAETGEFLWARDTNYQNLIASIDETGLVTVNEDSVLTQLDTDYDICPTFLGGRDWPSAALNPDSGIYFIPLNNACVDIMAVDQEFSALDVYNTSASYKLAPGFENMGRIDAIDISTGKTLWSAERLASNYSPVLSTAGGVLFNGGTDRYLRALSQETGETLWQTRLASVATGQAISYEIDGTQYVAIAGGGSTYGTNQNRALSEAIDSTTIGNAVYVFALPQQ

SEQ ID NO: 9 - DNA, 2519 bp, Ketogulonigenium sp.
atttgcggcataggtcgccgcgttatcgggatcttgcgcgacaaaggccttttcgatgttatcaatatagatcagcgcgttgtccaagctcatccacgcatgggggttgggtttaccctgatattccccgccagagatcgacatcggctcgatcccatccgtcagcacggcggacggaacatcgcccatgttttgcaggaattgcgcgaaccatacctcgaggttgaagccgttccacaagatcaaatcggcgccctgtgcggccaccagatcgcggggggtcggggaatagctgtggatgtcgacaccgggcttgatcagtgacacgacatccgccgcatcccccgcaacattcgacgccatgtcggccaagatggtgaatgtcgtgacaaccttcatacggccgtccgcgccttgggcagccgcctcctgcgcccaaactgccagaagtgcagcagcagccaccgccgtaccgcgcccgaatagaactaacatcactaacctctttcattaccttgcgtccgccaccatagttgcgagtcgttctcaactcaagcaaaaatgcgaacaattcgcaactacgcggaaccgccctagtcaccaactgaatgactcgcattttcgtgattttgcacttgaactcgtgcgcgaaatgtcacagcgtcagattgtcgcatctttgcgactgcgcggacggaaactctcgggaggagcatggccgtccgcgcagaaccatctggaggacagagatgaaatcgaattcgttgcttctggcaagcgttgctgccgttgcattctttgctgtgcccgcatttgccgatgtgacgcccgtcaccgacgagctgctagcaaacccgcccgccggcgaatggatcagctatggccgcaaccaagaaaactaccgccactcgccgctgaaccaaatcacccccgacaacgtcggccagctgcagctggtctgggcgcgcgggatgaaccccggcgtcgtgcaggtgaccccgctgatccacgacggcgtgatgtacctggcgaacccaggcgacatcattcaagcgattgacgccaaaaccggtgacctgatctgggaacaccgccgccaactgcccgagacctcgacgctcagctcgctgggggatcgcaagcgcggcatcgcgctttatggcaccaatgtctacttcgtctcgtgggacaaccacatggtcgcgctggatgctgccagcgggcaagtcgtcttcgacgtcgaccgcggccaaggcgacgagcgggtctcgaactcgtccggccccattgtggccaacggcgtgatcgtggccggttcgacctgccaatactcgcccttcggctgttttgtgtcgggccatgatgcaagcacgggcgaagaactgtggcgcaactacttcatcccgcaagcaggtgaagagggtgacgaaacctggggcaatgattacgaagcccgctggatgaccggcgtctggggccagatcacctatgaccccactactaatttggtattctacggatcgtcggccgtaggcccggcatccgaggttcagcgcggcaccccgggcggcacgctttacggcaccaacacccgctttgccgttcgtcccgacacgggcgaagtcgtctggcgtcaccaaaccctgccccgcgacaactgggaccaagagtgcacgttcgaaatgatggtcgccaatgttgacgtgcagcccgctgccgacatggacggcgtgcaagccatcaaccccaatgccgccactggcgagcgtcgcgttctgaccggcgttccgtgcaaaaccggtaccatgtggcagttcgacgctgaaacgggcgaattcctgtgggcgcgcgacaccaactaccaaaacatgatcagttcgatcgacgaaaccggtctggtcacggtgaatgaagatatcatcctaaaagatctggacaccgactaccgcatttgcccgacattcttgggtggacgcgactggccgtcggcatccttgaaccccgatagcggcatctacttcattcccctgaacaacgcctgtgcggatttggcggcagtcgatcaagagttcacggcaatggacgtctacaacaccagcgcgacttacctgcttgcgccggaaaaagaaaacatgggccgcatcgacgcgatcgacatcagcacgggcaaaaccctgtggtcggtcgaacgtctggcgtcgaactactcgccggtcctctcgacggctggcggcgtgctgttcaacggcggcagcgatcgctacttccgtgccctcagcgaggaaactggcgagaccctgtggcagacccgtctggcgactgtcgccagcggtcaagccatcagctacgaactggacggcgtgcagtatgttgccatcgcaggcggcggtaatacctacggcactaacctgaacagcaatatcggcgcgaccatcgattcgacttcgatcggcaacgccgtctacgtcttcgcccttccgcaataagggcgaccccgcgggaaaccgcaaccttgctgt

SEQ ID NO: 10 - DNA, 3200 bp, Ketogulonigenium sp.
taacaacaaagctgcctaggcgaaaagccccgcttcgcagctgattgcgcgaaaacttgctgaacacaccgtgatcggccgcgtgtttgccccggatgcgatcacattcagccgatcagaccgcggatttcgcgcttcgggcgggcacaatttcgcacattcgcgccatgacaggccgcgaatcaccccggaaaccgcccctgccggcgtgttgccaccagtttggcgcggcgatcacaattattctaccccgtcgcgggcgcgtgacgtgctgttacctgccaatcacacaaatgcgatgaaaaatttcttcctgcgacaaatcgcgatctttgatcatcagcatgggcaacactgcgcggcgcactgcgcacggatcgcagggttagaaccatttagctactaagttaactgcgcatacagcaaattgtcgcacgtttagggccgaatccgcaccccgccgcgcacttgacttcacacccacaaaaatgtgctgtgccgccaatcccggcgccatcgacgacgactggggggctgaaacacccgaggctgagttgcccggcaaagactgacattcctgtttcatacgtctatatagggcgtgcatgcaggtgtcgggaccttgcccggatctgcaccgcagcaaggtaaaggaagctaaaatgaacaacaaaacgattctgggcggtgttcttgctctgtcggccgttctggctggcacgacgggcgcatttgctttcagcaacatcgagcggactcccgccgctgacaccgcagctaccgaagaagccgccgcaaccgaaggtggtacgcgtaccatctacgacggcgtcttcaccgccgagcaagccgagcgtggccaaactgactggacagctagctgcgccagctgccatggcccgaccggtcgtggttcgtcgggtggcccgcgcgtgattggccccgttatcaacaacaagtacgcagacaagccgctgcaagagtacttcgactacgttgttgctaacatgccgatgggtgcgccccactcgctgagcaacgaagcctatgtcgacatcaccgccttcatcctcagctcgcacggcgcagagccgggcgatgccgagctgactgaagccgatctcggcaacatcatgatgggtcgcaagcctaactaaggccagccaccccgggaatggtcggacgctatgcgctgcattccaacccctttaccccaaaaacccaaatcaaggtcaaaccgatgaagacgaagtcttttctgtttgcaggcgttgctgcgcttgcaagctacggcacaattgcgcttgctgatgtgacccccgtcaccgacgagctgctggcaaacccgcccgccggcgaatggatcagctacggccgcaaccaagaaaactatcgccactcgcccctgaaccagatcacgcccgagaacgtcggtcagctgcaactggtctgggcgcgcggcatgaacgccggcaaagtccaagtcactccgctgatccatgatggcgtgatgtacctggcgaaccccggcgacatcatccaagcgatcgacgctaaaaccggcgacctgatctgggaacaccgccgccagctgcccaacgtggcaacgctgaacagcttcggtgagccgatccgcggtatcgcgctgtacggcaccaacgtttacttcgtctcgtgggacaaccacctggttgcgctggacgcagccaccggccaagtcacgttcgacgtcgaccgcggccaaggcgaagacatggtttctaactcgtcgggcccgatcgtggctaacggcgtgatcgtggccggttcgacctgccaatactcgcccttcggctgcttcgtttcgggccatgacgcgactaccggtgaagaactgtggcgcaactacttcatccccaaagcgggtgaagaaggcgatgaaacctggggcaacgactacgaagcccgctggatgaccggcgtctggggccaaatcacgtacgaccccgtcaccaacctggtattctacggatcgtcggccgtcggcccggcttcggaaacccaacgcggcaccaccggcggcaccatgtacggcacgaacacccgtttcgccgtgcgccccgacaccggcgaaatcgtctggcgtcaccaaactctgccccgcgacaactgggaccaagagtgcacgttcgaaatgatggtcgccaatgtcgacgtccagccttcggctgacatggacggcctgaagtcgatcaaccccaacgccgccactggcgagcgtcgcgtgctgaccggcgttccgtgcaaaaccggtaccatgtggcagttcgacgctgaaacgggcgaattcctgtgggcgcgcgacaccaactaccaaaacatgatcagttcgatcgacgaaaccggtctggtcacggtgaatgaagatatcatcctaaaagatctggacaccgactaccgcatttgcccgacattcttgggtggacgcgactggccgtcggcatccttgaaccccgatagcggcatctacttcattcccctgaacaacgcctgtgcggatttggcggcagtcgatcaagagttcacggcaatggacgtctacaacaccagcgcgacttacctgcttgcgccggaaaaagaaaacatgggccgcatcgacgcgatcgacatcagcacgggcaaaaccctgtggtcggtcgaacgtctggcgtcgaactactcgccggtcctctcgacggctggcggcgtgctgttcaacggcggcagcgatcgctacttccgtgccctcagcgaggaaactggcgagaccctgtggcagacccgtctggcgactgtcgcttcgggccaagccgtgtcgtacgaactggacggcgtgcagtacatcgccatcgctggtggcggcaccacctacggcgcggtccagaaccgtccgctggccgagcctgttgactcgacctcgatcggtaacgccgtctacgttttcgctctgccccagcaataagtctggcagcgcacatcatagcaaagggccctgcggggccctttgtcatataggccagcccccttttagggcggaagacagcctttaggcgattcaaattgcgtcaaattgacgctggtgtttacccagcaatctgaataaatctttacataacaacagaagtcccgaggatatgcagatgagacgcccgaatatgtgcggatatgtcgcatcacttgcc

SEQ ID NO: 11 - DNA, 2281 bp, Ketogulonigenium sp.
gggtgacactccatccccacgcgaatagccccgcggcggccgcgggcgtaatcctccagcacgatggcgccatgttccaactggggcaaaacgcgctgcgccaaacccagcaagtattgccccgccaaagtcaggcgcagcttgtggccgtcgcgttcccacaccttcacgccatagcggtcctcgaaccgccgcatggcgtggctgacggccgattgggtcagaaacaacttctcggccgctagggtcaaactgccggtccggtcgatctcgcgcaaaatggccagcggctggatgtcgatcatgcttcatgcacccctgtcatgttcacctatctaaagacattcacctgtcatggccacgacaatacagcaaaacctacaagtcacggaaaaatcgcacatgatcacattttctcgacctctggcccttgccatcggcgtcattatgtcgcagcttcactttgtctcagccaagggcggcacgacggtgccaccccagcgcggtgaaacatcattggaggactgaaatgcgacccacaacgctgcttcgcaccagcgcggccgtactattgctcggcccgatccctgcctttgcgcaggtcacccccatcaccgatgaactgctggccaacccgccagcgggcgagtggatcaactacggccggaatcaggaaaactaccgccactcgccgctggaacagattacgaccgacaacgtcggccagctgcagctggtctgggcgcgcggcatggaagcgggcgccgtgcaggtcaccccgatgatccacgacggcgtgatgtatctggccaaccccggcgacgtcatccaggccatcgacgccaaaaccggcgacctgatgtgggaacaccgccgccaactgccgcccgttgcctcgctgaacggccaaggcgaccgtaaacgcggcgtcgccctctatggcaccaacctctatttcacctcgtgggacaaccaccttgtcgcactggacatggccaccggccaagtcgtctttgatgtcgagcgcggctcgggcgatgacggcctgaccagcaacaccagcggcccgattgtcgcaaacggcgtcatcgtcgccggctcgacctgccaatactcgccctacggctgctttgtctcgggtcacgacccggccagcggcgaagaactgtggcgcaactacttcatcccgcaagcgggcgaagaaggcgacgagacctggggcaacgacttcgaatcgcgctggatgaccggcgtctggggccagctgacctatgaccccgtcaccaatctggtgcactacggctcgaccggcgtcggccccgcatccgaaacccagcgcggcaccccgggcggcacgctttacggcaccaatacccgctttgccgtgcgccccgacacgggcgaaatcgtctggcgccaccaaaccctgccccgcgacaactgggaccaggaatgcacattcgagatgatggtcgctaacgtcgacgtgcagcccgctgccgacatggacggcgttcaggccatcaaccccaacgccaccactggcgagcgtcgcgtgctgacgggcatcccctgcaaaaccggcaccatgtggcagttcgacgccgaaaccggcgaattcctgtgggcacgcgacaccaactaccagaacctgatcgcctcgatcgacgaaaccggcctggtcacggtgaacgaagacagcgtgctgacgcaactggacaccgactacgacatctgcccgaccttcctcggcggacgcgactggccgtcggcagccctgaaccccgatagcggcatctacttcataccgctgaacaacgcctgcgtcgacatcatggccgtcgatcaggaattctcggcgcttgatgtgtacaataccagcgcatcctacaagcttgcaccgggctttgaaaacatgggccgcatcgacgcgatcgacatcagcacgggcaaaaccctgtggtcggccgaacgtctggcgtcgaactactcgcccgtcctctcgacggctggcggcgtgctgttcaacggcggcaccgaccggtacttgcgcgcgctcagccaagaaaccggcgagacgctctggcagacccgtctggcgagtgtcgctaccggccaagccatcagctacgaaatcgacggcacccaatacgtcgcgatcgcggggggcggcagcacctacggcaccaaccaaaaccgtgccctcagcgaggcgatcgactcgaccacgatcggcaacgccgtttacgtttttgcgctgccgcagcagtaaaaaccgacc

SEQ ID NO: 12 - protein, 27 aa, Ketogulonigenium robustum ADM X6L
  UNSURE (24)..(24): X can represent any amino acid
DVTPVTDELLANPPAGEWISYGGXNNX

SEQ ID NO: 13 - protein, 9 aa, Ketogulonigenium sp. ADM 291-19
QVTPVTDEL

SEQ ID NO: 14 - protein, 24 aa, Ketogulonigenium robustum ADM X6L
  UNSURE (10)..(10): X may represent any amino acid
ADTAATEEAXATEGGTRTIYDGVF

SEQ ID NO: 15 - DNA, 24 bp, Artificial Sequence
  Oligonucleotide primer
ggatccatacctcgaggttgaagc

SEQ ID NO: 16 - DNA, 22 bp, Artificial Sequence
  Oligonucleotide primer
aagcttgcggtttcccgcgggg

SEQ ID NO: 17 - DNA, 25 bp, Artificial Sequence
  Oligonucleotide primer
ggatcctaggcgaaaagccccgctt

SEQ ID NO: 18 - DNA, 24 bp, Artificial Sequence
  Oligonucleotide primer
catatgaagacgaagtcttttctg

SEQ ID NO: 19 - DNA, 23 bp, Artificial Sequence
  Oligonucleotide primer
aagcttattgctggggcagagcg

SEQ ID NO: 20 - DNA, 22 bp, Artificial Sequence
  Oligonucleotide primer
ggatccccacgcgaatagcccc

SEQ ID NO: 21 - DNA, 25 bp, Artificial Sequence
  Oligonucleotide primer
ctcgagtttttactgctgcggcagc

SEQ ID NO: 22 - DNA, 23 bp, Artificial Sequence
  Oligonucleotide primer
ccatggcgggagtccgctcgatg

SEQ ID NO: 23 - protein, 29 aa, Artificial Sequence
  Signature sequence
XWXXXGXXXXXXXXSXXXXXXXXNXXXLX

SEQ ID NO: 24 - protein, 22 aa, Artificial Sequence
  Signature sequence
WXXXXYDXXXXXXXXXXGXXXP

SEQ ID NO: 25 - protein, 5 aa, Artificial Sequence
  Signature sequence
CXXCH

(19) United States
(12) Patent Application Publication
(10) Pub. No.: US 2003/0228672 A1
(43) Pub. Date: Dec. 11, 2003

(54) Sorbitol dehydrogenases of ketogulonigenium species, genes and methods of use thereof

(75) Inventors: Eui-Sung Choi, Taejon (KR); John D'Elia, Decatur, IL (US); Hye-Sun Kim, Taejon (KR); Mi-Soo Kim, Taejon (KR); Jung Kee Lee, Taejon (KR); Jae-Gu Pan, Taejon (KR); Steven F. Stoddard, Decatur, IL (US); Do-Young Yum, Taejon (KR)

Correspondence Address: STERNE, KESSLER, GOLDSTEIN & FOX PLLC, 1100 NEW YORK AVENUE, N.W., WASHINGTON, DC, 20005, US

(73) Assignee: Archer-Daniels-Midland Company

(21) Appl. No.: 10/162,713

(22) Filed: Jun. 6, 2002

(51) Int. Cl. C12P 21/02; C07H 21/04; C12P 7/40; C12N 9/04; C12N 9/02; C12N 1/21; C12N 15/74

(52) U.S. Cl. 435/189; 435/69.1; 435/136; 435/252.3; 435/320.1; 536/23.2

(57) ABSTRACT

The invention relates to the fields of molecular biology, bacteriology and industrial fermentation. More specifically, the invention relates to the identification and isolation of nucleic acid sequences and proteins of sorbitol dehydrogenases and cytochrome c of the strains, Ketogulonigenium spp. The invention further relates to the fermentative production of L-sorbose from D-sorbitol and the subsequent production of 2-keto-L-gulonic acid.

### BACKGROUND OF THE INVENTION

1. Field of the Invention

The invention relates to the fields of molecular biology, bacteriology and industrial fermentation. More specifically, the invention relates to the identification and isolation of nucleic acid sequences and proteins of sorbitol dehydrogenases and cytochrome c of the strains, Ketogulonigenium spp. The invention further relates to the fermentative production of L-sorbose from D-sorbitol and the subsequent production of 2-keto-L-gulonic acid.

The process of the manufacturing of 2-keto-L-gulonic acid (2KLG), a precursor of vitamin C, is comprised of the process of production of L-sorbose from D-sorbitol by sorbose fermentation and the process of 2KLG production from L-sorbose. The process of manufacturing L-sorbose from D-sorbitol is typically performed by fermentation with acetic acid bacterium such as Gluconobacter suboxydans and Acetobacter xylinum. At room temperature, 96-99% of conversion is made in less than 24 hours (Liebster, J. et al., Chem. List., 50:395 (1056)). L-sorbose produced by the sorbose fermentation is a substrate in the production of 2KLG. This type of pathway of 2KLG production from D-sorbitol through consecutive oxidations of D-sorbitol into L-sorbose, L-sorbose into L-sorbosone and L-sorbosone into 2KLG is called the sorbosone pathway. A variety of processes for the production of 2KLG are known. For example, the fermentative production of 2KLG via oxidation of L-sorbose to 2KLG via a sorbosone intermediate is described for processes utilizing a wide range of bacteria: Gluconobacter suboxydans (U.S. Pat. Nos. 4,935,359; 4,960,695; 5,312,741; and 5,541,108); Pseudogluconobacter saccharoketogenes (U.S. Pat. No. 4,877,735; European Pat. No. 221 707); Pseudomonas sorbosoxidans (U.S. Pat. Nos. 4,933,289 and 4,892,823); mixtures of microorganisms from these and other genera, such as Acetobacter, Bacillus, Serratia, Mycobacterium, and Streptomyces (U.S. Pat. Nos. 3,912,592; 3,907,639; and 3,234,105); and novel bacterial strains (U.S. Pat. No. 5,834,231).

These processes, however, suffer from certain disadvantages that limit their usefulness for commercial production of 2-KLG. For example, the processes referenced above that employ G. oxydans also require the presence of an additional “helper” microbial strain, such as Bacillus megaterium, or commercially unattractive quantities of yeast or growth components derived from yeast in order to produce sufficiently high levels of 2-KLG for commercial use. Similarly, the processes that employ Pseudogluconobacter can require medium supplemented with expensive and unusual rare earth salts or the presence of a helper strain, such as B. megaterium, and/or the presence of yeast in order to achieve commercially suitable 2-KLG concentrations and efficient use of sorbose substrate. Other processes that employ Pseudomonas sorbosoxidans also include commercially unattractive qualities of yeast or yeast extract in the medium.

A number of enzymes involved in the fermentative oxidation of D-sorbitol, L-sorbose, and L-sorbosone are identified in the literature. U.S. Pat. Nos. 5,888,786; 5,861,292; 5,834,263 and 5,753,481 disclose nucleic acid molecules encoding and/or isolated proteins for L-sorbose dehydrogenase and L-sorbosone dehydrogenase; and U.S. Pat. No. 5,747,301 discloses an enzyme with specificity for D-sorbitol dehydrogenase.

In an effort to improve the productivity of commercial fermentation in the production of 2KLG, the inventors have identified sorbitol dehydrogenases in several strains of novel isolates including Ketogulonigenium robustum ADM X6L and Ketogulonigenium sp. ADM 291-19 (U.S. Pat. Nos. 5,834,231 and 5,989,891) that can efficiently produce 2KLG. These newly identified sorbitol dehydrogenases contain dehydrogenase activity toward all the substrates in the so-called sorbosone pathway. The inventors also cloned three genes encoding the dehydrogenases and one gene encoding the cytochrome c, an electron acceptor of the dehydrogenases.

### SUMMARY OF THE INVENTION

These and other objects are accomplished by the methods of the present invention, which, are directed to a processes for producing 2-KLG from L-sorbose, which comprises the steps of culturing in a medium a microorganism of strain NRRL B-21627 (ADM X6L) or a mutant or variant thereof, either alone or in mixed culture with one or more helper strains, and then recovering the accumulated 2-KLG.

This invention pertains to novel sorbitol dehydrogenases (SDH) of Ketogulonigenium spp. The SDH enzymes of this invention utilize each of D-sorbitol, L-sorbose and L-sorbosone as substrates. Thus the SDH enzymes of the invention catalyze the dehydrogenation reactions of all three sugar intermediates involved in the production of 2-keto-L-gulonic acid (2KLG) from D-sorbitol, i.e., L-sorbose and L-sorbosone (tested with glyoxal as an alternative substrate) as well as D-sorbitol. The isolated sorbitol dehydrogenase enzymes have the apparent molecular weight of 62-64 kDa and showed a very broad substrate spectrum. The present invention also pertains to a cytochrome c, a natural electron acceptor of the dehydrogenase.

The present invention provides nucleic acid molecules that encode each of three SDH enzymes, SDH1, SDH2 and SDH3, of Ketogulonigenium spp. described herein including Ketogulonigenium robustum ADM X6L and Ketogulonigenium sp. ADM 291-19 (U.S. Pat. Nos. 5,834,231 and 5,989,891). These two strains can efficiently produce 2KLG from D-sorbitol and L-sorbose. In a first embodiment, the invention provides an isolated nucleic acid encoding the polypeptide molecule identified by SEQ ID NO:5. In a second embodiment, the invention provides an isolated nucleic acid encoding the polypeptide molecule identified by SEQ ID NO:6. In a third embodiment, the invention provides an isolated nucleic acid encoding the polypeptide molecule identified by SEQ ID NO:7. In a fourth embodiment, the invention provides an isolated nucleic acid encoding the polypeptide molecule identified by SEQ ID NO:8. In a first specific embodiment, the invention provides an isolated nucleic acid molecule encoding SDH1, such nucleic acid molecule being identified by SEQ ID NO:1. In a second specific embodiment, the invention provides an isolated nucleic acid molecule encoding cytochrome c and SDH2, such nucleic acid molecule being identified by SEQ ID NOS:2 and 3, respectively. In a third specific embodiment, the invention provides an isolated nucleic acid molecule encoding SDH3, such nucleic acid molecule being identified by SEQ ID NO:4. Other related embodiments are drawn to vectors, processes for producing the same and host cells carrying said vectors.

The invention also provides isolated nucleic acid molecules encoding the three SDH enzymes of the invention. In one specific embodiment, the invention provides a cloned nucleic acid molecule encoding the SDH1. The structural gene coding for the SDH1 is 1,737 bp in size and found in a 2.9 kb HindIII/StuI DNA fragment. In another specific embodiment, the invention provides a cloned nucleic acid molecule encoding the cytochrome c and the SDH2. The structural genes coding for the cytochrome c and the SDH2 are 495 bp and 1,740 bp, respectively, in size and are clustered in the cloned nucleic acid molecule which is 5 kb BamHI/PstI DNA fragment that defines the operon. In another specific embodiment, the invention provides a cloned nucleic acid molecule encoding the SDH3. The structural gene coding for the SDH3 is 1,743 bp in size and found in a 3 kb NotI DNA fragment. Other related embodiments, are drawn to vectors, processes to make the same and host cells containing said vectors.

The invention is also drawn to purified or isolated polypeptides for the three SDH enzymes described herein, having an amino acid sequences encoded by the polynucleotides described herein.

The invention provides a method for the production of L-sorbose comprising: (a) transforming a host cell with at least one isolated nucleotide sequence selected from the group consisting of: (i) a polynucleotide encoding the amino acid sequence of SEQ ID NO:5; (ii) a polynucleotide encoding the amino acid sequence of SEQ ID NO:6; (iii) a polynucleotide encoding the amino acid sequence of SEQ ID NO:7; and (iv) a polynucleotide encoding the amino acid sequence of SEQ ID NO: 8; (b) selecting and propagating said transformed host cell; and (c) recovering L-sorbose.

The invention also provides a method for the production of L-sorbose comprising: (a) transforming a host cell with at least one isolated nucleotide sequence selected from the group consisting of: (i) a polynucleotide comprising the polynucleotide sequence of SEQ ID NO:1; (ii) a polynucleotide comprising the polynucleotide sequence of SEQ ID NO:2; (iii) a polynucleotide comprising the polynucleotide sequence of SEQ ID NO:3; and (iv) a polynucleotide comprising the polynucleotide sequence of SEQ ID NO:4; (b) selecting and propagating said transformed host cell; and (c) recovering L-sorbose.

Another aspect of the invention is drawn to a method for the production of 2KLG comprising: (a) transforming a host cell with at least one isolated nucleotide sequence selected from the group consisting of: (i) a polynucleotide encoding the amino acid sequence of SEQ ID NO:5; (ii) a polynucleotide encoding the amino acid sequence of SEQ ID NO:6; (iii) a polynucleotide encoding the amino acid sequence of SEQ ID NO:7; and (iv) a polynucleotide encoding the amino acid sequence of SEQ ID NO: 8; (b) selecting and propagating said transformed host cell; and (c) recovering 2KLG

Another aspect of the invention is drawn to a method for the production of 2KLG comprising: (a) transforming a host cell with at least one isolated nucleotide sequence selected from the group consisting of a polynucleotide comprising the polynucleotide sequence of SEQ ID NO:1; a polynucleotide comprising the polynucleotide sequence of SEQ ID NO:2; a polynucleotide comprising the polynucleotide sequence of SEQ ID NO:3; and a polynucleotide comprising the polynucleotide sequence of SEQ ID NO:4; (b) selecting and propagating said transformed host cell; and (c) recovering 2KLG.

### BRIEF DESCRIPTION OF THE FIGURES

FIGS. 1A-1I show a restriction endonuclease map of SEQ ID NO:1.

FIGS. 2A-2D show a restriction endonuclease map of SEQ ID NO:2.

FIGS. 3A-3I show a restriction endonuclease map of SEQ ID NO:3.

FIGS. 4A-4J show a restriction endonuclease map of SEQ ID NO:4.

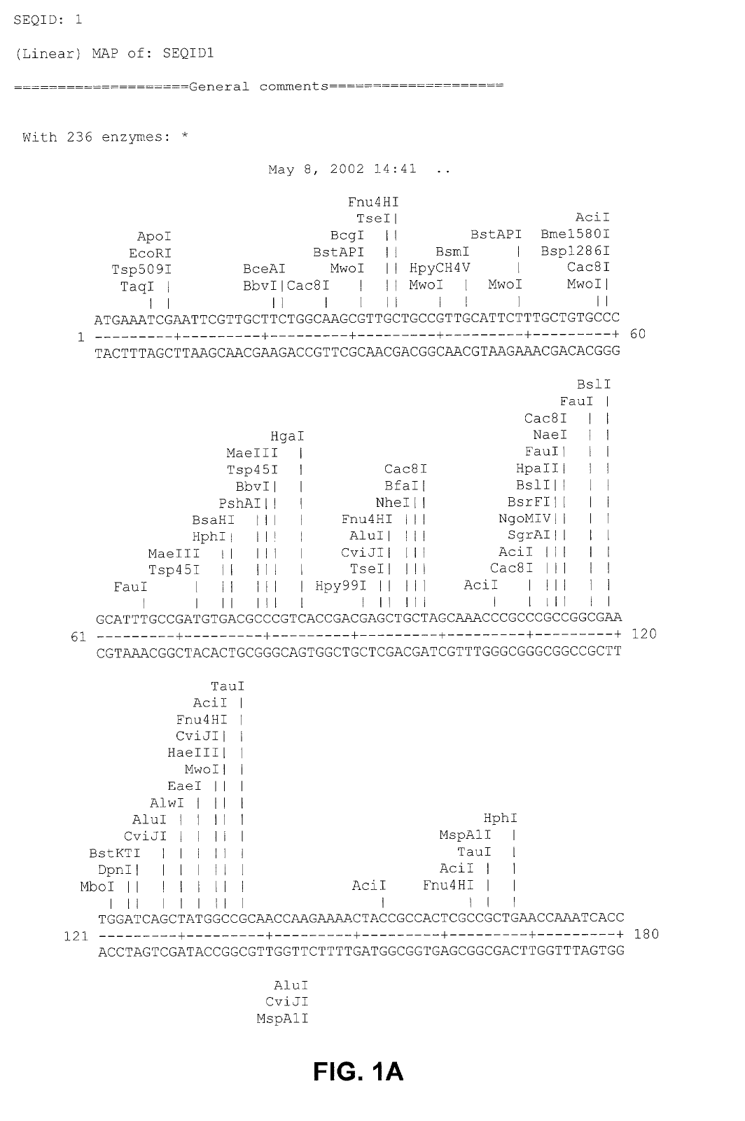

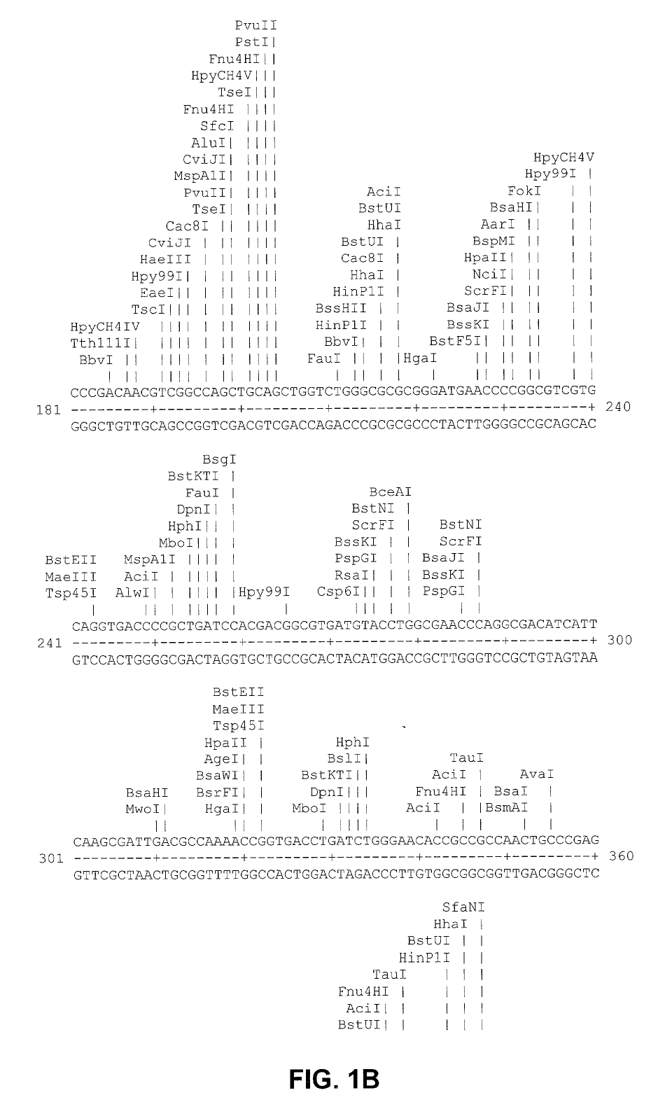

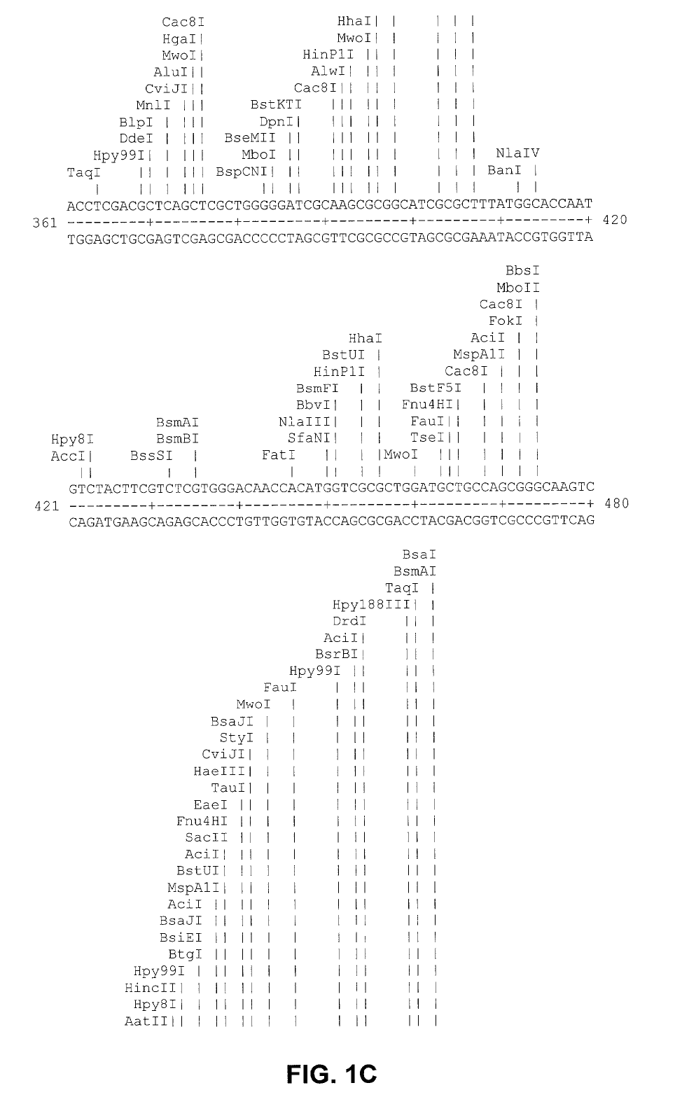

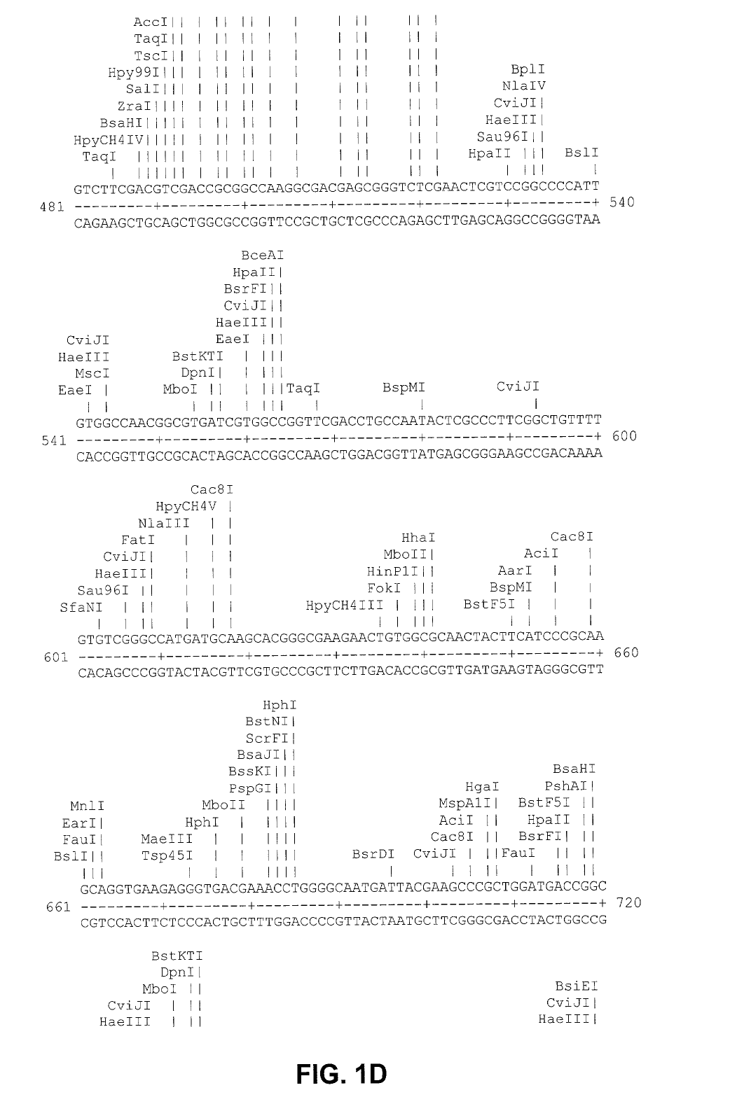

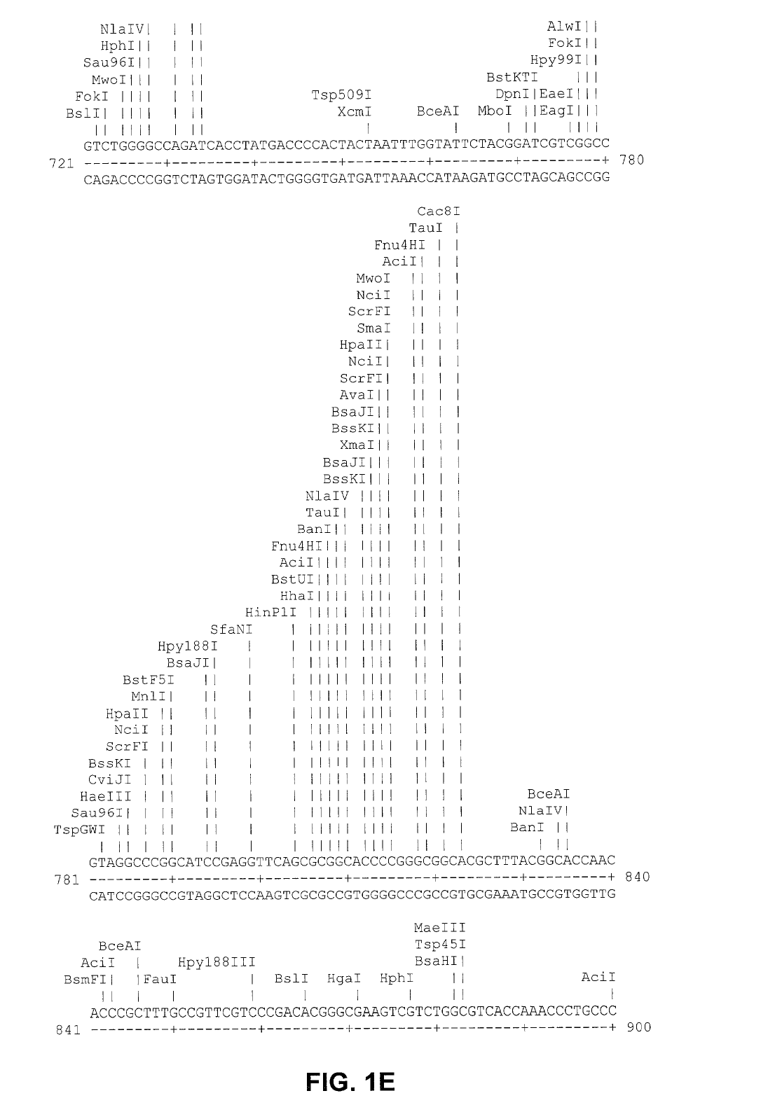

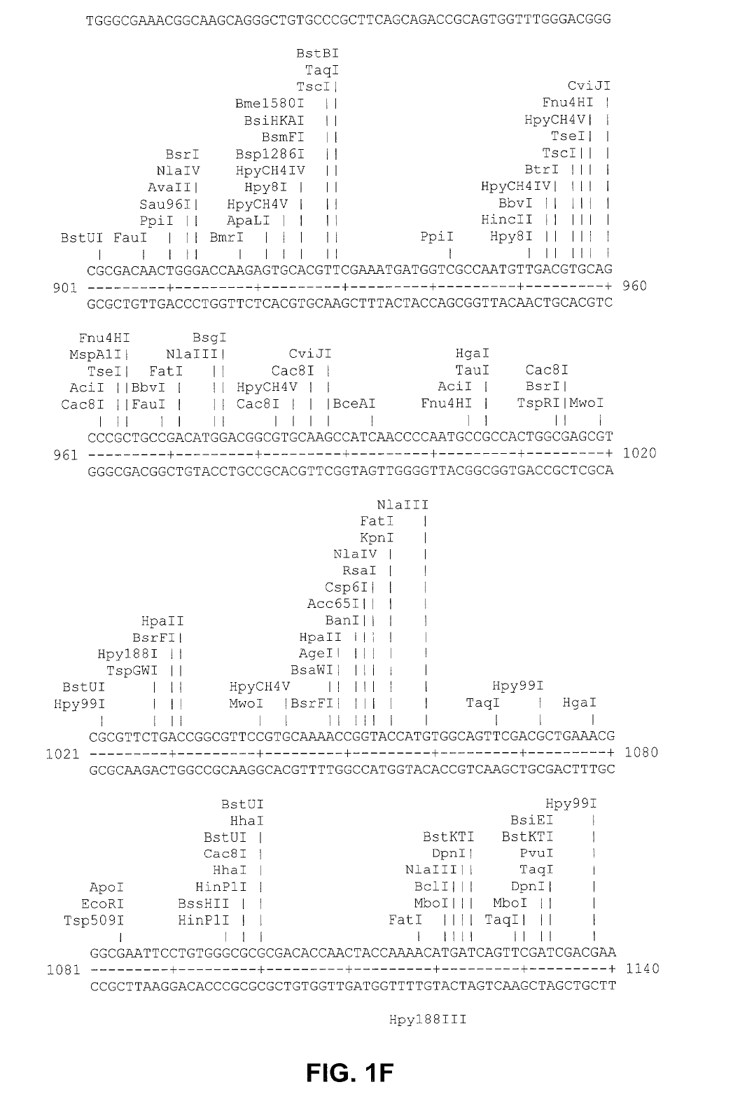

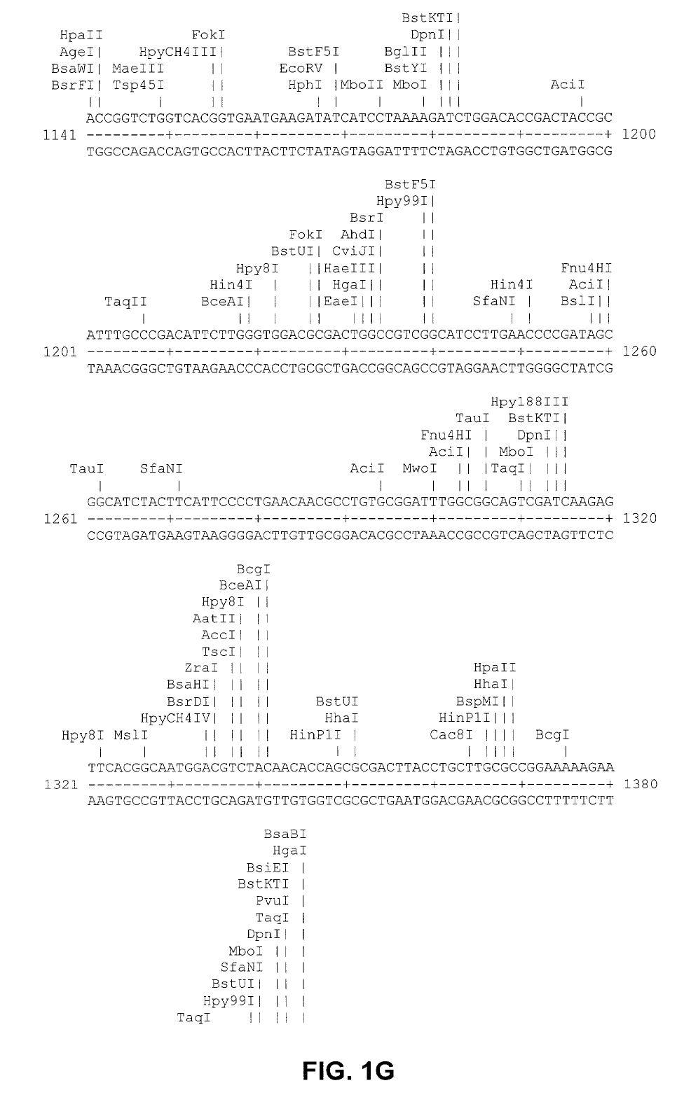

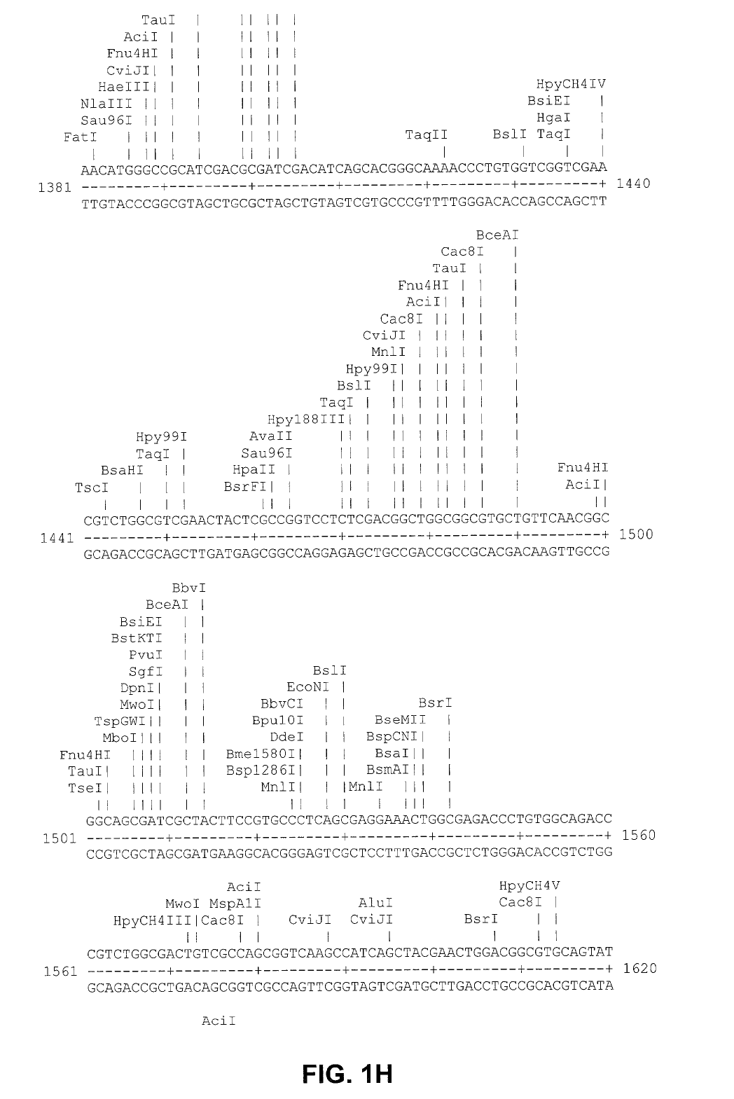

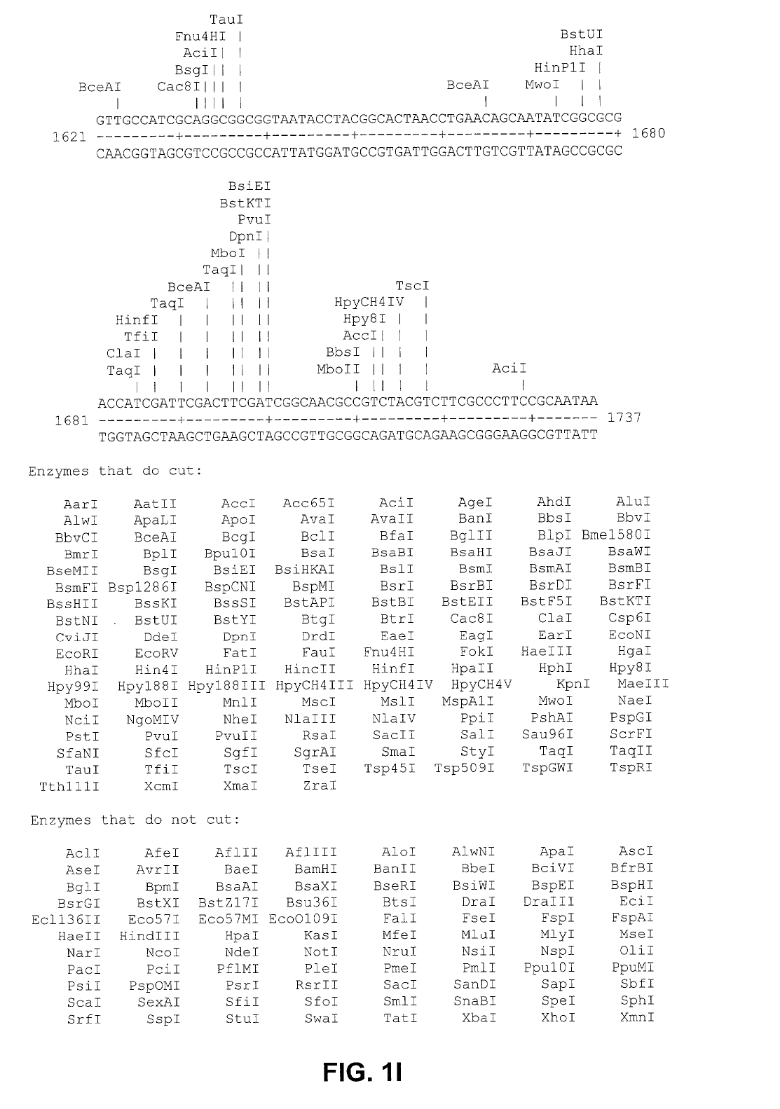

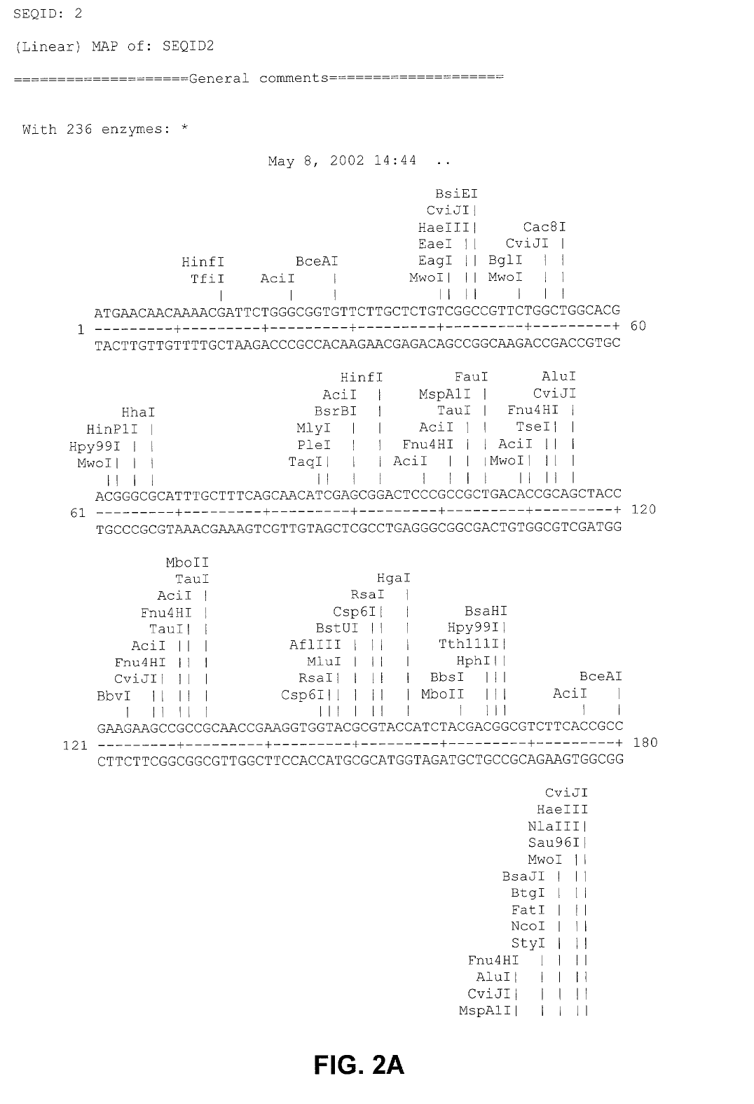

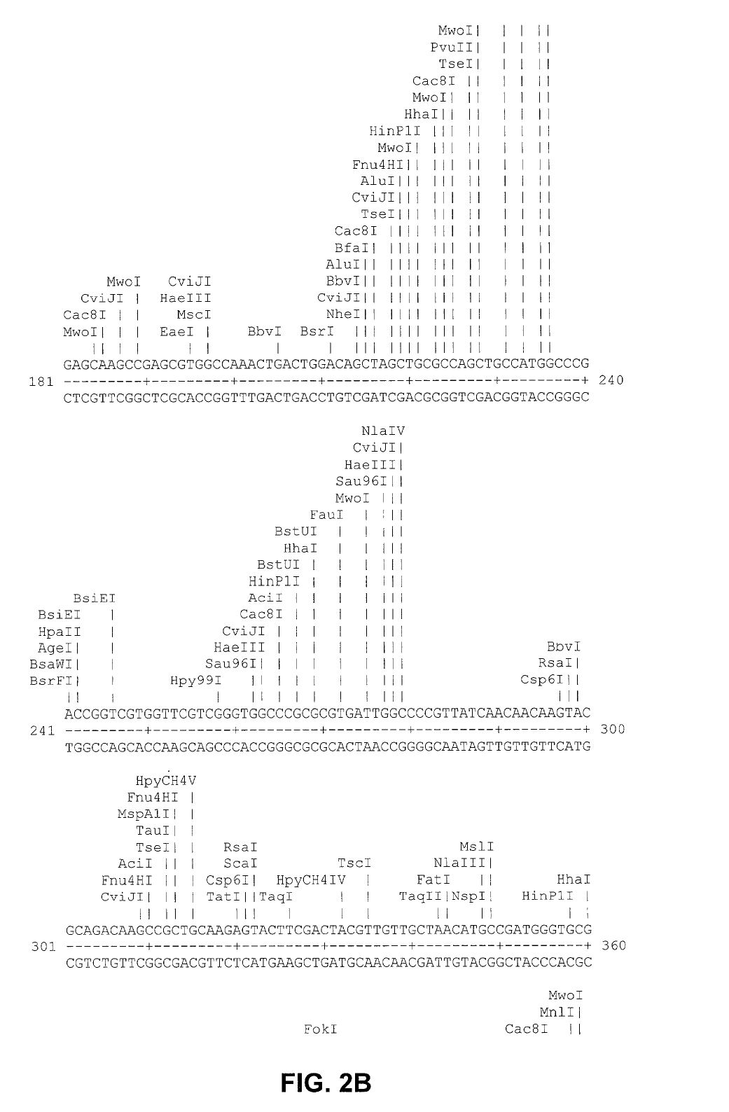

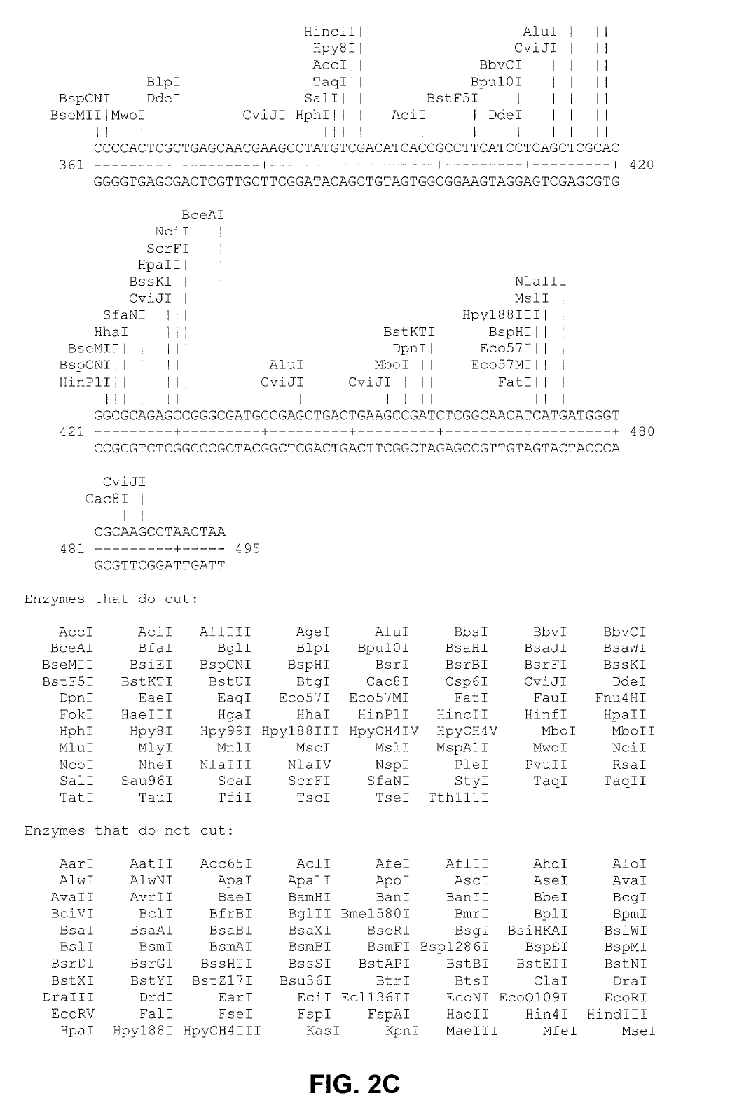

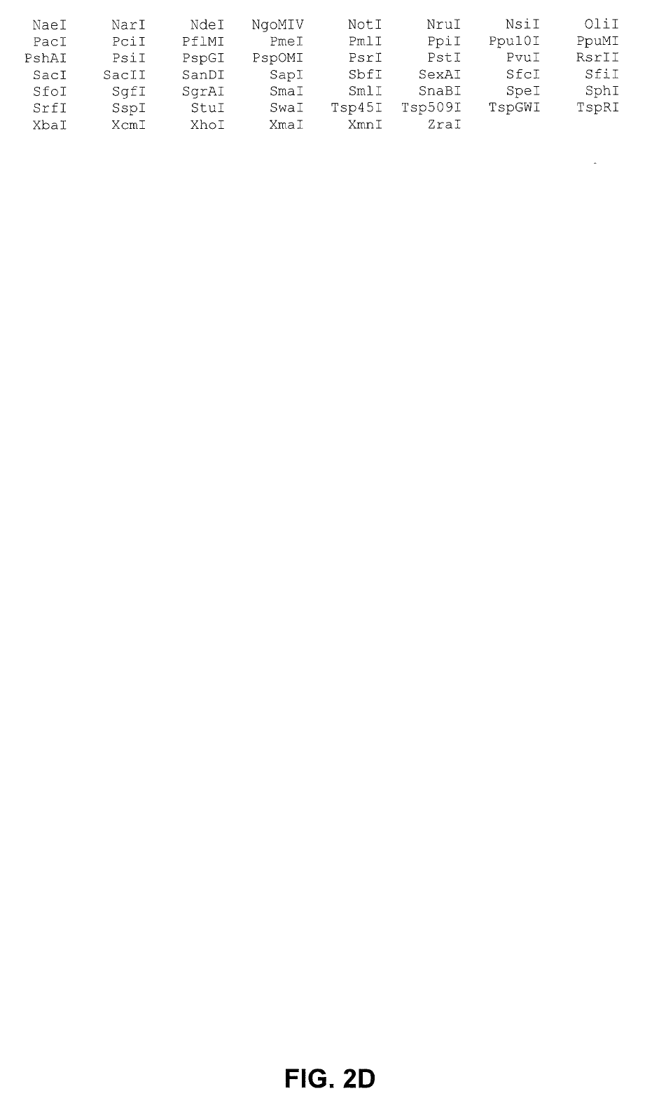

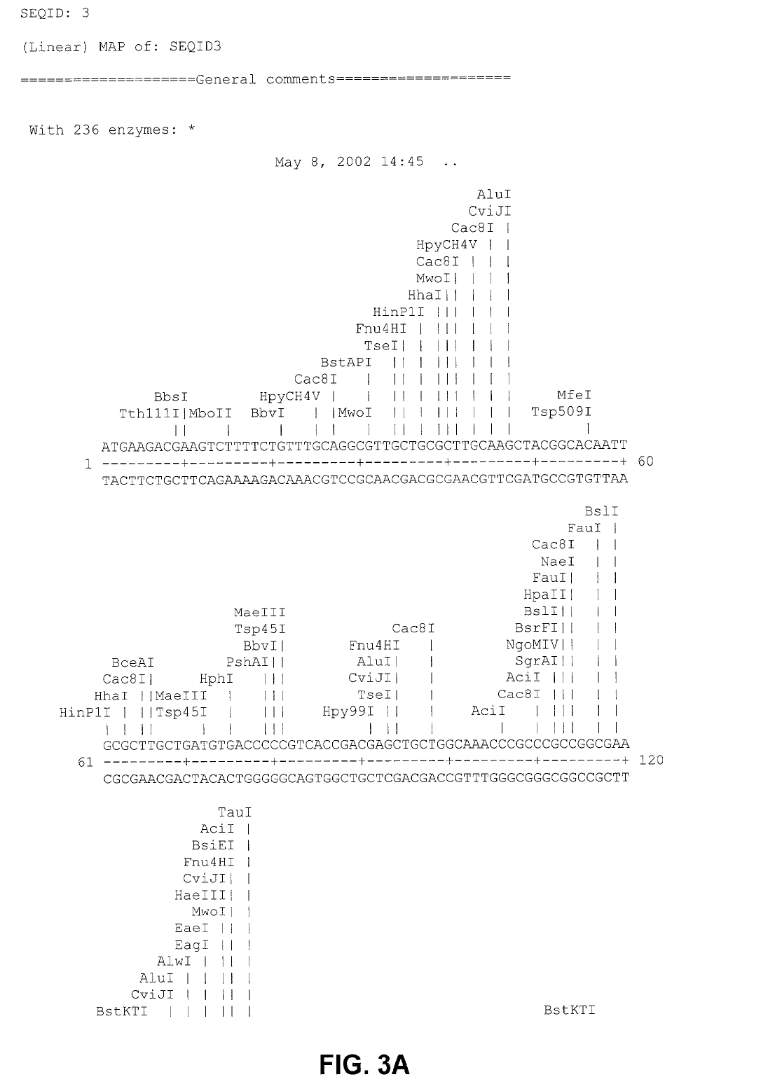

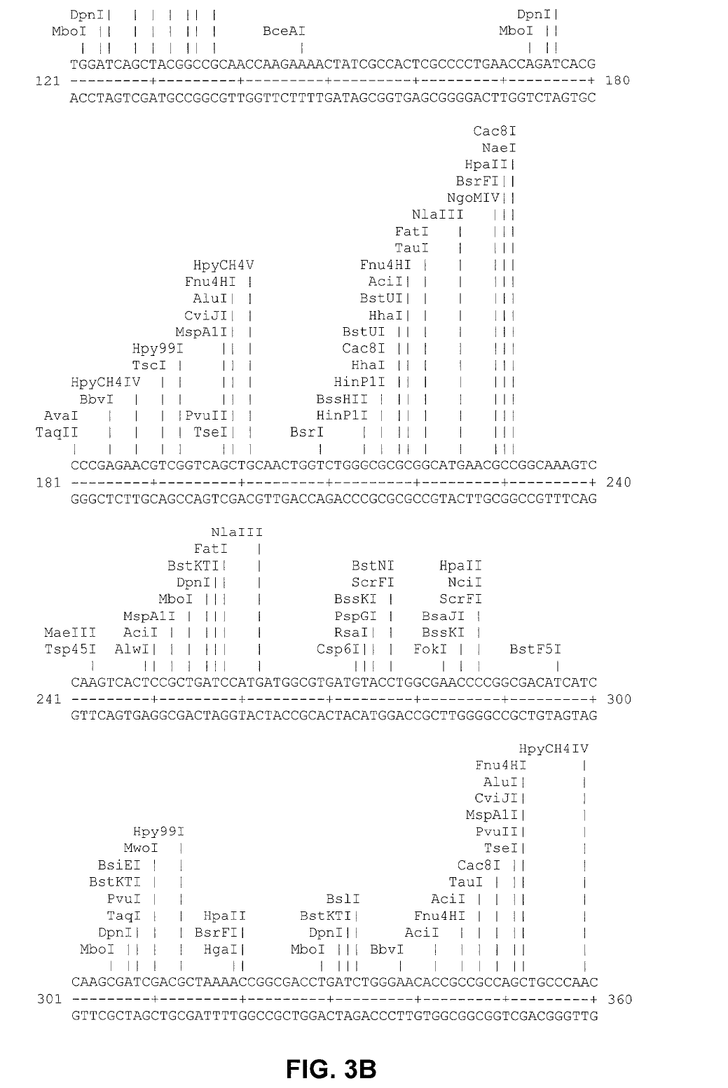

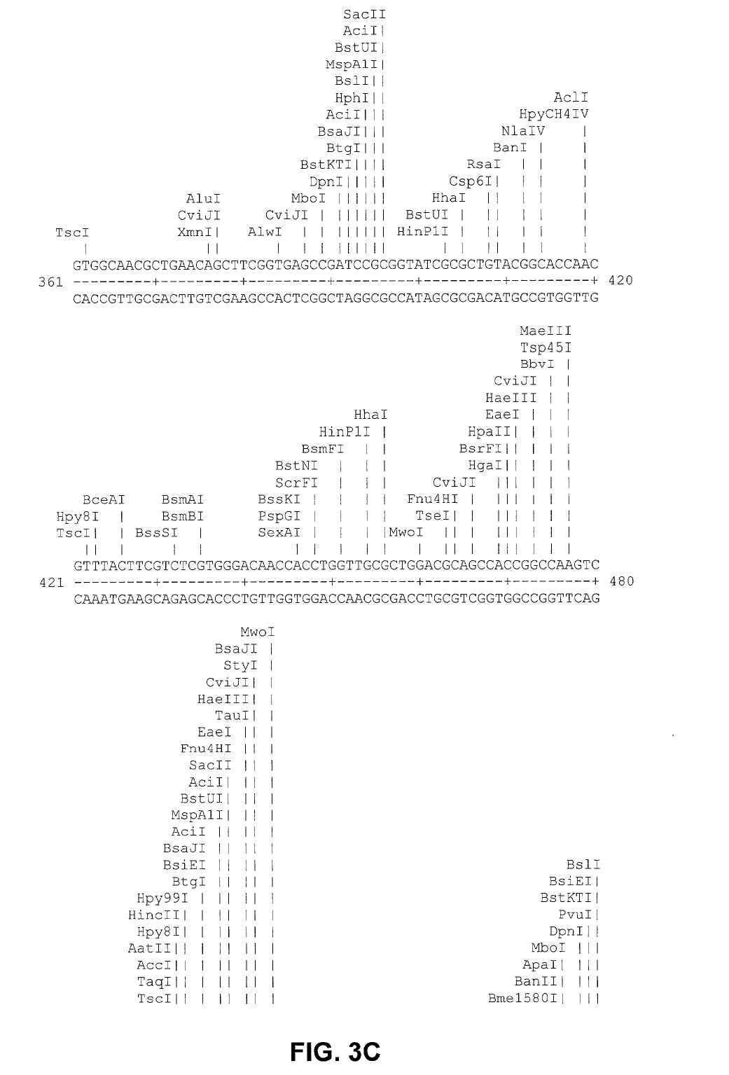

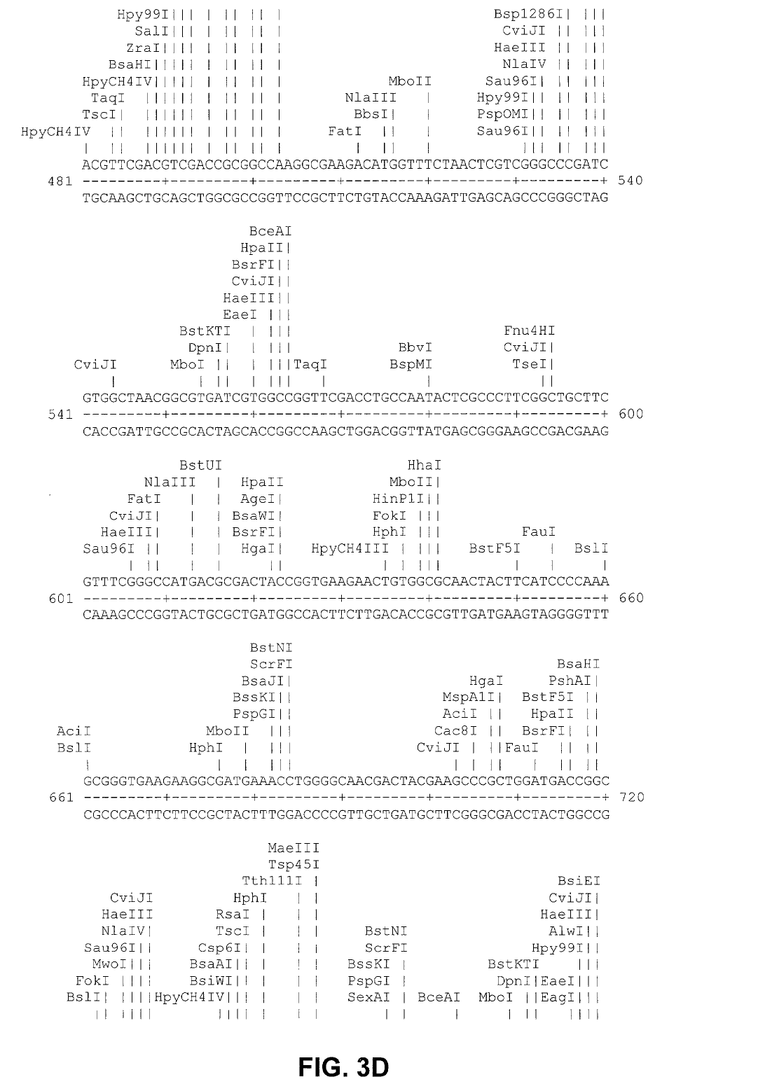

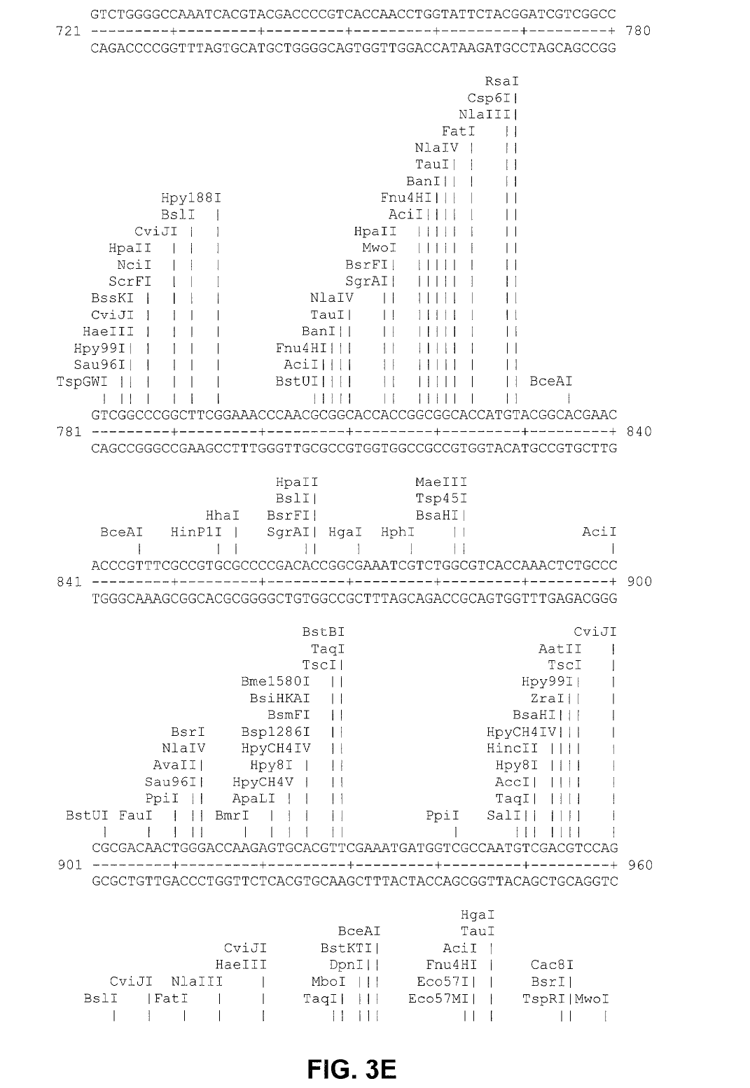

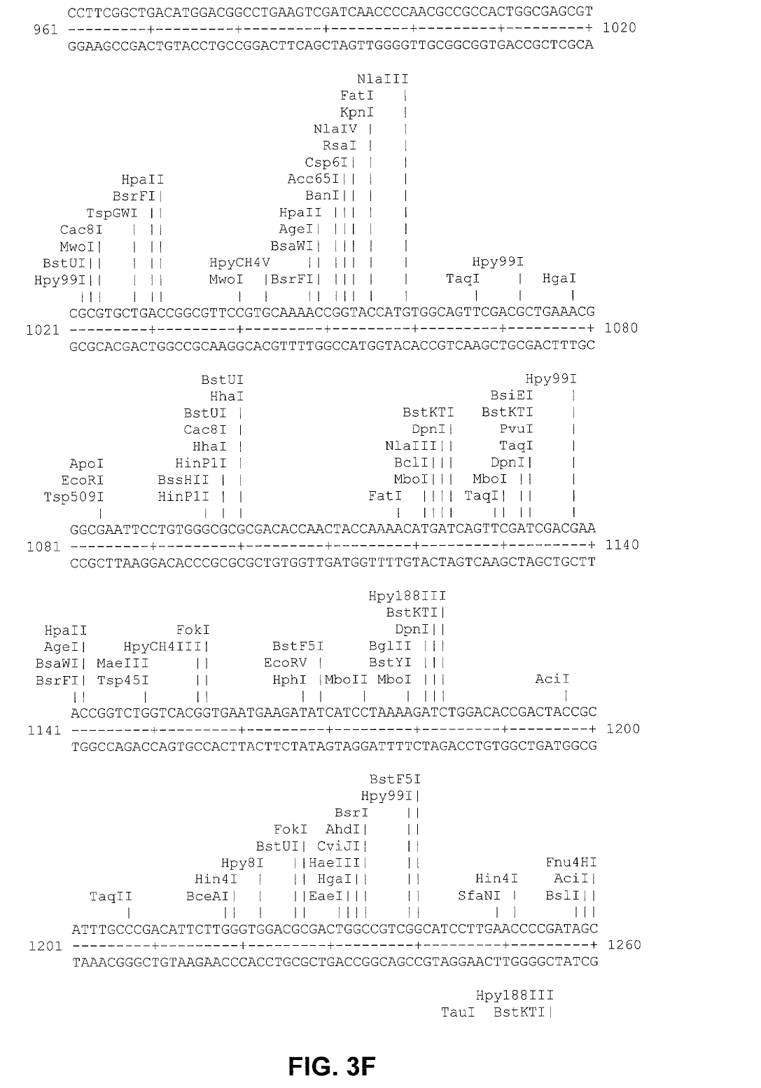

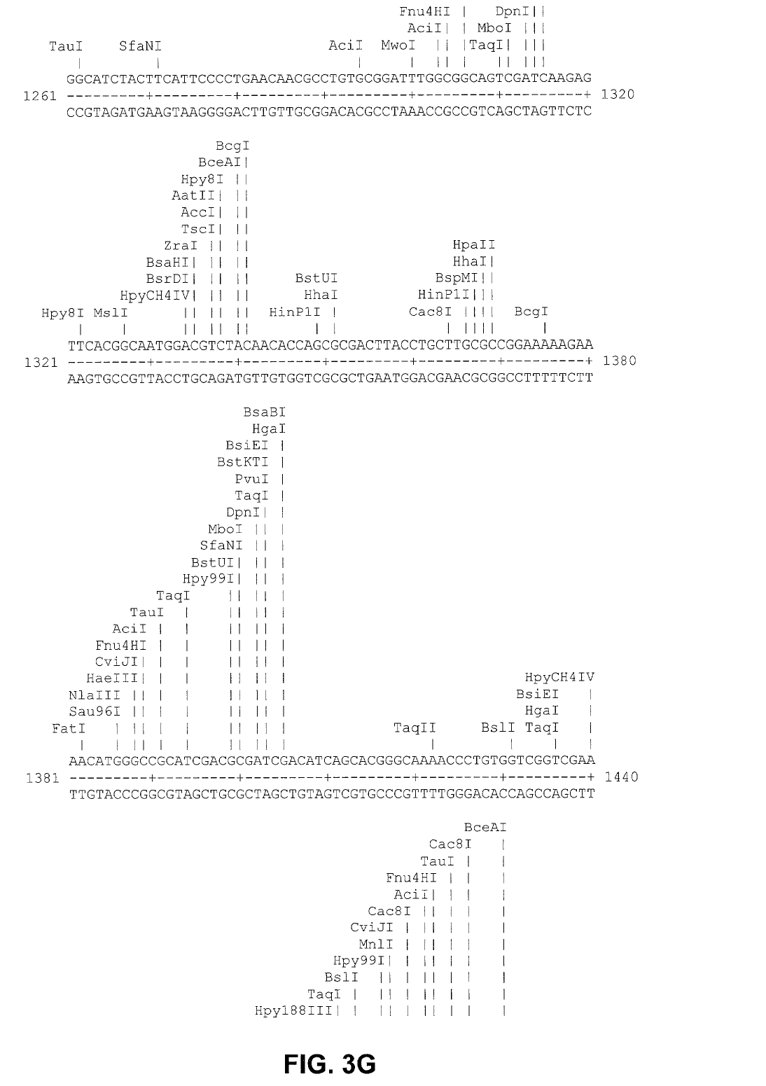

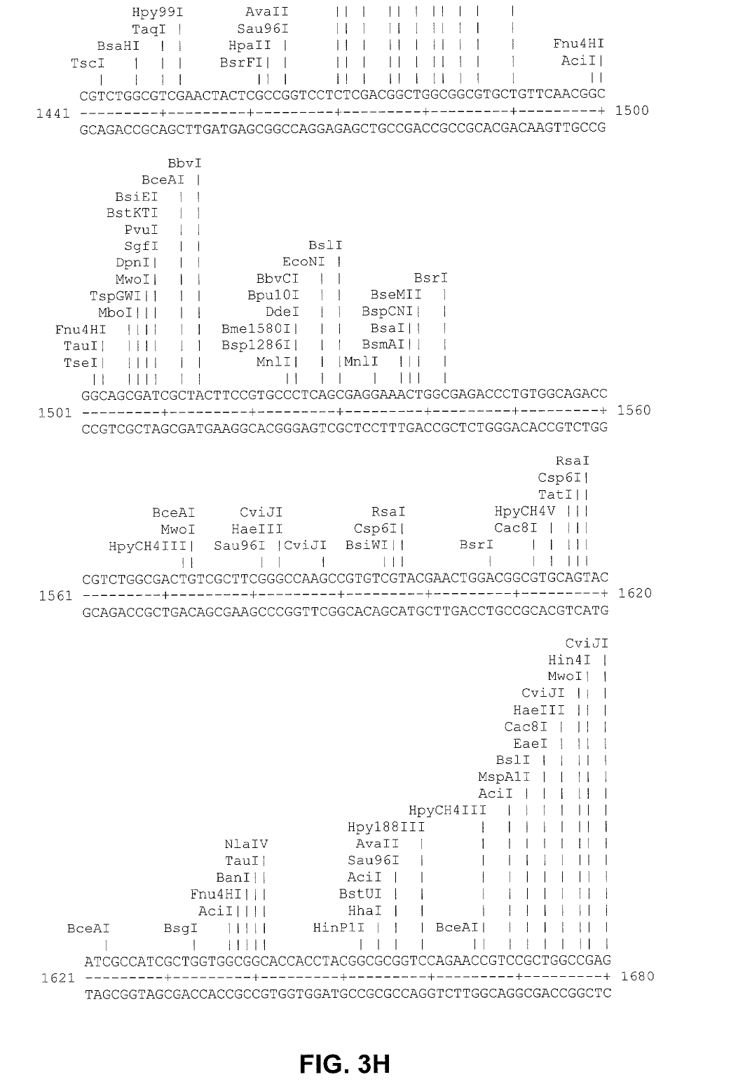

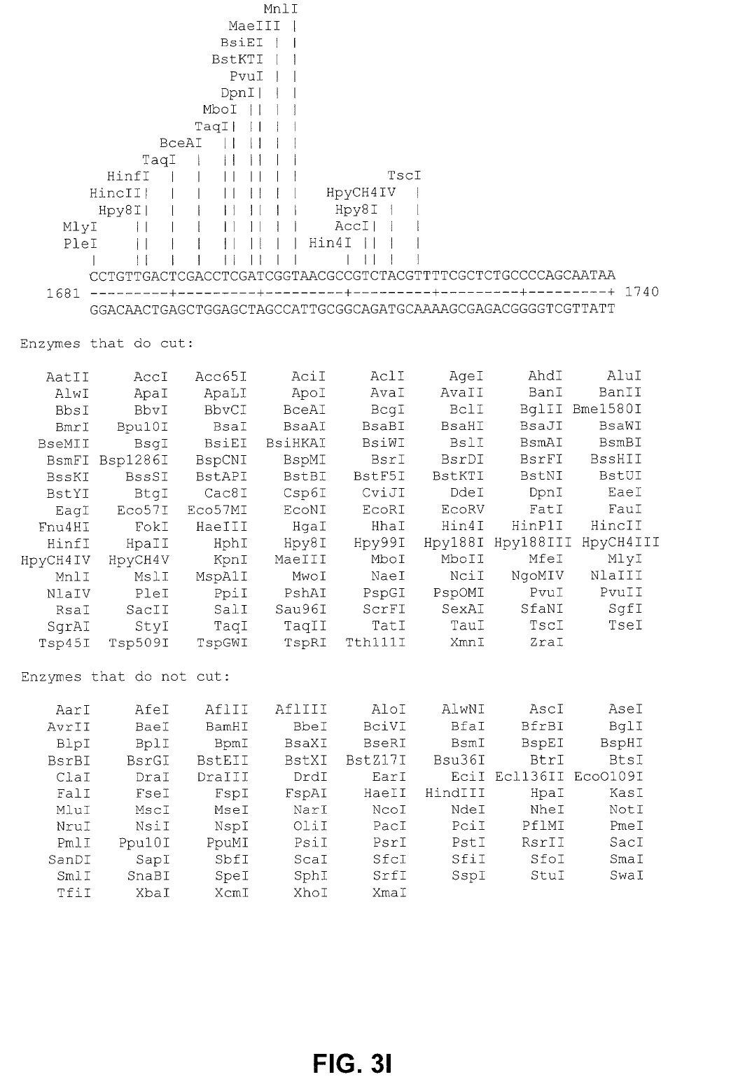

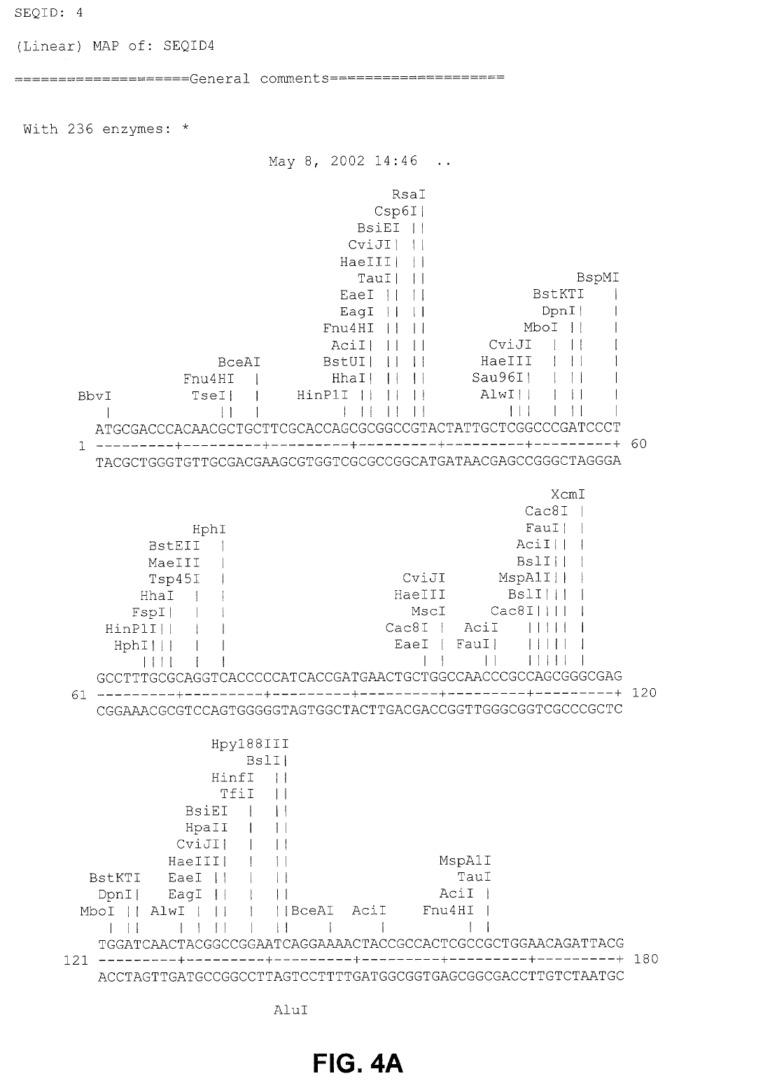

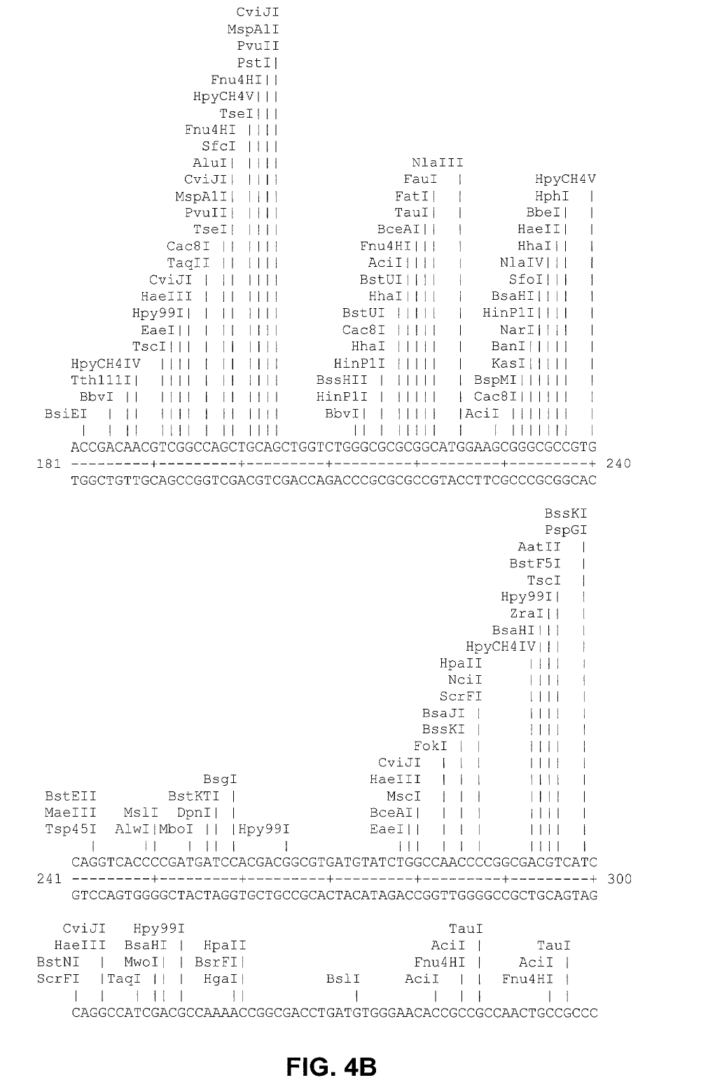

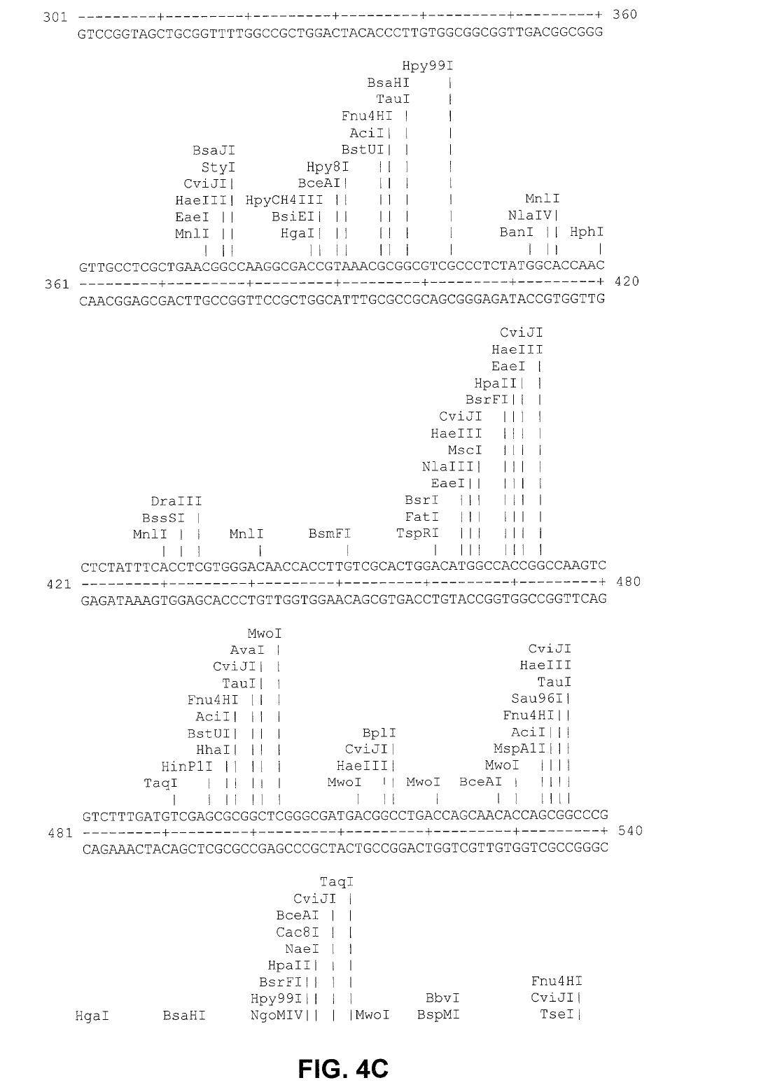

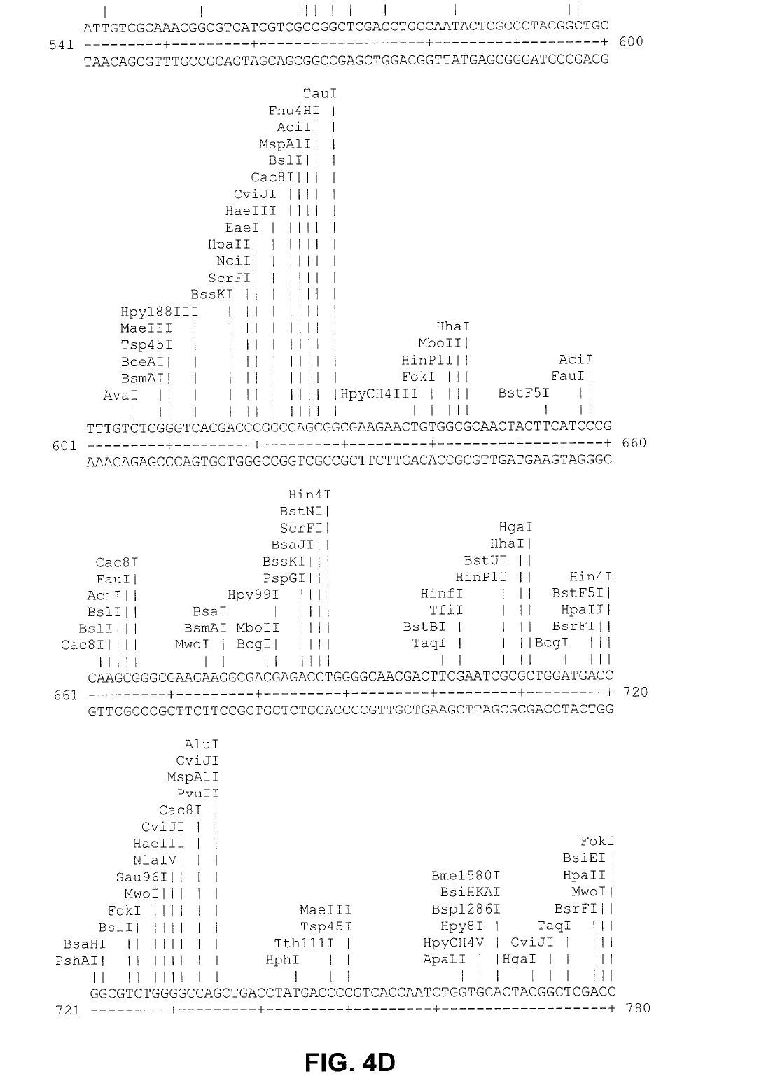

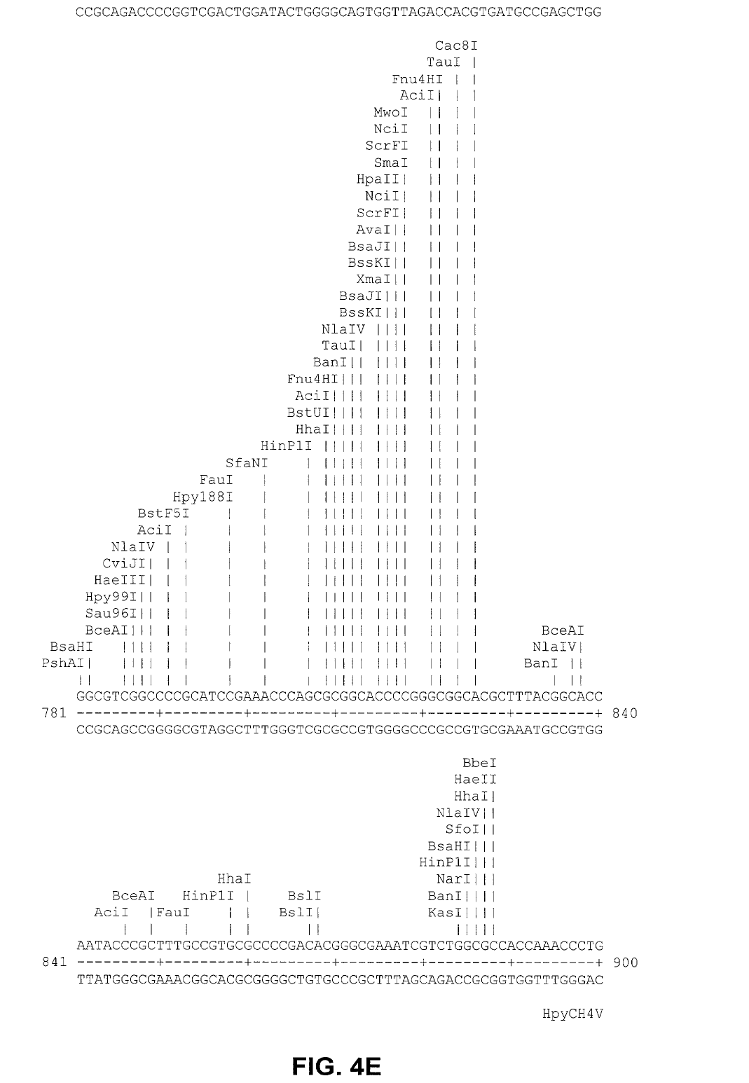

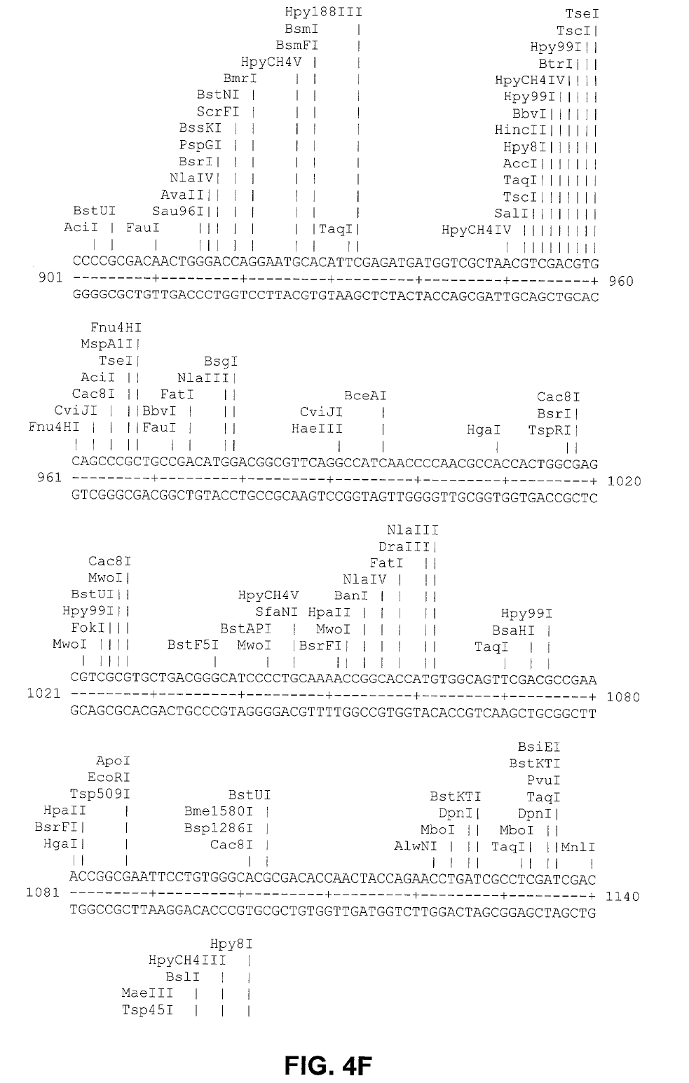

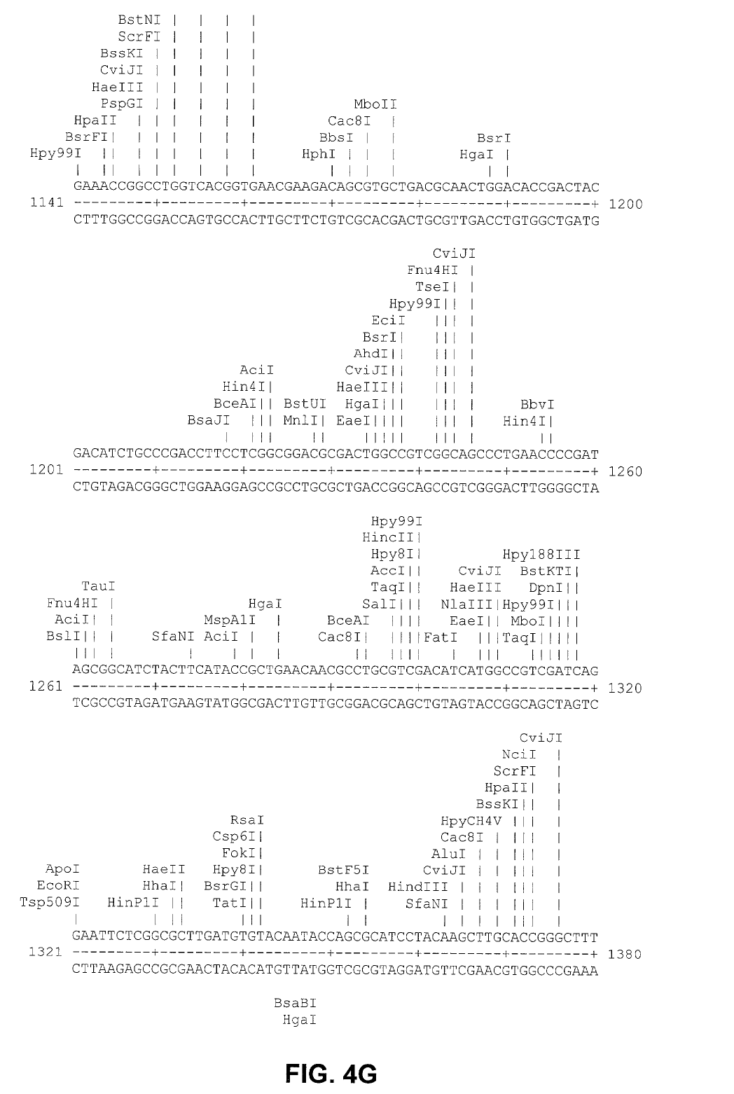

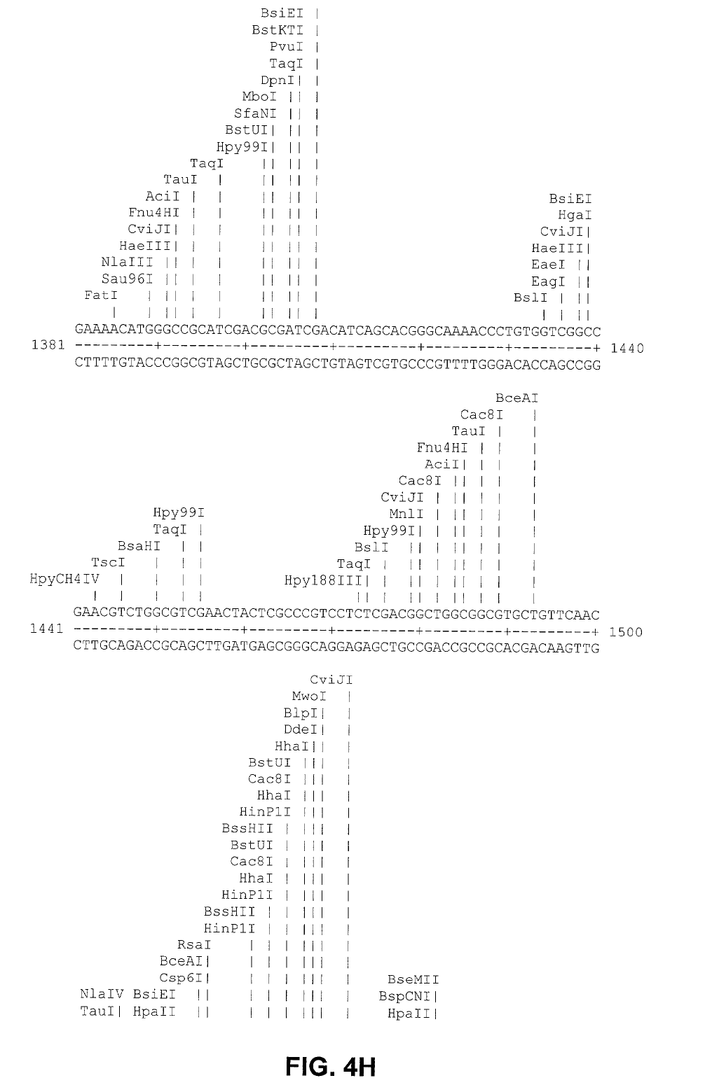

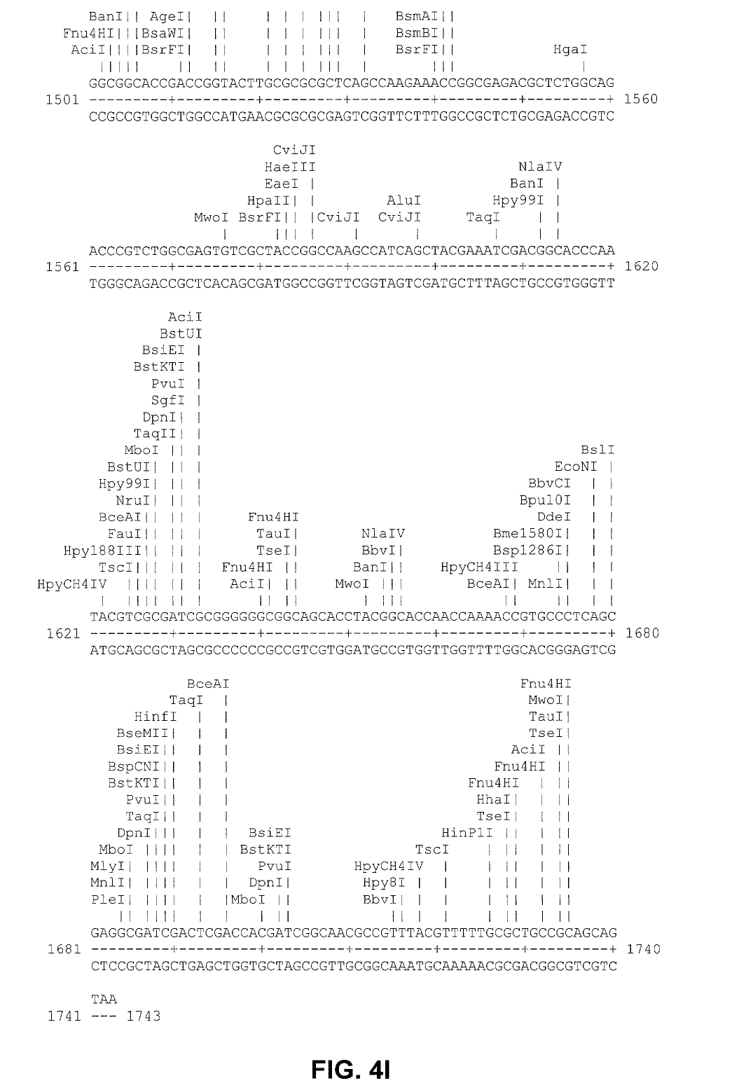

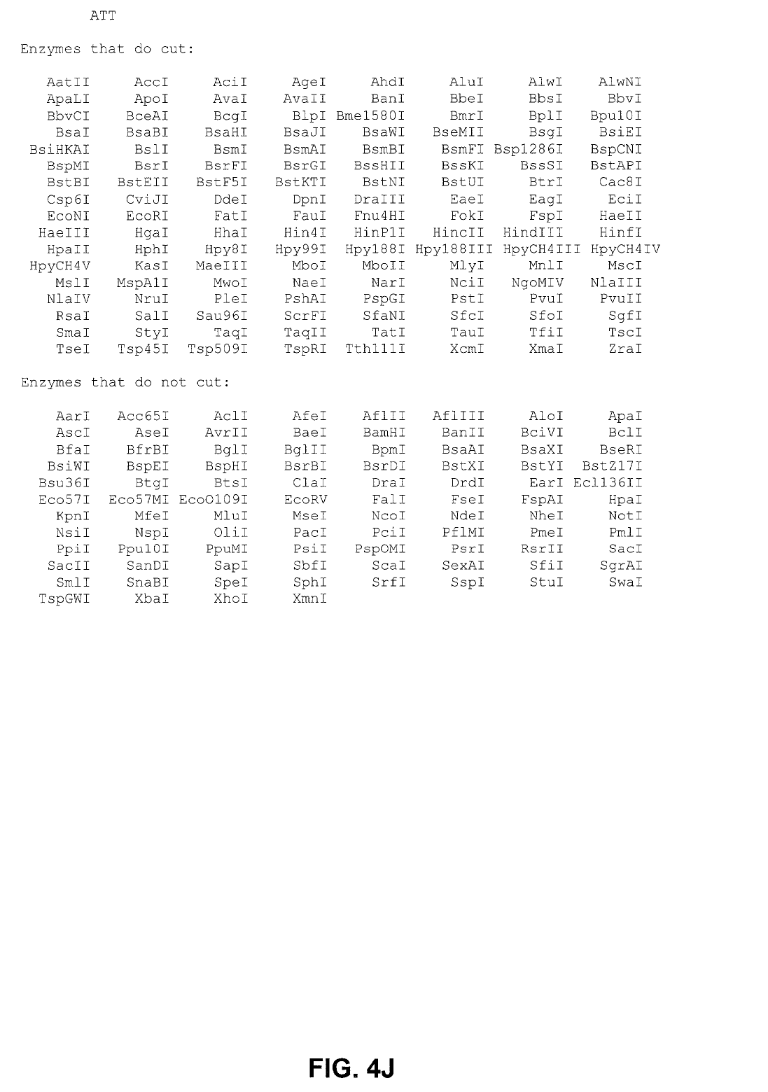

### DETAILED DESCRIPTION OF THE INVENTION

1. Definitions

Cloning Vector: A plasmid or phage DNA or other DNA sequence which is able to replicate autonomously in a host cell, and which is characterized by having restriction endonuclease recognition sites at which such DNA sequences may be cut, and into which a DNA fragment may be spliced in order to bring about its replication and cloning. The cloning vector may further contain a marker suitable for use in the identification of cells transformed with the cloning vector. Markers, for example, provide tetracycline resistance, ampicillin resistance, reversion to auxotrophy or other determinable characteristics.

Expression: Expression is the process by which a polypeptide is produced from a structural gene. The process involves transcription of the gene into mRNA and the translation of such mRNA into polypeptide(s).

Expression Vector: A vector similar to a cloning vector but which is capable of expressing a sequence that has been cloned into it, after transformation into a host. The cloned gene is usually placed under the control of (i.e., operably linked to) certain control sequences such as promoter sequences. Promoter sequences can be either constitutive, inducible or repressible.

Gene: A DNA sequence that contains information needed for expressing a polypeptide or protein.

Host: Any prokaryotic or eukaryotic cell that is the recipient of an external nucleic acid, especially a replicable expression vector or cloning vector. A “host,” as the term is used herein, also includes prokaryotic or eukaryotic cells that can be genetically engineered by well known techniques to contain desired gene(s) on its chromosome or genome. For examples of such hosts, see Sambrook et al., Molecular Cloning: A Laboratory Manual, Second Edition, Cold Spring Harbor Laboratory, Cold Spring Harbor, N.Y. (1989).

Homologous/Nonhomologous: Two nucleic acid molecules are considered to be “homologous” if their nucleotide sequences share a similarity of greater than 50%, as determined by HASH-coding algorithms (Wilber, W. J. and Lipman, D. J., Proc. Natl. Acad Sci. 80:726-730 (1983)). Two nucleic acid molecules are considered to be “nonhomologous” if their nucleotide sequences share a similarity of less than 50%

Mutation: As used herein, the term refers to base pair changes, insertions or deletions in the nucleotide sequence of interest.

Mutagenesis: As used herein, the term refers to a process whereby a mutation is generated in DNA. With “random” mutagenesis, the exact site of mutation is not predictable, occurring anywhere in the chromosome of the microorganism, and the mutation is brought about as a result of physical damage caused by agents such as radiation or chemical treatment.

Operon: As used herein, the term refers to a unit of bacterial gene expression and regulation, including the structural genes and regulatory elements in DNA.

Parental Strain: As used herein, the term refers to a strain of prokaryotic or eukaryotic microorganism subjected to some form of mutagenesis to yield the microorganism of the invention.

Phenotype: As used herein, the term refers to observable physical or biochemical characteristics dependent upon the genetic constitution of a microorganism and its environment.

Promoter: A DNA sequence generally described as a region upstream of the 5′ end of a gene, located proximal to the transcriptional start codon. The transcription of the operably linked gene(s) is initiated at the promoter region. If a promoter is an inducible promoter, then the rate of transcription increases in response to an inducing agent. In contrast, the rate of transcription is not regulated by an inducing agent if the promoter is a constitutive promoter. If a promoter is a repressible promoter, the rate of transcription is reduced in response to a repressing agent.

Recombinant Host: According to the invention, a recombinant host may be any prokaryotic or eukaryotic cell which contains the desired cloned genes or genetic construct on an expression vector or cloning vector. This term is also meant to include those prokaryotic or eukaryotic cells that have been genetically engineered to contain the desired gene(s) or genetic constructs in the chromosome or genome of that organism.

Recombinant vector: Any cloning vector or expression vector which contains the desired cloned gene(s) or genetic constructs.

2. Isolation and Characterization of the Sorbitol Dehydrogenases and Cytochrome c

The present invention isolates and purifies enzymes that catalyze the dehydrogenation of D-sorbitol from Ketogulonigenium robustum ADM X6L and Ketogulonigenium sp. ADM 291-19. These two strains can efficiently produce 2KLG from L-sorbose. Therefore, these strains are believed to follow the sorbosone pathway in 2KLG production. Thus, D-sorbitol is converted to L-sorbose by sorbitol dehydrogenase activity, L-sorbose to L-sorbosone by sorbose dehydrogenase activity and finally L-sorbosone to 2KLG by sorbosone dehydrogenase activity. These enzyme activities were localized to the periplasmic space and the enzymes were purified using column chromatography following the periplasm enrichment. Cytochrome c, which may serve as a natural electron acceptor for these dehydrogenases was also purified from the periplasmic fraction of the two strains. Biochemical properties of the purified enzymes are provided, as well as the determination of the N-terminal amino acid sequences of the purified enzymes and the cytochrome c using an amino acid sequence analyzer (Applied Biosystems, 477A).

The newly characterized enzymes are different from the reported sugar dehydrogenases involved in 2KLG production, particularly from the well-known sugar dehydrogenases of acetic acid bacteria, in that the new enzymes do not have extra subunits other than that which provides the dehydrogenase activity and that they have very broad substrate specificities. Thus, it was found that a single dehydrogenase was active against and could perform dehydrogenation of all three substrates, D-sorbitol, L-sorbose and glyoxal (an alternative substrate for L-sorbosone dehydrogenase). This enzyme was named sorbitol dehydrogenase, but the enzyme is equally active toward L-sorbose and glyoxal. In fact, the enzyme is highly active towards various alcohols, aldehydes, aldoses and ketoses.

The sorbitol dehydrogenase of the present invention may be isolated using standard protein techniques. Briefly, the localization of dehydrogenase activities toward D-sorbitol, L-sorbose and glyoxal were first determined with respect to soluble and membrane fractions of cell extracts. The enzyme activities for all three substrates were found exclusively in the soluble fraction in both strains, K. robustum ADM X6L and Ketogulonigenium sp. ADM 291-19. Next, the proteins in the periplasmic space and the cytoplasmic space were selectively enriched by osmotic shock fractionation and the enzyme activities were determined. Most of the enzyme activities were found in the periplsmic space.

Protein with dehydrognease activities toward D-sorbitol, L-sorbose and glyoxal was purified from the two strains. Cells of these two strains were obtained by 15 L of fermentation in tryptic soy broth medium supplemented with 1% each of D-sorbitol and L-sorbose. The periplasmic proteins were first prepared with enrichment by cold osmotic shock. The periplasmic proteins were then passed through DEAE-TSK columns and gel filtration columns (Superose 12). By this rather simple purification regime, essentially homogeneous preparations of the enzymes of the two strains could be obtained. The purified enzymes were analyzed by SDS-PAGE. The apparent molecular weight of the purified enzyme of K. robustum ADM X6L and Ketogulonigenium sp. ADM 291-19 was between 62,000 and 64,000 Daltons. It was noted that, in both cases, a single protein peak from DEAE-column showed the dehydrogenase activity toward all three substrates, D-sorbitol, L-sorbose and glyoxal.

Fractions of cytochrome c that were eluted from the DEAE column were further purified by rechromatography on a second DEAE column. The red fractions eluting from the second DEAE column were collected and analyzed by SDS-PAGE. Cytochrome c moved as a red band on the gel without any staining. The apparent molecular weight of this cytochrome c was about 15,000 Daltons for that from both K. robustum ADM X6L and Ketogulonigenium sp. ADM 291-19.

Using the purified SDH enzymes, the substrate specificity was determined. The purified enzymes were active toward D-sorbitol, L-sorbose and glyoxal. In fact, the SDH enzymes showed remarkably broad substrate specificity against various alcohols and aldehydes including sugar alcohols, aldoses, ketoses and aldonic acids.

Further details of the purification and characterization of the sorbitol dehydrogenases of the invention are provided in Example 1.

3. Cloning of Genes Encoding Sorbitol Dehydrogenases of K. robustum ADM 86-96

To obtain N-terminal amino acid sequence information of the purified SDHs, the purified proteins were subjected to SDS-PAGE and electroblotting onto a polyvinylidene difluoride (PVDF) membrane. After visualization with ponceau S stain, the section of membrane containing the SDH protein was applied to an amino acid sequence analyzer (Applied Biosystems, Model 477A).

N-terminal amino acid sequences of 25 and 9 residues were obtained for the SDHs of K. robustum ADM X6L and Ketogulonigenium sp. ADM 291-19, respectively (SEQ ID No:12 and 13, respectively). The cytochrome c of K. robustum ADM X6L was also subjected to the N-terminal amino acid sequencing and a sequence of 24 amino acid residues (SEQ ID No:14) could be obtained.

4. Genome Sequencing of K. robustum ADM 86-96

The entire genome of K. robustum ADM 86-96 (NRRL B-21630) has been sequenced using technology available from Integrated Genomics, Inc. K. robustum ADM 86-96 (NRRL B-21630) was deposited at the Agricultural Research Service Culture Collection (NRRL), 1815 North University Street, Peoria, Ill. 61604, USA, on Oct. 15, 1996 under the provisions of the Budapest Treaty and assigned accession number NRRL B-21630. In order to sequence a bacterial genome the following procedures are employed: DNA is extracted from the organism and random (i.e., normal fragment distribution) BAC, cosmid and plasmid gene libraries are constructed. The libraries are then sequenced by a combination of a “shot-gun” and primer steps, after which the genome is assembled. The genomes are completely sequenced, and gapped sequencing results in the revelation of over 98% of the available DNA information, including virtually every open reading frame (ORF).

Functions for “hypothetical proteins” can be predicted more accurately when taking into account other sets of data besides the usual ORF\protein sequence similarity. In order determine the genome, an approach is taken which encodes and incorporates the strain's biochemistry and the data on the functional clustering of ORFs into the genome analysis.

The N-terminal amino acid sequence of SDH enzyme of K. robustum ADM X6L (SEQ ID No:12) thus obtained was used in a search against the genome sequence database of K. robustum ADM 86-96 (NRRL B-21630) (a derivative of K. robustum ADM X6L obtained by chemical mutagenesis) for identification of coding gene(s). Search with SEQ ID No:12 identified two open reading frames (ORFs) showing complete matches to the SEQ ID No:12 from the genome sequence database. These two genes were named SDH1 and SDH2, respectively. In addition, another closely matching sequence was also found in the ORF and the gene was named SDH3.

N-terminal amino acid sequence of cytochrome c (SEQ ID No:14) from K. robustum ADM X6L identified a complete match in database. Interestingly, this ORF was found to locate just upstream of the ORF that codes for SDH2. In fact, the cytochrome c gene and the SDH2 gene constituted an operon. This operon structure strongly indicates that the cytochrome c may be the physiological electron acceptor for the dehydrogenase.

To clone the three SDH genes, SDH1, SDH2 and SDH3, PCR primers were synthesized based on the sequence information of the corresponding ORFs. Upstream primers for all three genes contained BamHI sites. The upstream primer for SDH2 gene contained NdeI site. Downstream primers for SDH1 and 2 contain HindIII site and the primer for SDH3 contains XhoI site. The three genes were cloned first by PCR and the PCR products were used as probe for Southern hybridization. PCR products for SDH1 and 3 were subcloned into a T-vector, which are linear, blunt-ended plasmids that contain several dT's added onto the vector by Taq polymerase, so as to be compatible with dA's added during a PCR reaction, and named pTSDH1 and 3, respectively. It was impossible to obtain PCR product for the entire operon that contains genes for both cytochrome c and SDH2. The SDH2 part could be amplified by PCR (pTSDH2) and, thus, used for a probe.

The Southern hybridization of HindIII or NotI digests of genomic DNA of K. robustum 86-96 (NRRL B-21630) was performed with the three PCR products as probes. When hybridized under a high stringency condition, three probes gave the same pattern of hybridization signals with slight different signal intensities of each signal for each probe. This indicates that there are at least three highly homologous SDH genes in K. robustum ADM 86-96 (NRRL B-21630).

pTSDH1 and 2 gave discrete signals at 12 kb region of HindIII digest and pTSDH3 gave a discrete signal at 3 kb NotI digest. The DNA of 12 kb HindIII fragment and 3 kb NotI fragment were eluted and mini-libraries were constructed. The libraries were screened again by Southern hybridization with the same probes to clone the genomic clones of SDH1, 2 and 3. By this procedure, genomic clones of SDH1 and 3 could be obtained and these were named pHdSDH1 and pNtSDH3, respectively. To reduce the size of pHdSDH1, 2.9 kb HindIII/StuI fragment was subcloned into HindIII/EcoRV site of pBluescript SK and named pSubSDH1. A genomic clone of SDH2 was difficult to obtain from 12 kb HindIII DNA library with pTSDH2 as probe. Therefore, a region containing cytochrome c gene of the SDH2 operon was amplified by PCR (pTCYP) and used for Southern hybridization. Southern hybridization with this probe gave a signal at 5 kb of BamHI/PstI digest. A mini-library was constructed with 5 kb BamHI/PstI fragments and the library was screened by Southern hybridization with pTCYP as probe. This way, a genomic clone of SDH2 was cloned and named pBPSDH2.

5. Nucleic Acid Molecules of the Invention

The invention provides isolated nucleic acid molecules encoding one or more of the SDH enzymes and the cytochrome c described herein. Methods and techniques designed for the manipulation of isolated nucleic acid molecules are well known in the art. For example, methods for the isolation, purification and cloning of nucleic acid molecules, as well as methods and techniques describing the use of eukaryotic and prokaryotic host cells and nucleic acid and protein expression therein, are described by Sambrook, et al., Molecular Cloning: A Laboratory Manual, Second Edition, Cold Spring Harbor, N.Y., 1989, and Current Protocols in Molecular Biology, Frederick M. Ausubel et al Eds., John Wiley & Sons, Inc., 1987, the disclosure of which is hereby incorporated by reference.

More particularly, the invention provides several isolated nucleic acid molecules encoding individual SDH enzymes and cytochrome c of the invention. Additionally, the invention provides several isolated nucleic acid molecules encoding one or more of the SDH enzymes and cytochrome c of the invention. For the purpose of clarity, the particular isolated nucleic acid molecules of the invention are described. Thereafter, specific properties and characteristics of these isolated nucleic acid molecules are described in more detail.

Unless otherwise indicated, all nucleotide sequences determined by sequencing a DNA molecule herein were determined using an automated DNA sequencer (such as the Model 373A from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “isolated” nucleic acid molecule is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

RNA vectors may also be utilized with the SDH nucleic acid molecules disclosed in the invention. These vectors are based on positive or negative strand RNA viruses that naturally replicate in a wide variety of eukaryotic cells (Bredenbeek, P. J. and Rice, C. M., Virology 3:297-310 (1992)). Unlike retroviruses, these viruses lack an intermediate DNA life-cycle phase, existing entirely in RNA form. For example, alpha viruses are used as expression vectors for foreign proteins because they can be utilized in a broad range of host cells and provide a high level of expression; examples of viruses of this type include the Sindbis virus and Semliki Forest virus (Schlesinger, S., TIBTECH 11:18-22 (1993); Frolov, I., et al., Proc. Natl. Acad. Sci. (USA) 93:11371-11377 (1996)).

As exemplified by Invitrogen's Sindbis expression system, the investigator may conveniently maintain the recombinant molecule in DNA form (pSinrep5 plasmid) in the laboratory, but propagation in RNA form is feasible as well. In the host cell used for expression, the vector containing the gene of interest exists completely in RNA form and may be continuously propagated in that state if desired.

In another embodiment, the invention further provides variant nucleic acid molecules that encode portions, analogs or derivatives of the isolated nucleic acid molecules described herein. Variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions.

Variants of the isolated nucleic acid molecules of the invention may occur naturally, such as a natural allelic variant. By an “allelic variant” is intended one of several alternate forms of a gene occupying a given locus on a chromosome of an organism. Genes II, Lewin, B., ed., John Wiley & Sons, New York (1985). Non-naturally occurring variants may be produced using art-known mutagenesis techniques.

Isolated nucleic acid molecules of the invention also include polynucleotide sequences that are 95%, 96%, 97%, 98% and 99% identical to the isolated nucleic acid molecules described herein. Computer programs such as the BestFit program (Wisconsin Sequence Analysis Package, Version 10 for Unix, Genetics Computer Group, University Research Park, 575 Science Drive, Madison, Wis. 53711) may be used to determine whether any particular nucleic acid molecule is at least 95%, 96%, 97%, 98% or 99% identical to the nucleotide sequences disclosed herein or the the nucleotides sequences of the deposited clones described herein. BestFit uses the local homology algorithm of Smith and Waterman, Advances in Applied Mathematics 2: 482-489 (1981), to find the best segment of homology between two sequences.

By way of example, when a computer alignment program such as BestFit is utilized to determine 95% identity to a reference nucleotide sequence, the percentage of identity is calculated over the full length of the reference nucleotide sequence and gaps in homology of up to 5% of the total number of nucleotides in the reference sequence are allowed. Thus, per 100 base pairs analyzed, 95% identity indicates that as many as 5 of 100 nucleotides in the subject sequence may vary from the reference nucleotide sequence.

The invention also encompasses fragments of the nucleotide sequences and isolated nucleic acid molecules described herein. In a preferred embodiment the invention provides for fragments that are at least 30 bases in length. The length of such fragments may be easily defined algebraically. For example, for an isolated nucleotide molecule that is 2,265 bases in length, a fragment (F1) of the sequence at least 30 bases in length may be defined as F1=30+X, wherein X is defined to be zero or any whole integer from 1 to 2,245. Similarly, fragments for other isolated nucleic acid molecules described herein may be defined in the same manner. As will be understood by those skilled in the art, the isolated nucleic acid sequence fragments of the invention may single stranded or double stranded molecules.

The invention discloses isolated nucleic acid sequences encoding three proteins having the SDH enzyme activities of this invention. Computer analysis provides information regarding the open reading frames, putative signal sequence and mature and/or processed protein forms. Genes encoding the SDH1 are contained in a 2.9 kb HindIII/StuI fragment of the pSubSDH1 clone, which is deposited as DNA under accession number KCTC 0913BP with the Korean Collection for Type Cultures (KCTC), Korea Research Institute of Bioscience and Biotechnology (KRIBB), 52, Oun-Dong, Yusong-Ku, Taejon 305-333, Republic of Korea. Genes encoding the cytochrome c and SDH2 are contained in a 5 kb BamHI/PstI fragment of the pBPSDH2 clone, which is deposited as DNA under accession number KCTC 0914BP with the Korean Collection for Type Cultures, Korea Research Institute of Bioscience and Biotechnology (KRIBB), 52, Oun-dong, Yusong-Ku, Taejon 305-333, Republic of Korea. Genes encoding the SDH3 are contained in a 3 kb Not I fragment of the pNtSDH3 clone, which is deposited as DNA under accession number KCTC 0915BP with the Korean Collection for Type Cultures (KCTC), Korea Research Institute of Bioscience and Biotechnology (KRIBB), 52, Oun-Dong, Yusong-Ku, Taejon 305-333, Republic of Korea. All deposits referred to herein were made on Dec. 14, 2000 in accordance with the Budapest Treaty, and all restrictions imposed by the depositor on the availability to the public of the deposited biological material will be irrevocably removed upon the granting of the patent.

Thus, the invention provides an isolated nucleic acid molecule contained in KCTC 0913BP. The invention also provides an isolated nucleic acid molecule contained in KCTC 0914BP. The invention further provides an isolated nucleic acid molecule contained in KCTC 0915BP.

The invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art and are commercially available. The following vectors are provided by way of example: Bacterial- pET (Novagen), pQE70, pQE60, pQE-9 (Qiagen), pBs, phagescript, psiX174, pBlueScript SK, pBsKS, pNH8a, pNH16a, pNH18a, pNH46a (Stratagene); pTrc99A, pKK223-3, pKK233-3, pDR540, pRIT5 (Pharmacia); and Eukaryotic- pWLneo, pSV2cat, pOG44, pXT1, pSG (Stratagene) pSVK3, pBPV, pMSG, pSVL (Pharmacia). Thus, these and any other plasmids or vectors may be used as long as they are replicable and viable in a host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In another embodiment, the invention provides processes for producing the vectors described herein which comprises: (a) inserting the isolated nucleic acid molecule of the invention into a vector; and (b) selecting and propagating said vector in a host cell.

Representative examples of appropriate hosts include, but are not limited to, bacterial cells, such as Gluconobacter, Brevibacterium, Corynebacterim, E. coli, Streptomyces, Salmonella typhimurium, Acetobacter, Pseudomonas, Pseudogluconobacter, Bacillus and Agrobacterium cells; fungal and yeast organisms including Saccharomyces, Kluyveromyces, Aspergillus and Rhizopus; insect cells such as Drosophila S2 and Spodoptera Sf9 cells; animal cells such as CHO, COS and Bowes melanoma cells; and plant cells. Appropriate culture mediums and conditions for the above-described host cells are known in the art.

6. Polypeptides of the Invention

The invention provides isolated polypeptide molecules for the SDH enzymes and the cytochrome c of the invention. Methods and techniques designed for the manipulation of isolated polypeptide molecules are well known in the art. For example, methods for the isolation and purification of polypeptide molecules are described Current Protocols in Protein Science, John E. Coligan et al. Eds., John Wiley & Sons, Inc. 1997, the disclosure of which is hereby incorporated by reference.

More particularly, the invention provides several isolated polypeptide molecules encoding the individual SDH enzymes and cytochrome c of the invention. For the purpose of clarity, the particular isolated polypeptide molecules of the invention are described. Thereafter, specific properties and characteristics of these isolated polypeptide molecules are described in more detail.

In one embodiment, the invention provides an isolated polypeptide comprising a polypeptide sequence selected from the group consisting of: (a) the polypeptide sequence encoded in the polynucleotide sequence of SEQ ID NO:1; (b) the polypeptide sequence of SEQ ID NO:5; and (c) a polypeptide at least about 10 amino acids long from the polypeptide sequence of SEQ ID NO:5.

In another embodiment, the invention provides an isolated polypeptide comprising a polypeptide sequence selected from the group consisting of: (a) the polypeptide sequence encoded in the polynucleotide sequence of SEQ ID NO:2; (b) the polypeptide sequence of SEQ ID NO:6; and (c) a polypeptide at least about 10 amino acids long from the polypeptide sequence of SEQ ID NO:6.

In yet another embodiment, the invention provides an isolated polypeptide comprising a polypeptide sequence selected from the group consisting of: (a) the polypeptide sequence encoded in the polynucleotide sequence of SEQ ID NO:3; (b) the polypeptide sequence of SEQ ID NO:7; and (c) a polypeptide at least about 10 amino acids long from the polypeptide sequence of SEQ ID NO:7.

In yet another embodiment, the invention provides an isolated polypeptide comprising a polypeptide sequence selected from the group consisting of: (a) the polypeptide sequence encoded in the polynucleotide sequence of SEQ ID NO:4; (b) the polypeptide sequence of SEQ ID NO:8; and (c) a polypeptide at least about 10 amino acids long from the polypeptide sequence of SEQ ID NO:8.

Other embodiments of the invention include an isolated polypeptide sequence comprising the polypeptide encoded by the isolated nucleic acid sequence SEQ ID NO:9; two isolated polypeptide sequences comprising the two polypeptides encoded by the isolated nucleic acid sequence SEQ ID NO:10; an isolated polypeptide sequence comprising the polypeptide encoded by the isolated nucleic acid sequence SEQ ID NO:11; an isolated polypeptide sequence comprising the polypeptide encoded by the DNA clone contained in KCTC Deposit No. 0913BP; two isolated polypeptide sequences comprising the two polypeptides encoded by the DNA clone contained in KCTC Deposit No. 0914BP; and an isolated polypeptide sequence comprising the polypeptide encoded by the DNA clone contained in KCTC Deposit No. 0915BP.

The term “isolated polypeptide” is used herein to mean a polypeptide removed from its native environment. Thus a polypeptide produced and/or contained within a recombinant host cell is considered isolated for purposes of the present invention. Also intended as an “isolated polypeptide” are polypeptides that have been purified, partially or substantially, from a recombinant host cell.

Polypeptides of the present invention include naturally purified products, products of chemical synthetic procedures, fusion proteins and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

The isolated polypeptides of the invention also include variants of those polypeptides described above. The term “variants” is also meant to include natural allelic variant polypeptide sequences possessing conservative or nonconservative amino acid substitutions, deletions or insertions. The term “variants” is also meant to include those isolated polypeptide sequences produced by the hand of man, through known mutagenesis techniques or through chemical synthesis methodology. Such man-made variants may include polypeptide sequences possessing convervative or non-conservative amino acid substitutions, detections or insertions.

Whether a particular amino acid is conservative or non-conservative is well known to those skilled in the art. Conservative amino acid substitutions do not significantly affect the folding or activity of the protein. For exemplary purposes Table 1 presents a list of conservative amino acid substitutions.

TABLE 1
Conservative Amino Acid Substitutions

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the protein of the present invention that are essential for function can be identified by methods known in the art, such as site-directed mutagenesis or alanine-scanning mutagenesis (Cunningham and Wells, Science, 244: 1081-1085 (1989)).

Isolated polypeptide molecules of the invention also include polypeptide sequences that are 95%, 96%, 97%, 98% and 99% identical to the isolated polypeptide molecules described herein. Computer programs such as the BestFit program (Wisconsin Sequence Analysis Package, Version 10 for Unix, Genetics Computer Group, University Research Park, 575 Science Drive, Madison, Wis. 53711) may be used to determine whether any particular polypeptide molecule is 95%, 96%, 97%, 98% or 99% identical to the polypeptide sequences disclosed herein or the polypeptide sequences encoded by the isolated DNA molecule of the deposited clones described herein. BestFit uses the local homology algorithm of Smith and Waterman, Advances in Applied Mathematics 2: 482-489 (1981), to find the best segment of homology between two sequences.

By way of example, when a computer alignment program such as BestFit is utilized to determine 95% identity to a reference polypeptide sequence, the percentage of identity is calculated over the full length of the reference polypeptide sequence and gaps in homology of up to 5% of the total number of amino acids in the reference sequence are allowed. Thus, per 100 amino acids analyzed, 95% identity indicates that as many as 5 of 100 amino acids in the subject sequence may vary from the reference polypeptide sequence.

The invention also encompasses fragments of the polypeptide sequences and isolated polypeptide molecules described herein. In a preferred embodiment the invention provides for fragments that are at least 10 amino acids in length. The length of such fragments may be easily defined algebraically. For example, for an isolated polypeptide molecule that is 754 amino acids in length, a fragment (F4) of the sequence at least 10 amino acids in length may be defined as F4=10+X, wherein X is defined to be zero or any whole integer from 1 to 744. Similarly, fragments for other isolated polypeptide molecules described herein may also be defined in the same manner.

7. Production of L-Sorbose and 2-Keto-L-Gulonic Acid

The invention provides processes for the production of L-sorbose and 2-keto-L-gulonic acid (2KLG), which are useful in the production of vitamin C.

The invention provides a method for the production of L-sorbose from D-sorbitol comprising: (a) transforming a host cell with at least one isolated nucleotide sequence selected from the group consisting of: (i) a polynucleotide encoding the amino acid sequence of SEQ ID NO:5; (ii) a polynucleotide encoding the amino acid sequence of SEQ ID NO:6; (iii) a polynucleotide encoding the amino acid sequence of SEQ ID NO:7; and (iv) a polynucleotide encoding the amino acid sequence of SEQ ID NO: 8; (b) selecting and propagating said transformed host cell; and (c) recovering L-sorbose.

The invention also provides a method for the production of L-sorbose from D-sorbitol comprising: (a) transforming a host cell with at least one isolated nucleotide sequence selected from the group consisting of: (i) a polynucleotide comprising the polynucleotide sequence of SEQ ID NO:1; (ii) a polynucleotide comprising the polynucleotide sequence of SEQ ID NO:2; (iii) a polynucleotide comprising the polynucleotide sequence of SEQ ID NO:3; and (iv) a polynucleotide comprising the polynucleotide sequence of SEQ ID NO:4; (b) selecting and propagating said transformed host cell; and (c) recovering L-sorbose.

Another aspect of the invention is drawn to a method for the production of 2KLG comprising: (a) transforming a host cell with at least one isolated nucleotide sequence selected from the group consisting of: (i) a polynucleotide encoding the amino acid sequence of SEQ ID NO:5; (ii) a polynucleotide encoding the amino acid sequence of SEQ ID NO:6; (iii) a polynucleotide encoding the amino acid sequence of SEQ ID NO:7; and (iv) a polynucleotide encoding the amino acid sequence of SEQ ID NO: 8; (b) selecting and propagating said transformed host cell; and (c) recovering 2KLG

Another aspect of the invention is drawn to a method for the production of 2KLG comprising: (a) transforming a host cell with at least one isolated nucleotide sequence selected from the group consisting of a polynucleotide comprising the polynucleotide sequence of SEQ ID NO:1; a polynucleotide comprising the polynucleotide sequence of SEQ ID NO:2; a polynucleotide comprising the polynucleotide sequence of SEQ ID NO:3; and a polynucleotide comprising the polynucleotide sequence of SEQ ID NO:4; (b) selecting and propagating said transformed host cell; and (c) recovering 2KLG.

The three SDH genes were firstly amplified in K. robustum strains by subcloning of the genes with their own promoters into the vector, pXH2, which was developed using a high-copy number endogenous plasmid from K. robustum ADM X6L as an E. coli-Ketogulonigenium shuttle vector. Usual protocols for electrotransformation for e.g. E. coli was far from optimal for K. robustum strains. Therefore, a new improved protocol was developed. These plasmids were transformed into the strains, K. robustum X6L and 86-96 (NRRL B-21630), by electroporation. In this case, gene dosage effect may be expected due to the apparent high copy number of the plasmid pXH2. The transformants were analyzed by HPLC for an improved conversion activity upon overexpression of each SDH gene using D-sorbitol or L-sorbose as substrate.

Overexpression of SDH1 and SDH3, especially SDH3, resulted in a significantly improved L-sorbose production from D-sorbitol, while overexpression of SDH2 significantly improved the 2KLG production from L-sorbose in flask scale experiments.

Other suitable bacteria for use as host cells in the processes provided herein for the production of L-sorbose and 2KLG are known to those skilled in the art. Such bacteria include, but are not limited to, Escherichia coli, Brevibacterium, Corynebacterium, Gluconobacter, Acetobacter, Erwinia, Pseudomonas, Pseudogluconobacter, Paracoccus, Rhodococcus, Roseobacter, and Rhodobacter.

Other host cells for expression of the SDH enzymes of the invention include: strains identified in U.S. Pat. No. 5,834,231; Ketogulonigenium robustum strains X6L^{TP} (NRRL B-21627 and KCTC 0858BP), 291-19^{PP} (NRRL B-30035), 266-13B^{PP} (NRRL B-30036) and 62A-12A^{pp} (NRRL B-30037) (Int. j. Syst. Evol. Microbiol. 51: 1059-1070 (2001)). Ketogulonigenium vulgare DSM 4025 (U.S. Pat. No. 4,960,695); Gluconobacter T100 (Appl. Environ. Microbiol. 63: 454-460 (1997)); Pseudogluconobacter saccharoketogenes IFO 14464 (European Patent No. 221 707); Pseudomonas sorbosoxidans (U.S. Pat. No. 4,933,289); and Acetobacter liquefaciens IFO 12258 (Appl. Environ. Microbiol. 61:413-420 (1995)).

In other embodiments of the invention, a variety of fermentation techniques known in the art may be employed in processes of the invention drawn to the production of L-sorbose and 2-keto-L-gulonic acid. Generally, L-sorbose and 2-keto-L-gulonic acid may be produced by fermentation processes such as the batch type or of the fed-batch type, or in immobilized cell systems. In batch type fermentations, all nutrients are added at the beginning of the fermentation. In fed-batch or extended fed-batch type fermentations one or a number of nutrients are continuously supplied to the culture, right from the beginning of the fermentation or after the culture has reached a certain age, or when the nutrient(s) which are fed were exhausted from the culture fluid. A variant of the extended batch of fed-batch type fermentation is the repeated fed-batch or fill-and-draw fermentation, where part of the contents of the fermenter is removed at some time, for instance when the fermenter is full, while feeding of a nutrient is continued. In this way a fermentation can be extended for a longer time.

Another type of fermentation, the continuous fermentation or chemostat culture, uses continuous feeding of a complete medium, while culture fluid is continuously or semi-continuously withdrawn in such a way that the volume of the broth in the fermenter remains approximately constant. A continuous fermentation can in principle be maintained for an infinite time.

Immobilized cell systems involve the inventive microorganism strain, or mutant or variant thereof, being contacted with L-sorbose for a sufficient time on a support as described infra, and then the accumulated 2-KLG is isolated. Preferably, the microorganism strain is cultivated in a natural or synthetic medium containing L-sorbose for a period of time for 2-KLG to be produced and the accumulated 2-KLG is subsequently isolated. Alternatively, a preparation derived from the cells of the microorganism strain may be contacted with L-sorbose for a sufficient time and the accumulated 2-KLG may then be isolated.

As used herein, “a preparation derived from the cells” is intended to mean any and all extracts of cells from the culture broths of the inventive strain or a mutant or variant thereof, acetone dried cells, immobilized cells on supports, such as polyacrylamide gel, κ-carrageenan and the like, and similar preparations. The accumulated 2-KLG may be isolated by conventional methods.

In a batch fermentation an organism grows until one of the essential nutrients in the medium becomes exhausted, or until fermentation conditions become unfavorable (e.g. the pH decreases to a value inhibitory for microbial growth). In fed-batch fermentations measures are normally taken to maintain favorable growth conditions, e.g. by using pH control, and exhaustion of one or more essential nutrients is prevented by feeding these nutrient(s) to the culture. The microorganism will continue to grow, at a growth rate dictated by the rate of nutrient feed. Generally a single nutrient, very often the carbon source, will become limiting for growth. The same principle applies for a continuous fermentation, usually one nutrient in the medium feed is limiting, all other nutrients are in excess. The limiting nutrient will be present in the culture fluid at a very low concentration, often unmeasurably low. Different types of nutrient limitation can be employed. Carbon source limitation is most often used. Other examples are limitation by the nitrogen source, limitation by oxygen, limitation by a specific nutrient such as a vitamin or an amino acid (in case the microorganism is auxotrophic for such a compound), limitation by sulphur and limitation by phosphorous.

In an alternative embodiment of the present invention, the inventive microorganism is cultivated in mixed culture with one or more helper strains. As used herein, “helper strain” is intended to mean a strain of a microorganism that increases the amount of 2-KLG produced in the inventive process. Suitable helper strains can be determined empirically by one skilled in the art. Illustrative examples of suitable helper strains include, but are not limited to, members of the following genera: Aureobacterium (preferably A. liquefaciens or A. saperdae), Corynebacterium (preferably C. ammoniagenes or C. glutamicum), Bacillus, Brevibacterium (preferably B. linens or B. flavum), Pseudomonas, Proteus, Enterobacter, Citrobacter, Erwinia, Xanthomonas and Flavobacterium. Preferably, the helper strain is Corynebacterium glutamicum ATCC21544.

The helper strain is preferably incubated in an appropriate medium under suitable conditions for a sufficient amount of time until a culture of sufficient population is obtained. This helper strain inoculum may then be introduced into the culture medium for production of 2-KLG either separately or in combination with the inventive microorganism strain, i.e., a mixed inoculum. Preferably, the ratio of the amount

Illustrative examples of suitable supplemental carbon sources include, but are not limited to: other carbohydrates, such as glucose, fructose, mannitol, starch or starch hydrolysate, cellulose hydrolysate and molasses; organic acids, such as acetic acid, propionic acid, lactic acid, formic acid, malic acid, citric acid, and fumaric acid; and alcohols, such as glycerol.

Illustrative examples of suitable nitrogen sources include, but are not limited to: ammonia, including ammonia gas and aqueous ammonia; ammonium salts of inorganic or organic acids, such as ammonium chloride, ammonium nitrate, ammonium phosphate, ammonium sulfate and ammonium acetate; urea; nitrate or nitrite salts, and other nitrogen-containing materials, including amino acids as either pure or crude preparations, meat extract, peptone, fish meal, fish hydrolysate, corn steep liquor, casein hydrolysate, soybean cake hydrolysate, soy molasses, yeast extract, dried yeast, ethanol-yeast distillate, soybean flour, cottonseed meal, and the like.

Illustrative examples of suitable inorganic salts include, but are not limited to: salts of potassium, calcium, sodium, magnesium, manganese, iron, cobalt, zinc, copper and other trace elements, and phosphoric acid.

Illustrative examples of appropriate trace nutrients, growth factors, and the like include, but are not limited to: coenzyme A, pantothenic acid, biotin, thiamine, riboflavin, flavine mononucleotide, flavine adenine dinucleotide, other vitamins, amino acids such as cysteine, sodium thiosulfate, p-aminobenzoic acid, niacinamide, soy molasses, and the like, either as pure or partially purified chemical compounds or as present in natural materials. Cultivation of the inventive microorganism strain may be accomplished using any of the submerged fermentation techniques known to those skilled in the art, such as airlift, traditional sparged-agitated designs, or in shaking culture.

The culture conditions employed, including temperature, pH, aeration rate, agitation rate, culture duration, and the like, may be determined empirically by one of skill in the art to maximize L-sorbose and 2-keto-L-gulonic acid production. The selection of specific culture conditions depends upon factors such as the particular inventive microorganism strain employed, medium composition and type, culture technique, and similar considerations.

Illustrative examples of suitable methods for recovering 2-KLG are described in U.S. Pat. Nos. 5,474,924; 5,312,741; 4,960,695; 4,935,359; 4,877,735; 4,933,289; 4,892,823; 3,043,749; 3,912,592; 3,907,639 and 3,234,105.

According to one such method, the microorganisms are first removed from the culture broth by known methods, such as centrifugation or filtration, and the resulting solution concentrated in vacuo. Crystalline 2-KLG is then recovered by filtration and, if desired, purified by recrystallization. Similarly, 2-KLG can be recovered using such known methods as the use of ion-exchange resins, solvent extraction, precipitation, salting out and the like.

When 2-KLG is recovered as a free acid, it can be converted to a salt, as desired, with sodium, potassium, calcium, ammonium or similar cations using conventional methods. Alternatively, when 2-KLG is recovered as a salt, it can be converted to its free form or to a different salt using conventional methods.

Methods used and described herein are well known in the art and are more particularly described, for example, in R. F. Schleif and P. C. Wensink, Practical Methods in Molecular Biology, Springer-Verlag (1981); J. H. Miller, Experiments in Molecular Genetics, Cold Spring Harbor Laboratory Press, Cold Spring Harbor, N.Y. (1972); J. H. Miller, A Short Course in Bacterial Genetics, Cold Spring Harbor Laboratory Press, Cold Spring Harbor, N.Y. (1992); M. Singer and P. Berg, Genes & Genomes, University Science Books, Mill Valley, Calif. (1991); J. Sambrook, E. F. Fritsch and T. Maniatis, Molecular Cloning. A Laboratory Manual, 2d ed., Cold Spring Harbor Laboratory Press, Cold Spring Harbor, N.Y. (1989); P. B. Kaufman et al., Handbook of Molecular and Cellular Methods in Biology and Medicine, CRC Press, Boca Raton, Fla. (1995); Methods in Plant Molecular Biology and Biotechnology, B. R. Glick and J. E. Thompson, eds., CRC Press, Boca Raton, Fla. (1993); P. F. Smith-Keary, Molecular Genetics of Escherichia coli, The Guilford Press, New York, N.Y. (1989); Plasmids: A Practical Approach, 2nd Edition, Hardy, K. D., ed., Oxford University Press, New York, N.Y. (1993); Vectors: Essential Data, Gacesa, P., and Ramji, D. P., eds., John Wiley & Sons Pub., New York, N.Y. (1994); Guide to Electroporation and electrofusions, Chang, D., et al., eds., Academic Press, San Diego, Calif. (1992); Promiscuous Plasmids of Gram-Negative Bacteria, Thomas, C. M., ed., Academic Press, London (1989); The Biology of Plasmids, Summers, D. K., Blackwell Science, Cambridge, Mass. (1996); Understanding DNA and Gene Cloning. A Guide for the Curious, Drlica, K., ed., John Wiley and Sons Pub., New York, N.Y. (1997); Vectors: A Survey of Molecular Cloning Vectors and Their Uses, Rodriguez, R. L., et al., eds., Butterworth, Boston, Mass. (1988); Bacterial Conjugation, Clewell, D. B., ed., Plenum Press, New York, N.Y. (1993); Del Solar, G., et al., “Replication and control of circular bacterial plasmids,” Microbiol. Mol. Biol. Rev. 62:434-464 (1998); Meijer, W. J., et al., “Rolling-circle plasmids from Bacillus subtilis: complete nucleotide sequences and analyses of genes of pTA1015, pTA1040, pTA1050 and pTA1060, and comparisons with related plasmids from gram-positive bacteria,” FEMS Microbiol. Rev. 21:337-368 (1998); Khan, S. A., “Rolling-circle replication of bacterial plasmids,” Microbiol. Mol. Biol. Rev. 61:442-455 (1997); Baker, R. L., “Protein expression using ubiquitin fusion and cleavage,” Curr. Opin. Biotechnol. 7:541-546 (1996); Makrides, S. C., “Strategies for achieving high-level expression of genes in Escherichia coli,” Microbiol. Rev. 60:512-538 (1996); Alonso, J. C., et al., “Site-specific recombination in gram-positive theta-replicating plasmids,” FEMS Microbiol. Lett. 142:1-10 (1996); Miroux, B., et al., “Over-production of protein in Escherichia coli: mutant hosts that allow synthesis of some membrane protein and globular protein at high levels,” J. Mol. Biol. 260:289-298 (1996); Kurland, C. G., and Dong, H., “Bacterial growth inhibited by overproduction of protein,” Mol. Microbiol. 21:1-4 (1996); Saki, H., and Komano, T., “DNA replication of IncQ broad-host-range plasmids in gram-negative bacteria,” Biosci. Biotechnol. Biochem. 60:377-382 (1996); Deb, J. K., and Nath, N., “Plasmids of corynebacteria,” FEMS Microbiol. Lett. 175:11-20 (1999); Smith, G. P., “Filamentous phages as cloning vectors,” Biotechnol. 10:61-83 (1988); Espinosa, M., et al., “Plasmid rolling cicle replication and its control,” FEMS Microbiol Lett. 130:111-120 (1995); Lanka, E., and Wilkins, B. M., “DNA processing reaction in bacterial conjugation,” Ann. Rev. Biochem. 64:141-169 (!995); Dreiseikelmann, B., “Translocation of DNA across bacterial membranes,” Microbiol. Rev. 58:293-316 (1994); Nordstrom, K., and Wagner, E. G., “Kinetic aspects of control of plasmid replication by antisense RNA,” Trends Biochem. Sci. 19:294-300 (1994); Frost, L. S., et al., “Analysis of the sequence gene products of the transfer region of the F sex factor,” Microbiol. Rev. 58:162-210 (1994); Drury, L., “Transformation of bacteria by electroporation,” Methods Mol. Biol. 58:249-256 (1996); Dower, W. J., “Electroporation of bacteria: a general approach to genetic transformation,” Genet. Eng. 12:275-295 (1990); Na, S., et al, “The factors affecting transformation efficiency of coryneform bacteria by electroporation,” Chin. J. Biotechnol. 11:193-198 (1995); Pansegrau, W., “Covalent association of the tral gene product of plasmid RP4 with the 5′-terminal nucleotide at the relaxation nick site,” J. Biol. Chem. 265:10637-10644 (1990); and Bailey, J. E., “Host-vector interactions in Escherichia coli,” Adv. Biochem. Eng. Biotechnol. 48:29-52 (1993).

All patents and publications referred to herein are expressly incorporated by reference. Having now generally described the invention, the same will be more readily understood through reference to the following Examples which are provided by way of illustration, and are not intended to be limiting of the present invention, unless specified.

### EXAMPLE 1

Isolation and Characterization of Sorbitol Dehydrogenases and Cytochrome c from Ketogulonigenium robustum ADM X6L and Ketogulonigenium sp. ADM 291-19

In crude extract of Ketogulonigenium robustum ADM X6L and Ketogulonigenium sp. ADM 291-19, three enzyme activities involved in the so-called sorbosone pathway for 2KLG production, namely, D-sorbitol dehydrogenase, L-sorbose dehydrognease and L-sorbosone dehydrogenase were detected. Enzymes that catalyze the dehydrogenation of D-sorbitol, L-sorbose and glyoxal were localized to the periplasm and purified from the periplasmic fraction of the two strains. Glyoxal served as an alternative for L-sorbosone that is not commercially available.

Cytochrome c, which may serve as natural electron acceptor for these dehydrogenases was also purified from the periplasmic fraction of the two strains.

Step 1: Cultivation of K. robustum ADM X6L and Ketogulonigenium sp. ADM 291-19

K. robustum ADM X6L and Ketogulonigenium sp. ADM 291-19 were inoculated into 5 ml of tryptic soy broth (17 g Bacto tryptone, 3 g Bacto soytone, 2.5 g glucose, 5 g sodium chloride and 2.5 g dipotassium phosphate per liter) and incubated at 30° C. for 24 hours. One milliliter (ml) each of these cultures were transferred to 50 ml of the same medium in a 500 ml flask and cultivated at 30° C. for 24 hours on a rotary shaker (180 rpm). Ten flask cultures thus prepared were used as an inoculum for a 30 L jar fermentor containing 15 L of the same medium containing extra carbon sources of 1% D-sorbitol and 1% L-sorbose and cultivated at 30° C. and pH 7.6 with 1 VVM of aeration to early stationary phase.

Step 2: Cellular Fractionation

Cells were harvested by centrifugation at 12,000 g for 10 min, washed once with 10 mM Tris·HCl buffer (pH 8.0) and disrupted by sonication in a Branson Sonifier 450 for 15 min at 50% duty cycle in ice. The homogenate thus prepared was centrifuged at 12,000 g for 5 min to remove the cell debris. The resulting supernatant was centrifuged at 100,000 g for 90 min to get the supernatant as soluble fraction and the precipitate as crude membrane fraction. The crude membrane fraction was solubilized with 1.5% n-octyl glucoside by stirring for 2 hours at 4° C. The resultant suspension was centrifuged at 12,000 g for 10 min to give a supernatant, designated as solubilized membrane fraction.

Step 3: Preparation of the Periplasmic Fraction

For more detailed localization study, periplasmic fraction was enriched by the method of osmotic shock (Neu, H. C. and Heppel, L. A., J. Biol. Chem., 240:3685 (1965)). Wet cells were resuspended at about 0.1 g/ml in 20% sucrose-0.03 M Tris·HCl buffer (pH 8.0) at room temperature. Di-sodium EDTA was added to 1 mM and the flask was shaken at 180 rpm at room temperature for 10 min. After centrifugation at 16,000 g for 20 min at 4° C., the supernatant was removed and the pellet was rapidly mixed with cold distilled water of the same volume as the original cell suspension and shaken in ice bucket in a shaker for 10 min. The mixture was centrifuged again to obtain the supernatant called “osmotic shock fluid”. The pellet was sonicated in the same buffer, centrifuged and the supernatant was obtained as “pellet sonicate”.

Step 4: Enzyme Activity Assay and Localization of Enzymes

The dehydrogenase enzyme activity was assayed spectrophotometrically using 2,6-dichlorophenol indophenol (DCIP) as an artificial electron acceptor and phenazine methosulphate (PMS) as an electron mediator. The reaction mixture contained 50 mM Tris·HCl buffer (pH 8.0), 10 mM MgCl₂, 5 mM CaCl₂, 5 mM KCN, 0.1 mM PMS, 0.12 mM DCIP, a substrate as D-sorbitol (250 mM), L-sorbose (250 mM) or glyoxal (50 mM) and enzyme solution in a total volumn of 1.0 ml. The rate of decrease in absorbance at 522 nm was determined. One unit of enzyme activity was defined as the amount of enzyme catalyzing reduction of 1 μmol of DCIP per minute. Since the presence of KCN in assay mixture with glyoxal as substrate resulted in an instantaneous discoloration of DCIP, KCN was omitted for assay with glyoxal as substrate.

Enzyme activity was determined also by the Ferric-Dupanol method (Wood, W. A. et al., Meth. Enzymol., 5, 287 (1962)). The enzyme was preincubated for 5 min at 25° C. The reaction was started by addition of 10 mM (final conc.) potassium ferricyanide and 250 mM (final conc.) D-sorbitol, L-sorbose, or glyoxal. After an appropriate time, the reaction was stopped by adding the ferric sulfate-Dupanol solution (Fe₂(SO₄)₃.nH₂O 5 g/l, Dupanol (sodium lauryl sulfate) 3 g/l and 85% phosphoric acid 95 ml/l) and the absorbance of the Prussian color developed was determined at 660 nm in a spectrophotometer.

Enzyme activities towards D-sorbitol, L-sorbose and glyoxal in different cellular fractions were determined for enzyme localization in K. robustum ADM X6L and Ketogulonigenium sp. ADM 291-19. In both cases, most of the enzyme activity detected in crude cell extract was found in the soluble fraction and the activity in solubilized membrane fraction was negligible when assayed with Ferric-Dupanol method. Typical distributions of enzyme activities are shown in Table 2.

TABLE 2

Substrate

| Fraction | D-sorbitol | L-sorbose | glyoxal |
|---|---|---|---|
| A. K. robustum ADM X6L | | | |
| Cell Free Extract | 0.92 | 0.64 | 0.39 |
| Soluble Fraction | 0.90 | 0.61 | 0.50 |
| Membrane Fraction | 0.01 | 0.01 | 0.00 |

*The enzyme activity was assayed with Ferric-Dupanol method and
expressed as ΔOD/min/4 μl enzyme.

| | | | |
|---|---|---|---|
| B. Ketogulonigenium sp. | | | |
| ADM 291-19 | | | |
| Cell Free Extract | 0.66 | 0.55 | 0.61 |
| Soluble Fraction | 0.65 | 0.53 | 0.52 |
| Membrane Fraction | 0.00 | 0.00 | 0.00 |

*The enzyme activity was assayed with Ferric-Dupanol method and expressed as ΔOD/min/2 μl enzyme.

These data indicate that the enzymes are exclusively located in the soluble fraction that includes both periplasm and cytoplasm. For further localization of the enzymes to periplasm or cytoplasm, periplasm was enriched by osmotic shock method as described in Step 3 of Example 1. In this method, the Tris-Sucrose wash and the osmotic shock fluid represent the periplasmic fraction and the pellet sonicate represents the cytoplasm and the cytoplasmic membrane. To ensure that this fractionation did not cause cell ruptures, the activity of the intracellular enzyme marker, β-galactosidase, was assayed for different fractions. As shown in Table 3 B, the release of the β-galactosidase from the cell was negligible. The SDH activities in different fractions (Table 3A) showed that a considerable amount of the enzyme activity was released into the extracellular medium upon osmotic shock of the cell. The remaining activity in pellet sonicate could be due to the incomplete release of periplasm by osmotic shock. Typical enzyme activity assay for SDH and β-galactosidase for fractions obtained by osmotic shock fractionation is shown in Table 3.

TABLE 3
A. SDH Activities*

| | Substrate | | |
|---|---|---|---|
| Fraction | D-sorbitol | L-sorbose | glyoxal |
| Tris-Sucrose Wash | 0.31 | 0.14 | 0.36 |
| Osmotic Shock Fluid | 0.45 | 0.20 | 0.43 |
| Pellet Sonicate | 0.68 | 0.43 | 0.76 |

*The enzyme activities were assayed with PMS-DCIP method and
expressed as ΔOD/min/50 μl enzyme.

B. β-Galactosidase Activity*

| | Activity |
|---|---|
| Fraction | Act.(nmol/min) |
| Tris-Sucrose Wash | 0.043 |
| Osmotic Shock Fluid | 0.057 |
| Pellet Sonicate | 2.2 |

*β-Galactosidase activity was assayed with the High Sensitivity β-Galactosidase Assay Kit (Stratagene) using chlorophenol red-β-D-galactopyranoside as substrate.

The enzyme assays for different substrates with different cellular fractions employing AND or NADP as cofactors did not show any sign of nicotinamide cofactor dependence in either strain.

Step 5: Purification by Chromatography

First, the periplasmic proteins from the cells of K. robustum ADM X6L and Ketogulonigenium sp. ADM 291-19 obtained in Step 1 of Example 1 were prepared by osmotic shock fractionation as described in Step 3 of Example 1. About 50 ml of osmotic shock fluid containing periplasmic proteins was loaded onto a DEAE-TSK 650 (S) (Merck) column (2.5×15 cm) previously equilibrated with 10 mM Tris·HCl buffer (pH 8.0). The column was first washed with 80 ml of 10 mM Tris·HCl buffer (pH 8.0) containing 0.1 M NaCl, followed by a gradient elution with 600 ml of the same buffer containing 0.1-0.5 M NaCl. Fractions containing 5 ml of eluant were collected and assayed for the SDH enzyme activities with D-sorbitol, L-sorbose and glyoxal as substrate. Fractions eluting at about 0.3 M NaCl showed enzyme activity. At about 0.15 M NaCl, eluted red fractions that probably contained cytochrome c. It was noted that, for both organisms, a single protein peak from DEAE column showed the dehydrogenase activities toward all three substrates.

Active fractions eluted from DEAE column were pooled and further purified by gel filtration column chromatography. The active fractions were concentrated by about 20 fold with ultrafiltration in Centricon 30 (Amicon). The concentrate was loaded onto Superose 12 (Pharmacia) and eluted isocratically with 50 mM sodium phosphate buffer (pH 7.0) containing 0.15 M NaCl. Eluting fractions were assayed for the enzyme activities and it was also noted that, for both organisms, a single protein peak from the column showed dehydrogenase activity toward all three substrates.

By this rather simple purification regime, essentially pure preparations of SDHs for both K. robustum ADM X6L and Ketogulonigenium sp. ADM 291-19 could be obtained as judged by the single bands on SDS-PAGE analysis. The apparent molecular weight of the purified enzymes of K. robustum ADM X6L and Ketogulonigenium sp. ADM 291-19 was between 62,000 and 64,000 as determined by SDS-PAGE.

Fractions of cytochrome c from DEAE column were pooled and re-chromatographed in the same column under the same conditions but with a shallower gradient (0.0-0.2 M NaCl). The red fractions eluting from the second DEAE column were analyzed by SDS-PAGE. The cytochrome c moved as a red band on the gel without any staining. The apparent molecular weight of the cytochrome c was about 15,000 for both K. robustum ADM X6L and Ketogulonigenium sp. ADM 291-19.

Using the purified SDH enzymes, the substrate specificity was determined and shown in Table 4. The purified enzymes were active toward D-sorbitol, L-sorbose and glyoxal. In fact, the SDH enzymes showed remarkably broad substrate specificity against various alcohols and aldehydes including sugar alcohols, aldoses, ketoses and aldonic acids.

TABLE 4

| | Strain | |
|---|---|---|
| | ADM X6L | ADM291-19 |
| Substrate | (% relative activity*) | (% relative activity*) |
| D-Sorbitol (250)** | 12.6 | 12.4 |
| L-Sorbose (250) | 8.7 | 8.8 |
| Glyoxal (250) | 15.1 | 16.1 |
| D-Glucose (250) | 27.6 | 20.0 |
| D-Gluconate (250) | 30.6 | 20.0 |
| Glycerol (250) | 56.5 | 32.5 |
| D-Fructose (250) | 8.0 | 11.7 |
| D-Mannitol (100) | 26.6 | 0 |
| Ethanol (50) | 20.0 | 38.0 |
| 1-Propanol (50) | 100 | 100 |

*Enzyme activity was measured by PMS-DCIP method.
**Figures in the parentheses denote substrate concentration in mM.

### EXAMPLE 2

Determination of the N-terminal Amino Acid Sequences of the SDHs

The purified SDHs prepared in Example 1 were subjected to SDS-PAGE (12.5% gel) and the separated proteins were electroblotted onto a polyvinylidene difluoride (PVDF) membrane (Bollag, D. M. and Edelstein, S. J., Protein Methods, Wiley-Liss, Inc., Chap 8 (1991)). After visualization with ponceau S stain, the section of membrane containing the SDH protein was cut into pieces and the membrane pieces were applied directly to an amino acid sequence analyzer (Applied Biosystems, Model 477A) for N-terminal amino acid sequence analysis.

The resultant data for the SDHs of K. robustum ADM X6L and Ketogulonigenium sp. ADM 291-19 (SEQ ID No:12 and 13, respectively) are shown in Table 5.

Table 5

| | |
|---|---|
| Sequence ID | |
| No. | N-terminal amino acid sequence |

| | |
|---|---|
| SEQ ID NO:12 | DVTPVTDELLANPPAGEWISYGGXNNX |
| SEQ ID NO:13 | QVTPVTDEL |

The cytochrome c of K. robustum ADM X6L was also subjected to the N-terminal amino acid sequencing and the resultant data (SEQ ID No:14) is shown in Table 6.

Table 6

| Sequence ID | |
|---|---|
| No. | N-terminal amino acid sequence |
| SEQ ID NO:14 | ADTAATEEAXATEGGTRTIYDGVF |

### EXAMPLE 3

Genome Sequencing of K. robustum ADM 86-96

The entire genome of K. robustum ADM 86-96 (NRRL B-21630) (a derivative of K. robustum ADM X6L obtained by chemical mutagenesis) has been sequenced using technology available from Integrated Genomics, Inc. In order to sequence a bacterial genome the following procedures are employed: DNA is extracted from the organism and random (i.e., normal fragment distribution) BAC, cosmid and plasmid gene libraries are constructed. The libraries are then sequenced by a combination of a “shot-gun” and primer steps, after which the genome is assembled. The genomes are completely sequenced, and gapped sequencing results in the revelation of over 98% of the available DNA information, including virtually every ORF.

Functions for “hypothetical proteins” can be predicted more accurately when taking into account other sets of data besides the usual ORF\protein sequence similarity. In order determine the genome, an approach is taken which encodes and incorporates the strain's biochemistry and the data on the functional clustering of ORFs into the genome analysis.

### EXAMPLE 4

Identification of Genes Encoding SDHs and Cytochrome c from Genome Sequence Database by the N-Terminal Amino Acid Sequence Information

N-terminal amino acid sequence of SDH enzyme of K. robustum ADM X6L (SEQ ID No:12) obtained from Example 2 was used in database search against the genome sequence database of K. robustum ADM 86-96 (NRRL B-21630) for identification of coding gene(s). Search with SEQ ID No:12 identified two open reading frames (ORFs) showing complete matches to the SEQ ID No:12, from the genome sequence database. These two genes were named SDH1 and SDH2, respectively. In addition, another closely matching sequence was also found in the ORF and the gene was named SDH3.

N-terminal amino acid sequence of cytochrome c from K. robustum ADM X6L (SEQ ID No:14) identified a complete matching ORF in the database. Interestingly, this ORF was found to locate just upstream of the ORF coding for SDH2. In fact, the cytochrome c gene and the SDH2 gene constituted an operon. This operon structure strongly indicates that the cytochrome c may be the physiological electron acceptor for the dehydrogenase.

The nucleotide sequences of the genes coding for the structural genes and their 5′- and 3′-flanking sequences including promoter sequences for the SDH1, SDH2, SDH3 and cytochrome c are shown in SEQUENCE LISTINGS. SEQ ID No:9 contains SDH1. SEQ ID NO:10 contains cytochrome c and SDH2 both of which constitute an operon. SEQ ID NO:11 contains SDH3.

The ORF coding for the structural gene of SDH1 has the nucleotide sequence of 1,737 bp (SEQ ID NO:1) and the amino acid sequence of 578 amino acids (SEQ ID NO:5). The structural gene is preceded by a Shine-Dalgarno sequence, “AGGA” positioned at 741-744 bp of SEQ ID NO:9. The 23 amino acid signal sequence positioned at 750-818 bp. The coding sequence of the mature part of the SDH1 protein positioned at 819-2,486 bp which encoded a 555 amino acid polypeptide whose derived N-terminal amino acid sequence was in perfect agreement with the amino acid residues obtained by N-terminal amino sequence analysis.

The ORF coding for the structural gene of cytochrome c has the nucleotide sequence of 495 bp (SEQ ID NO:2) and the amino acid sequence of 164 amino acids (SEQ ID NO:6). The gene is preceded by a Shine-Dalgarno sequence, “AGGA” positioned at 652-655 bp of SEQ ID NO:10. The 34 amino acid signal sequence of cytochrome c positioned at 663-764 bp. The coding sequence of the mature part of cytochrome c positioned at 765-1,157 bp which encoded a 130 amino acid polypeptide whose derived N-terminal amino acid sequence was in perfect agreement with the amino acid residues obtained by N-terminal amino sequence analysis. The ORF for cytochrome c was followed by the second ORF, the two ORF's being interrupted by a short intergenic region. The second ORF encodes SDH2. The ORF coding for the structural gene of SDH2 has the nucleotide sequence of 1,740 bp (SEQ ID NO:3) and the amino acid sequence of 579 amino acids (SEQ ID NO:7). The Shine-Dalgarno sequence “AGG” of SDH2 gene existed at 1,230-1,232 bp of SEQ ID NO:10. The 23 amino acid signal sequence of SDH2 positioned at 1,241-1,309 bp. The coding sequence of the mature part of the SDH2 protein positioned at 1,310-2,980 bp which encoded a 556 amino acid polypeptide whose derived N-terminal amino acid sequence was in perfect agreement with the amino acid residues obtained by N-terminal amino sequence analysis.

The ORF coding for the structural gene of SDH3 has the nucleotide sequence of 1,743 bp (SEQ ID NO:4) and the amino acid sequence of 580 amino acids (SEQ ID NO:8). The gene is preceded by a Shine-Dalgarno sequence, “AGGA” positioned at 521-524 bp of SEQ ID NO:11. The 23 amino acid signal sequence of SDH3 positioned at 530-598 bp. The coding sequence of the mature part of the SDH1 protein positioned at 599-2,272 bp which encoded a 557 amino acid polypeptide.

The calculated molecular weights of the mature proteins of the SDH1, SDH2 and SDH3 were 60,691, 60,639 and 60,403 Da, respectively, which are in good agreement with the experimental values of 62-64 kDa as determined by SDS-PAGE. The calculated molecular weight of the mature cytochrome c was 13,612 Da which is in good agreement with the experimental value of 15 kDa as determined by SDS-PAGE.

It was also found that the PQQ-binding signature sequences, consensus sequences appearing characteristically in PQQ-dependent dehydrogenases, existed in all three SDH genes. The signature sequence existing in amino-terminal part is shown as Sequence ID No.:23, and the signature sequence existing in the carboxy-terminal part is shown as Sequence ID No.:24 in Table 7 (Here, X represents an arbitrary amino acid).

Table 7

| | |
|---|---|
| [D/E/N]-W-X-X-X-G-[R/K]-X-X-X-X-X-X-[F/Y/W]-S-X-X-X-X-[L/I/V/M]-X-X-X- | [Sequence ID No.:23] |
| N-X-X-X-L-[R/K] | |
| W-X-X-X-X-Y-D-X-X-X-[D/N]-[L/I/V/M/F/Y]-[L/I/V/M/F/Y]-[L/I/V/M/F/Y]-[ | [Sequence ID No.:24] |
| L/I/V/M/F/Y]-X-X-G-X-X-[S/T/A]-P | |

In addition, it was discovered that a single heme-binding sequence of Sequence ID No.:25 in Table 8 below existed at 882-896 bp in cytochrome c gene of SEQ ID NO:10 (Here, X_{A} represents an arbitrary amino acid. X_{B} is an arbitrary amino acid different from X_{A}).

Table 8

| | |
|---|---|
| C-X_{A}-X_{B}-C-H | [Sequence ID No.:25] |

The three SDH genes shared considerable homologies in amino acid sequences each other. The similarities of SDH1 with SDH2 and SDH3 were 90.7 and 83.2%, respectively. The similarity of SDH2 and SDH3 was 82.1%.

### EXAMPLE 5

Cloning of the Genes Encoding Three SDHs of K. robustum ADM 86-96

The three SDH genes were cloned first by polymerase chain reaction (PCR) and the PCR products were used as probes in Southern hybridization to identify the corresponding genomic clones.

Step 1: Primer Design

To clone the three SDH genes, SDH1, SDH2 and SDH3, PCR primers were synthesized based on the sequence of corresponding ORFs. The primers were designed to include the original promoters of the genes in addition to the structural genes. Upstream primers for SDH1 (primer 1-1), cytochrome c (primer 2-1) and SDH3 (primer 3-1) genes contained BamHI site. The upstream primer for SDH2 (primer 2-3) gene contained NdeI site. Downstream primers for SDH1 (primer 1-3) and SDH2 (primer 2-4) contained HindIII site and the downstream primer for SDH3 (primer 3-3) contained XhoI site. The sequences of the primers are shown in Table 9 below.

Table 9

| Primer No. | SEQ ID NO | Sequence |
|---|---|---|
| 1-1 | 15 | GGATCCATACCTCGAGGTTGAAGC |
| 1-3 | 16 | AAGCTTGCGGTTTCCCGCGGGG |
| 2-1 | 17 | GGATCCTAGGCGAAAAGCCCCGCTT |
| 2-3 | 18 | CATATGAAGACGAAGTCTTTTCTG |
| 2-4 | 19 | AAGCTTATTGCTGGGGCAGAGCG |
| 3-1 | 20 | GGATCCCCACGCGAATAGCCCC |
| 3-3 | 21 | CTCGAGTTTTTACTGCTGCGGCAGC |

Step 2: PCR Cloning of the SDH Genes

The chromosomal DNA was isolated from K. robustum ADM 86-96 (NRRL B-21630) by Genomic-tip kit (Qiagen) and used as template for PCR. The three genes were cloned first by PCR and the PCR products were used as probe for Southern hybridization. Thirty cycles of polymerase chain reaction (1 min at 94° C., 1 min at 55° C., 3 min at 72° C. per cycle) were performed using a Premix PCR kit (Bioneer, Korea) for each pair of primers. PCR products of 2.3 kb and 2.26 kb for SDH1 and 3 were obtained with primers 1-1 and 1-3, and primers 3-1 and 3-3, respectively. These PCR products were gel-purified by QIAEX II Gel Extraction Kit (Qiagen), ligated into plasmid pT7 Blue (Novagen) and transformed into Escherichia coli DH5α by SEM method (Inoue, H. et al., Gene, 96:23 (1990)). Transformants were cultivated in an LB medium (1% Bacto-Tryptone, 0.5% yeast extract and 1% NaCl) supplemented with 100 μg/ml of ampicillin. Subsequently, the plasmid was extracted by alkaline lysis method (Sambrook, J. et al., Molecular Cloning, CSH Press, p. 125 (1988)). Clones with correct PCR product for SDH1 and SDH3 were selected by partial DNA sequencing and they were named pTSDH1 and pTSDH3, respectively. The primers, 2-1 and 2-4, used for cloning of the entire operon that contains genes for both cytochrome c and SDH2 repeatedly failed to produce PCR product. Therefore, the structural gene for SDH2 was first amplified by PCR using primers 2-3 and 2-4 for use as probe. The PCR product of 1.7 kb was gel-purified and subcloned into pT7 Blue to give pTSDH2.

Step 3: Southern Hybridization and Cloning of the Genomic SDH Clones

The Southern hybridization of genomic DNA of the strain, K. robustum ADM 86-96 (NRRL B-21630), was performed with the three PCR products as probe. The inserts from pTSDH1, pTSDH2 and pTSDH3 obtained in Step 2, Example 5, were isolated and labeled with DIG Labeling and Detection Kit (The DIG System User's Guide for Filter Hybridization. P. 6-9, Boehringer Mannheim (1993)). The chromosomal DNA from K. robustum ADM 86-96 (NRRL B-21630) prepared in Step 2, Example 5, was completely digested by HindIII or NotI and subjected to a 0.7% agarose gel electrophoresis. The DNA fragments separated on the gel were transferred onto a nylon membrane (NYTRAN, Schleicher & Schuell) as described (Southern, E. M., J. Mol. Biol. 98, 503 (1975)). The membrane was prehybridized in a hybridization oven (Hybaid) using a prehybridization solution (5× SSC, 1% (w/v) blocking reagent, 0.1% N-laurylsarcosine, 0.2% SDS and 50% (v/v) formamide) at 42° C. for 2 hours. Then the membrane was hybridized using a hybridization solution (DIG-labeled probe diluted in the prehybridization solution) at 42° C. for 12 hours. Southern hybridization gave strong discrete signals for each probe used. The three probes gave the same pattern of hybridization signals with slightly different signal intensities of each signal for each probe. This indicates that there are at least three highly homologous SDH genes in K. robustum ADM 86-96 (NRRL B-21630).

The two probes, pTSDH1 and 2, gave discrete and strong signals at 12 kb region of HindIII digest and pTSDH3 gave a strong discrete signal at 3 kb NotI digest. DNAs corresponding to the positive signals at 12 kb HindIII fragment and 3 kb NotI fragment were eluted and cloned into pBluescript SK (Stratagene) to construct mini-libraries. The mini-libraries were screened for positive clone by repeating the Southern hybridization as described above. By this procedure, the genomic clones for SDH1 and 3 could be obtained and these were designated pHdSDH1 and pNtSDH3, respectively. To reduce the size of pHdSDH1, 2.9 kb HindIII/StuI fragment containing SDH1 gene was subcloned into HindIII/EcoRV site of pBluescript SK and named pSubSDH1. Genomic clone for SDH2 was difficult to clone from 12 kb HindIII DNA library with pTSDH2 as probe. Therefore, a region containing a part of cytochrome c gene of SDH2 operon was amplified first by PCR for use as probe. A downstream primer 2-5 (5′-CCATGGCGGGAGTCCGCTCGATG-3′:SEQ ID NO:22) to span the 3′ sequence of the signal sequence of the cytochrome c was synthesized and used for PCR with the primer 2-1. The PCR product containing the promoter region of SDH2 operon and the signal sequence of the cytochrome c gene was obtained and subcloned into pT7 Blue to give rise to pTCYP. Southern hybridization with this probe gave a signal at 5 kb of BamHI/PstI digest. Mini-library was constructed with 5 kb BamHI/PstI fragment and screened by Southern hybridization with pTCYP as probe. A genomic clone of SDH2 operon was thus cloned and designated pBPSDH2.

### EXAMPLE 6

Construction of E. coli-K. robustum Shuttle Vector

Step 1. Isolation of Plasmid pXH2 Containing Linearized Ketogulonigenium Plasmid pADMX6L2

The DNA sequence of plasmid pADMX6L2 is about 4005 bp long and contains a single HindIII restriction site. The HindIII site was utilized to clone the pADMX6L2 sequence into the E. coli vector pUC19. A DNA prep containing a mixture of the endogenous plasmids from Ketogulonigenium robustum ADMX6L, and purified pUC 19 DNA from E. coli, were made and separately digested with restriction enzyme HindIII. A Wizard DNA Clean-Up kit was used to separate the digested DNA from enzyme and salts in preparation for the next step, with a final DNA suspension volume of 50 μl. DNA from the two digestions (3 μl of pJND1000 DNA and 10 μl of Ketogulonigenium plasmid DNA) was mixed and ligated overnight at room temperature using T4 ligase with the protocol of GibcoBRL technical bulletin 15244-2 (Rockville, Md.). Strain E. coli DH5αMCR was transformed with the ligation mixture following an established protocol (“Fresh Competent E. coli prepared using CaCl₂”, pp. 1.82-1.84, in J. Sambrook, E. F. Fritsch and T. Maniatis, Molecular Cloning: A Laboratory Manual, 2nd Ed. (1989)). Transformants were plated on LB agar plates containing 100 μg/ml of ampicillin and 40 μg/ml of Xgal and grown at 37° C. Plasmid DNA from transformants giving white colonies was isolated and digested with HindIII and NdeI, giving the expected fragment sizes for correct insertion of linearized pADMX6L2 DNA into the pUC19 vector. The identity of the pADMX6L2 insert was further confirmed by partial DNA sequencing into the pADMX6L2 region using the M13 primer regions of pUC19. The chimeric plasmid containing linearized pADMX6L2 cloned into pUC19 was named pXH2. An E. coli host transformed with pXH2 was deposited in the patent collection of the National Regional Research Laboratories in Peoria, Ill., U.S.A. under the terms of the Budapest Treaty as NRRL B-30419.

Step 2. Construction of E. coli/Ketogulonigenium Shuttle Plasmid pXH2/K5

Since kanamycin resistance can be expressed in Ketogulonigenium, it was desirable to replace the amp^{R} resistance gene in pXH2 with a kanamycin resistance gene, thereby allowing the selective isolation of Ketogulonigenium strains transformed with a plasmid. In vitro transposition was used to move a kanamycin resistance gene into pXH2 and simultaneously inactivate ampicillin resistance. Insertion of a kanamycin resistance gene into the ampicillin resistance gene of pXH2 was achieved using Epicentre technologies EZ::TN Insertion System (Madison, Wis.). 0.05 pmoles of pXH2 was combined with 0.05 pmoles of the <KAN-1> Transposon, 1 μl of EZ::TN 10X Reaction Buffer, 1 μl of EZ::TN Transposase, and 4 ul of sterile water giving a total volume of 10 μl in the transposition reaction. This mixture was incubated at 37° C. for 2 hours, then stopped by adding 1 μl of EZ::TN 10X Stop Solution, mixing, and incubation at 70° C. in a heat block for 10 minutes. E. coli DH5αMCR was transformed with the DNA mixture and transformants were selected on Luria Broth agar plates containing 50 μg/ml of kanamycin. Colonies recovered from these plates were patched onto two LB agar plates, one with 50 μg/ml of kanamycin and the other with 100 μg/ml of ampicillin. Only those that grew on the kanamycin plates were saved. Plasmid DNA from an ampicillin sensitive, kanamycin resistant colony was isolated as “pXH2/K5”. The proper insertion of the kanamycin resistance transposon into the vector ampicillin resistance gene was confirmed by analyzing pXH2/K2 DNA with various restriction endonucleases. To demonstrate the viability of pXH2/K5 as an E. coli/Ketogulonigenium shuttle vector, Ketogulonigenium robustum strain ADMX6L was transformed with pXH2/K5 DNA using the electroporation technique of Example 2. Stable, kanamycin resistant transformants were obtained from which pXH2/K5 DNA can be reisolated.

### EXAMPLE 7

Electrotransformation of K. robustum ADM X6L and K. robustum ADM 86-96

Usual protocols for electrotransformation for e.g. E. coli was far from optimal for K. robustum strains. Therefore, a new improved protocol was developed.

For preparation of competent cells for electroporation, cells of K. robustum were grown in 5 ml RM medium (10 g Bacto soytone, 10 g yeast extract, 5 g malt extract, 5 g NaCl, 2.5 g K₂HPO₄ and 20 g mannitol per liter, pH adjusted to 8.0) overnight. One ml of this culture was used to seed 50 ml of RM in a 500 ml flask. Cells were grown until the culture reaches an OD₆₀₀ of 1.0 and harvested by centrifugation for 5 min at 12,000 rpm in a SS-34 rotor in a Sorvall RC-5C centrifuge. The pellet was washed two times with deionized water and resuspended in 4 ml DW containing 280 μl DMSO. About 200 μl of the cell suspension was aliquoted and stored in a liquid nitrogen tank.

For electroporation, about 10 μl plasmid DNA (˜1 μg/ml) was mixed with 200 μl competent cells in a 2 mm cuvette (BioRad) and incubated for 5 min at 30° C. Electrical pulse was applied at 10 kV/cm, 600 Ω and 25 μF. Immediately after electric shock, 1 ml RM medium was added to the cuvette, the mixture was transferred to a 10 ml glass culture tube and incubated with shaking at 200 rpm for 3 hours at 30° C. The mixture was spread on RM plate containing 20 μg/ml Kanamycin for 2-3 days at 30° C.

With this new protocol, transformation efficiencies of up to ˜200 CFU/μg DNA could be obtained for K. robustum ADM X6L.

### EXAMPLE 8

Amplification of SDH Genes in K. robustum ADM X6L and 86-96

For expression of the three SDH genes in K. robustum strains, the genes with their own promoters were subcloned into the vector, pXH2/K5. These plasmids were transformed into the strains, K. robustum ADM X6L and 86-96 (NRRL B-21630), by electroporation. In this case, gene dosage effect may be expected due to the apparent high copy number of the plasmid pXH2/K5. The transformants were analyzed by HPLC for improved conversion activity upon overexpression of each SDH gene using D-sorbitol and L-sorbose as substrate.

For expression study, it is important to have faithfully amplified genes. Therefore, the incorporation of the part generated by PCR was minimized by replacement of the major part of the PCR products with the corresponding part from genomic clones. An NheI/EcoNI fragment of 1.43 kb containing most of the SDH1 structural gene was isolated from the genomic clone, pSubSDH1. This fragment was used to replace the same fragment in the PCR clone, pTSDH1, to give pRSDH1. A PstI/EcoNI fragment of 1.48 kb containing most of the SDH3 structural gene was isolated from the genomic clone, pNtSDH3, and used for replacement of the same fragment in pTSDH3 to give pRSDH3. For overexpression of SDH1 in K. robustum strains, a HindIII fragment of 2.30 kb was isolated from pRSDH1, made blunt by Klenow and subcloned into SmaI site of pXH2/K5. The resulting plasmid was named pXSDH1 and used for transformation of the K. robustum strains. For overexpression of SDH3 in K. robustum strains, a BamHI fragment of 2.29 kb was isolated from pRSDH3, made blunt by Klenow and subcloned into SmaI site of pXH2/K5. The resulting plasmid was named pXSDH3 and used for transformation of the K. robustum strains.

In the case of SDH2 operon, an AvrII/PstI fragment of 3.4 kb containing the promoter, the cytochrome c structural gene and the SDH2 structural gene was isolated from the genomic clone, pBPSDH2. This fragment was made blunt-ended by Klenow and subcloned into SmaI site of pXH2/K5. The resulting plasmid was designated pXSDH2 and used for transformation of K. robustum strains.

The effect of overexpression of the three SDH genes in bioconversion activity was analyzed by HPLC analysis of the product profile of the culture supernatant of transformants in a medium containing D-sorbitol or L-sorbose as substrate. The transformants were cultivated overnight in RM medium containing 20 μg/ml of Kanamycin. This overnight culture was used to inoculate the 25 ml RM medium (supplemented with 20 μg/ml of Kanamycin) containing 20 g/l of D-sorbitol or 20 g/l of L-sorbose as substrate instead of mannitol. The culture supernatant samples were taken in a 12-hour interval and were analyzed by HPLC (Gilson HPLC with RI detector (ERC-7515A, ERC Inc.)) equipped with Asted XL automatic sample injector. Samples were loaded on two 2 mm×300 mm×7.8 mm Aminex HPX-87H columns (BioRad) arranged in series to provide a total column length of 600 mm. The column was run at 50° C. at a flow rate of 0.5 ml/min. The peaks were identified and the concentration was determined by comparison with those of the standards using glycerol as internal standard.

K. robustum ADM X6L transformants harboring the pXSDH1, pXSDH2 or pXSDH3 were cultivated with D-sorbitol or L-sorbose as substrate. HPLC analysis of the products from D-sorbitol (Table 10) or L-sorbose (Table 11) is shown below. A transformant harboring pXH2 served as control.

TABLE 10

| | L-sorbose | 2KDG* | 2KLG** |
|---|---|---|---|
| pXH2 | | | |
| 12 hr | 0.8 | 2.0 | 1.2 |
| 24 hr | 1.6 | 3.1 | 1.7 |
| 36 hr | 2.0 | 3.7 | 2.3 |
| 48 hr | 2.1 | 4.2 | 2.7 |
| pXSDH1 | | | |
| 12 hr | 1.7 | 1.5 | 0.0 |
| 24 hr | 5.9 | 1.9 | 1.3 |
| 36 hr | 5.6 | 1.9 | 1.5 |
| 48 hr | 6.2 | 2.3 | 1.7 |
| pXSDH2 | | | |
| 12 hr | 1.8 | 3.2 | 1.0 |
| 24 hr | 1.8 | 5.1 | 2.3 |
| 36 hr | 1.0 | 5.8 | 2.7 |
| 48 hr | 0.8 | 5.3 | 2.4 |
| pXSDH3 | | | |
| 12 hr | 2.9 | 1.2 | 0.0 |
| 24 hr | 8.1 | 1.6 | 1.6 |
| 36 hr | 8.4 | 1.7 | 2.4 |
| 48 hr | 8.9 | 1.9 | 2.0 |

*2KDG denotes 2-keto-D-gluconic acid.
**2KLG denotes 2-keto-L-gulonic acid.

TABLE 11

| | 2KLG |
|---|---|
| pXH2 | |
| 12 hr | 0.6 |
| 24 hr | 2.9 |
| 36 hr | 3.6 |
| 48 hr | 3.4 |
| pXSDH1 | |
| 12 hr | 0.2 |
| 24 hr | 1.2 |
| 36 hr | 1.0 |
| 48 hr | 1.7 |
| pXSDH2 | |
| 12 hr | 1.8 |
| 24 hr | 7.4 |
| 36 hr | 9.1 |
| 48 hr | 11.5 |
| pXSDH3 | |
| 12 hr | 0.0 |
| 24 hr | 1.1 |
| 36 hr | 1.4 |
| 48 hr | 1.3 |

As shown in Table 10, overexpression of SDH1 and SDH3, especially SDH3, resulted in a significantly improved L-sorbose production (c.a. 2 and 3 times, respectively) from D-sorbitol in a flask scale experiment.

As shown in Table 11, overexpression of SDH2 significantly (c.a. 2-3 times) improved the 2KLG production from L-sorbose in a flask scale experiment. SDH2 overexpression with D-sorbitol as substrate resulted in the formation of 2-keto-D-gluconic acid (2KDG) as well as 2KLG (Table 10).

Having now fully described the present invention in some detail by way of illustration and example for purposes of clarity of understanding, it will be obvious to one of ordinary skill in the art that the same can be performed by modifying or changing the invention with a wide and equivalent range of conditions, formulations and other parameters thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. An isolated nucleic acid molecule comprising a polynucleotide sequence encoding a fragment at least about 10 amino acids in length of the amino acid sequence of SEQ ID NO:5.

2. The isolated nucleic acid molecule of claim 1, wherein the polynucleotide sequence encodes a polypeptide having the amino acid sequence of SEQ ID NO:5.

3. An isolated nucleic acid molecule comprising a polynucleotide sequence comprising a fragment at least about 30 nucleotides in length of SEQ ID NO:1.

4. The isolated nucleic acid molecule of claim 3, wherein the polynucleotide sequence is SEQ ID NO:1.

5. An isolated nucleic acid molecule comprising a polynucleotide sequence at least about 95% identical to SEQ ID NO:1.

6. A vector comprising a nucleic acid molecule which comprises a polynucleotide sequence selected from the group consisting of:
a polynucleotide sequence encoding a fragment at least about 10 amino acids in length of the amino acid sequence of SEQ ID NO:5;
a polynucleotide sequence encoding a polypeptide having the amino acid sequence of SEQ ID NO:5;
a polynucleotide sequence comprising a fragment at least about 30 nucleotides in length of SEQ ID NO:1;
the polynucleotide sequence of SEQ ID NO:1; and
a polynucleotide at least about 95% identical to SEQ ID NO: 1.

7. A process for producing the vector of claim 6 which comprises:
(a) inserting the nucleic acid molecule of any one of claims 1-5 into a vector; and
(b) selecting and propagating said vector in a host cell.

8. The process according to claim 7, wherein said insertion comprises electroporation.

9. A host cell containing the vector of claim 6.

10. An isolated nucleic acid molecule comprising a polynucleotide sequence encoding a fragment at least about 10 amino acids in length of the amino acid sequence of SEQ ID NO:6.

11. The isolated nucleic acid molecule of claim 10, wherein the polynucleotide sequence encodes a polypeptide having the amino acid sequence of SEQ ID NO:6.

12. An isolated nucleic acid molecule comprising a polynucleotide sequence comprising a fragment at least about 30 nucleotides in length of SEQ ID NO:2.

13. The isolated nucleic acid molecule of claim 12, wherein the polynucleotide sequence is SEQ ID NO:2.

14. An isolated nucleic acid molecule comprising a polynucleotide sequence at least about 95% identical to SEQ ID NO:2.

15. A vector comprising a nucleic acid molecule which comprises a polynucleotide sequence selected from the group consisting of:
a polynucleotide sequence encoding a fragment at least about 10 amino acids in length of the amino acid sequence of SEQ ID NO:6;
a polynucleotide sequence encoding a polypeptide having the amino acid sequence of SEQ ID NO:6;
a polynucleotide sequence comprising a fragment at least about 30 nucleotides in length of SEQ ID NO:2;
the polynucleotide sequence of SEQ ID NO:2; and
a polynucleotide sequence at least about 95% identical to SEQ ID NO:2.

16. A process for producing the vector of claim 15 which comprises:
(a) inserting the nucleic acid molecule of any one of claims 10-14 into a vector; and
(b) selecting and propagating said vector in a host cell.

17. The process according to claim 16, wherein said insertion comprises electroporation.

18. A host cell containing the vector of claim 15.

19. An isolated nucleic acid molecule comprising a polynucleotide sequence encoding a fragment at least about 10 amino acids in length of the amino acid sequence of SEQ ID NO:7.

20. The isolated nucleic acid molecule of claim 19, wherein the polynucleotide sequence encodes a polypeptide having the amino acid sequence of SEQ ID NO:7.

21. An isolated nucleic acid molecule comprising a polynucleotide sequence comprising a fragment at least about 30 nucleotides in length of SEQ ID NO:3.

22. The isolated nucleic acid molecule of claim 21, wherein the polynucleotide sequence is SEQ ID NO:3.

23. An isolated nucleic acid molecule comprising a polynucleotide sequence at least about 95% identical to SEQ ID NO:3.

24. A vector comprising a nucleic acid molecule which comprises a polynucleotide sequence selected from the group consisting of:
a polynucleotide sequence encoding a fragment at least about 10 amino acids in length of the amino acid sequence of SEQ ID NO:7;
a polynucleotide sequence encoding a polypeptide having the amino acid sequence of SEQ ID NO:7;
a polynucleotide sequence comprising a fragment at least about 30 nucleotides in length of SEQ ID NO:3;
the polynucleotide sequence of SEQ ID NO:3; and
a polynucleotide sequence at least about 95% identical to SEQ ID NO:3.

25. A process for producing the vector of claim 24 which comprises:
(a) inserting the nucleic acid molecule of any one of claims 19-23 into a vector; and
(b) selecting and propagating said vector in a host cell.

26. The process according to claim 25, wherein said insertion comprises electroporation.

27. A host cell containing the vector of claim 24.

28. An isolated nucleic acid molecule comprising a polynucleotide sequence encoding a fragment at least about 10 amino acids in length of the amino acid sequence of SEQ ID NO:8.

29. The isolated nucleic acid molecule of claim 28, wherein the polynucleotide sequence encodes a polypeptide having the amino acid sequence of SEQ ID NO:8.

30. An isolated nucleic acid molecule comprising a polynucleotide sequence comprising a fragment at least about 30 nucleotides in length of SEQ ID NO:4.

31. The isolated nucleic acid molecule of claim 30, wherein the polynucleotide sequence is SEQ ID NO:4.

32. An isolated nucleic acid molecule comprising a polynucleotide sequence at least about 95% identical to SEQ ID NO:4.

33. A vector comprising a nucleic acid molecule which comprises a polynucleotide sequence selected from the group consisting of:
a polynucleotide sequence encoding a fragment at least about 10 amino acids in length of the amino acid sequence of SEQ ID NO:8;
a polynucleotide sequence encoding a polypeptide having the amino acid sequence of SEQ ID NO:8;
a polynucleotide sequence comprising a fragment at least about 30 nucleotides in length of SEQ ID NO:4;
the polynucleotide sequence of SEQ ID NO:4; and
a polynucleotide sequence at least about 95% identical to SEQ ID NO:4.

34. A process for producing the vector of claim 33 which comprises:
(a) inserting the nucleic acid molecule of any one of claims 28-32 into a vector; and
(b) selecting and propagating said vector in a host cell.

35. The process according to claim 34, wherein said insertion comprises electroporation.

36. A host cell containing the vector of claim 33.

37. An isolated nucleic acid molecule comprising a polynucleotide sequence comprising a fragment at least about 20 nucleotides in length of the polynucleotide sequence of SEQ ID NO:9.

38. The isolated nucleic acid molecule of claim 37 wherein said polynucleotide sequence has the complete nucleotide sequence of the DNA clone contained in KCTC Deposit No. 0913BP.

39. The isolated nucleic acid molecule of claim 37 comprising the polynucleotide sequence of SEQ ID NO:9.

40. An isolated nucleic acid molecule comprising a polynucleotide sequence at least about 95% identical SEQ ID NO:9.

41. A vector comprising a nucleic acid molecule which comprises a polynucleotide sequence selected from the group consisting of:
a polynucleotide sequence comprising a fragment at least about 20 nucleotides in length of the polynucleotide of SEQ ID NO:9;
a polynucleotide sequence having the complete nucleotide sequence of the DNA clone contained in KCTC Deposit No. 0913BP.
the polynucleotide sequence of SEQ ID NO:9; and
a polynucleotide sequence at least about 95% identical to SEQ ID NO:9.

42. A process for producing the vector of claim 41 which comprises:
(a) inserting the nucleic acid molecule of any one of claims 37-40 into the vector; and
(b) selecting and propagating said vector in a host cell.

43. The process according to claim 42, wherein said insertion comprises electroporation.

44. A host cell comprising the vector of claim 41.

45. An isolated nucleic acid molecule comprising a polynucleotide sequence comprising a fragment at least about 20 nucleotides in length of the polynucleotide sequence of SEQ ID NO:10.

46. The nucleic acid molecule of claim 45 wherein said polynucleotide sequence has the complete nucleotide sequence of the DNA clone contained in KCTC Deposit No. 0914BP.

47. The isolated nucleic acid molecule of claim 45 comprising the polynucleotide sequence of SEQ ID NO:10.

48. An isolated nucleic acid molecule comprising a polynucleotide sequence at least about 95% identical SEQ ID NO:10.

49. A vector comprising a nucleic acid molecule which comprises a polynucleotide sequence selected from the group consisting of:
a polynucleotide sequence comprising a fragment at least about 20 nucleotides in length of the polynucleotide of SEQ ID NO:10;
a polynucleotide sequence having the complete nucleotide sequence of the DNA clone contained in KCTC Deposit No. 0914BP.
the polynucleotide sequence of SEQ ID NO:10; and
a polynucleotide sequence at least about 95% identical to SEQ ID NO:10.

50. A process for producing the vector of claim 49 which comprises:
(a) inserting the nucleic acid molecule of any one of claims 45-48 into the vector; and
(b) selecting and propagating said vector in a host cell.

51. The process according to claim 50, wherein said insertion comprises electroporation.

52. A host cell comprising the vector of claim 49.

53. An isolated nucleic acid molecule comprising a polynucleotide sequence comprising a fragment at least about 20 nucleotides in length of the polynucleotide of SEQ ID NO:11.

54. The nucleic acid molecule of claim 53 wherein said polynucleotide sequence has the complete nucleotide sequence of the DNA clone contained in KCTC Deposit No. 0915BP.

55. The isolated nucleic acid molecule of claim 53 comprising the polynucleotide sequence of SEQ ID NO:11.

56. An isolated nucleic acid molecule comprising a polynucleotide sequence at least about 95% identical SEQ ID NO:11.

57. A vector comprising a nucleic acid molecule which comprises a polynucleotide sequence selected from the group consisting of:
a polynucleotide sequence comprising a fragment at least about 20 nucleotides in length of the polynucleotide of SEQ ID NO:11;
a polynucleotide sequence having the complete nucleotide sequence of the DNA clone contained in KCTC Deposit No. 0915BP.
the polynucleotide seqeuence of SEQ ID NO:11; and
a polynucleotide sequence at least about 95% identical to SEQ ID NO:11.

58. A process for producing the vector of claim 57 which comprises:
(a) inserting the nucleic acid molecule of any one of claims 53-56 into the vector; and
(b) selecting and propagating said vector in a host cell.

59. The process according to claim 58, wherein said insertion comprises electroporation.

60. A host cell comprising the vector of claim 57.

61. A process for the production of L-sorbose from D-sorbitol comprising:
(a) transforming a host cell with at least one isolated nucleotide sequence selected from the group consisting of:
a polynucleotide sequence encoding the polypeptide sequence of SEQ ID NO:5;
a polynucleotide sequence encoding the polypeptide sequence of SEQ ID NO:6;
a polynucleotide sequence encoding the polypeptide sequence of SEQ ID NO:7; and
a polynucleotide sequence encoding the polypeptide sequence of SEQ ID NO:8;
(b) selecting and propagating said transformed host cell; and
(c) recovering L-sorbose.

62. The process according to claim 61, wherein the at least one isolated nucleotide sequence is selected from the group consisting of:
a polynucleotide sequence comprising the polynucleotide sequence of SEQ ID NO:1;
a polynucleotide sequence comprising the polynucleotide sequence of SEQ ID NO:2;
a polynucleotide sequence comprising the polynucleotide sequence of SEQ ID NO:3; and
a polynucleotide sequence comprising the polynucleotide sequence of SEQ ID NO:4.

63. The process of claim 61 or claim 62, wherein said host cell is Ketogulonigenium.

64. A process for increasing the production of 2-keto-L-gulonic acid comprising:
(a) transforming a host cell with at least one isolated nucleotide sequence selected from the group consisting of
a polynucleotide sequence encoding the polypeptide sequence of SEQ ID NO:5;
a polynucleotide sequence encoding the polypeptide sequence of SEQ ID NO:6;
a polynucleotide sequence encoding the polypeptide sequence of SEQ ID NO:7; and
a polynucleotide sequence encoding the polypeptide sequence of SEQ ID NO:8;
(b) selecting and propagating said transformed host cell; and
(c) recovering 2-keto-L-gulonic acid.

65. The process according to claim 64, wherein the at least one isolated nucleotide sequence is selected from the group consisting of:
a polynucleotide sequence comprising the polynucleotide sequence of SEQ ID NO:1;
a polynucleotide sequence comprising the polynucleotide sequence of SEQ ID NO:2;
a polynucleotide sequence comprising the polynucleotide sequence of SEQ ID NO:3; and
a polynucleotide sequence comprising the polynucleotide sequence of SEQ ID NO:4;

66. The process of claim 64 or claim 65, wherein said host cell is Ketogulonigenium.

67. An isolated polypeptide comprising a polypeptide sequence at least about 10 amino acids in length of the amino acid sequence of SEQ ID NO:5.

68. The isolated polypeptide of claim 67 encoded by the polynucleotide sequence of SEQ ID NO:1.

69. The isolated polypeptide of claim 67 encoded by the amino acid sequence of SEQ ID NO:5.

70. A process for producing a polypeptide comprising:
(a) growing the host cell of claim 9;
(b) expressing the polypeptide of any one of claims 67-69; and
(b) isolating said polypeptide.

71. An isolated polypeptide comprising a polypeptide sequence at least about 10 amino acids of the polypeptide sequence of SEQ ID NO:6.

72. The isolated polypeptide of claim 71 encoded by the polynucleotide sequence of SEQ ID NO:2.

73. The isolated polypeptide of claim 71 encoded by the amino acid sequence of SEQ ID NO:6.

74. A process for producing a polypeptide comprising:
(a) growing the host cell of claim 18;
(b) expressing the polypeptide of any one of claim 71-73; and
(c) isolating said polypeptide.

75. An isolated polypeptide comprising a polypeptide sequence at least about 10 amino acids of the polypeptide sequence of SEQ ID NO:7.

76. The isolated polypeptide of claim 75 encoded by the polynucleotide sequence of SEQ ID NO:3.

77. The isolated polypeptide of claim 75 encoded by the amino acid sequence of SEQ ID NO:7.

78. A process for producing a polypeptide comprising:
(a) growing the host cell of claim 27;
(b) expressing the polypeptide of any one of claim 75-77; and
(c) isolating said polypeptide.

79. An isolated polypeptide comprising a polypeptide sequence at least about 10 amino acids of the polypeptide sequence of SEQ ID NO:8.

80. The isolated polypeptide of claim 79 encoded by the polynucleotide sequence of SEQ ID NO:4.

81. The isolated polypeptide of claim 79 encoded by the amino acid sequence of SEQ ID NO:8.

82. A process for producing a polypeptide comprising:
(a) growing the host cell of claim 36;
(b) expressing the polypeptide of any one of claim 79-81; and
(c) isolating said polypeptide.
